(19) United States
(12) Patent Application Publication
(10) Pub. No.: US 2016/0108458 A1
(43) Pub. Date: Apr. 21, 2016

(54) MULTIPLEXED DETECTION AND QUANTIFICATION OF NUCLEIC ACIDS IN SINGLE-CELLS

(71) Applicant: The Board of Trustees of the Leland Stanford Junior University, Stanford, CA (US)

(72) Inventors: Andreas Philipp Frei, San Francisco, CA (US); Garry P. Nolan, Redwood City, CA (US); Pier Federico Gherardini, Palo Alto, CA (US); Felice Alessio Bava, Menlo Park, CA (US)

(21) Appl. No.: 14/875,454

(22) Filed: Oct. 5, 2015

Related U.S. Application Data
(60) Provisional application No. 62/060,210, filed on Oct. 6, 2014.

(51) Int. Cl. C12Q 1/68

(52) U.S. Cl. CPC C12Q 1/682

(57) ABSTRACT

Proximity Ligation Assay for RNA (PLAYR) provides cost-efficient detection of specific nucleic acids in single cells, and may be combined with flow cytometry to simultaneously analyze large numbers of cells for a plurality of nucleic acids, e.g. at least one, to up to 5, up to 10, up to 15, up to 20 or more transcripts can be simultaneously analyzed, at a rate of up to about 50, 100, 250, 500 or more cells/second. An advantage of PLAYR includes the ability to simultaneously analyze multiple nucleic acids and proteins in single cells, as the method is compatible with conventional antibody staining for proteins, intracellular phosphorylation sites, and other cellular antigens. This enables the simultaneous detection of multiple nucleic acid molecules in combination with additional cellular parameters.

### GOVERNMENT RIGHTS

This invention was made with Government support under contracts A1100627 and HHSN268201000034C awarded by the National Institutes of Health. The Government has certain rights in the invention.

### BACKGROUND OF THE INVENTION

High-throughput measurements of gene expression using microarray technology or high throughput sequencing contribute tremendously to our understanding of how genetic networks coordinately function in normal cells and tissues and how they malfunction in disease. Such measurements allow one to infer the function of genes based on their expression patterns, to detect which genes have altered expression in disease, and to identify expression signatures that are predictive of disease progression. However, bulk transcriptome measurements only inform on the average gene expression in a sample. Thus, in a complex sample containing several cell types with different gene expression signatures, only the most abundant signature but not necessarily the most meaningful will be captured. Accordingly, the variability in single-cell gene expression in most biological systems and especially in tissues and tumors generates a need for techniques aimed at characterizing gene expression programs in individual cells of interest.

The increasing appreciation for the importance of single-cell measurements is reflected in the vast number of single-cell analysis platforms that have been successfully commercialized in recent years, including mass cytometry and microfluidic-based approaches. While flow cytometry provides an excellent platform for the detection of proteins in single cells using antibodies, no comparable solution exists for the detection of nucleic acids. Microfluidic technologies for the detection and quantification of mRNA in single cells are very costly and their throughput is several orders of magnitude lower compared with what can be achieved for proteins using flow cytometry.

To overcome the limitations of bulk analyses, a number of technologies have been developed that measure gene expression in single cells. In one such method, up to 20 short oligonucleotide probe pairs hybridize in adjacent positions to a target RNA. These binding events are subsequently amplified using branched DNA technology, where the addition of sets of oligonucleotides in subsequent hybridization steps gives rise to a branched DNA molecule. The presence of such a branched DNA structure can then be detected and quantified by flow cytometry using a fluorescent probe. This technology enables the detection of only few RNA copy numbers in millions of single cells but is currently limited to the simultaneous detection of small numbers of measured transcripts. Furthermore, the protocol is long and laborious and the buffers used are not compatible with some fluorophores commonly used in flow cytometry and cannot be used at all in mass cytometry.

Another method (Larsson et al. (2010) Nature Methods), uses padlock probes, i.e. linear probes that can be converted into a circular DNA molecule by target-dependent ligation upon hybridization to a target RNA molecule. The resulting circularized single-stranded DNA probe can then be amplified using the enzyme phi29 polymerase in a process termed Rolling Circle Amplification (RCA). This process produces a single-stranded DNA molecule containing hundreds of complementary tandem repeats of the original DNA circle. This RCA product can be made visible through the addition of fluorescently labeled detection probes that will hybridize to a detection sequence in the product. This technology enables the multiplex detection of transcripts but requires reverse transcription of target mRNAs using specific primers and RNAseH digestion of the original transcript before hybridization of the padlock probe. Therefore, it introduces additional variability in the assay and requires the design and optimization of both probes and primers.

Another commercially available solution for single-cell mRNA measurements is based on the physical separation of single cells using a microfluidic device followed by library preparation and sequencing. This is currently the only genome-wide solution but the very limited throughput (96 cells per run) makes it unsuitable for the analysis of samples with multiple cell populations such as blood samples or tumors. Additionally, the technology is expensive compared to the other approaches, and does not allow for the simultaneous detection of proteins and mRNAs in the same cell.

There is a need for methods that can provide information on multiple transcripts in single cells, particularly that can be usefully combined with protein analysis. Such methods can help analyze how biological networks coordinately function in normal and diseased cells and tissues. The present invention addresses this need.

### PUBLICATIONS

Larsson et al. In situ detection and genotyping of individual mRNA molecules. Nat. Methods 7, 395-397 (2010). Player et al. Single-copy gene detection using branched DNA (bDNA) in situ hybridization. J. Histochem. Cytochem. 49, 603-612 (2001). Porichis, F. et al. High-throughput detection of miRNAs and gene-specific mRNA at the single-cell level by flow cytometry. Nature Communications 5, 5641 (2014). Bendall, S. C. et al. Single-cell mass cytometry of differential immune and drug responses across a human hematopoietic continuum. Science 332, 687-696 (2011). Wolf-Yadlin, A. et al. Effects of HER2 overexpression on cell signaling networks governing proliferation and migration. Mol Syst Biol 2, 54 (2006). Angelo, M. et al. Multiplexed ion beam imaging of human breast tumors. Nat Med 20, 436-442 (2014). Fredriksson, S. et al. Protein detection using proximity-dependent DNA ligation assays. Nat Biotechnol 20, 473-477 (2002). Soderberg, O. et al. Direct observation of individual endogenous protein complexes in situ by proximity ligation. Nat. Methods 3, 995-1000 (2006).

International patent applications WO2012/160083; WO2001/061037; WO2013/173774.

### SUMMARY OF THE INVENTION

Methods and compositions are provided for multiplexed analysis of target nucleic acids in single cells by a method herein termed PLAYR (Proximity Ligation Assay for RNA). The methods of the invention enable cost-efficient detection of specific nucleic acids in single cells, and may be combined with flow cytometry or mass cytometry to simultaneously analyze large numbers of cells for a plurality of nucleic acids, e.g. at least one, to up to 5, up to 10, up to 15, up to 20, up to 30, up to 40 or more transcripts can be simultaneously analyzed, at a rate of up to about 50, 100, 250, 500, up to 750, up to 1000 or more cells/second. An advantage of PLAYR includes the ability to simultaneously analyze multiple nucleic acids and proteins in single cells, as the method is compatible with conventional antibody staining for proteins, intracellular phosphorylation sites, and other cellular antigens. This enables the simultaneous detection of multiple nucleic acid molecules in combination with additional cellular parameters. It can be combined with various different platforms, including without limitation FACS, mass cytometry, microscopy, nano-SIMS imaging, and the like.

In the methods of the invention, a pair of short oligonucleotide probes are designed that specifically hybridize to adjacent regions of a target nucleic acid. Target nucleic acids include, without limitation, mRNA, pre-mRNA, rRNA, miRNA, lincRNA, denatured DNA, and the like. Each probe in the pair further comprises a linker and a “PLAYR 1” or “PLAYR 2” sequence that does not hybridize to the target nucleic acid. When the probes are bound to the target nucleic acid, the PLAYR 1 and PLAYR 2 regions of the probe act as template for the hybridization, circularization, and ligation of the components of a DNA padlock probe that are added in a subsequent step. The resulting circular single-stranded DNA product is amplified by rolling circle amplification (RCA), which produces a single-stranded DNA molecule containing complementary tandem repeats of the original DNA circle. The amplification product is detected with a complementary detection probe labeled with a detectable marker, e.g. fluorophore, metal conjugate, etc. A high level of specificity results from the requirement that both probes hybridize to adjacent locations for the amplification reaction to take place, resulting in excellent specificity, low background, and high signal-to-noise ratios.

In some embodiments, a method is provided for determining the abundance of a target nucleic acid in a single cell, the method comprising contacting a fixed and permeabilized cell with at least one pair of oligonucleotide primers under conditions permissive for specific hybridization, wherein each oligonucleotide in the pair comprises: a target binding region that hybridizes to the target nucleic acid; a spacer region that does not bind to the target nucleic acid or to any region of a padlock probe; and an PLAYR 1 or PLAYR 2 region that specifically binds to the padlock probe, wherein each padlock probe comprises two polynucleotides: a backbone and an insert, and wherein the PLAYR 1 or PLAYR 2 region binds to both insert and backbone; washing the cells free of unbound primers; contacting the cells with backbone and insert polynucleotides under conditions permissive for specific hybridization; washing the cells free of unbound backbone insert; performing a ligation reaction, in which bound backbone insert polynucleotides are ligated to generate a circle; amplifying the ligated backbone/insert circle by rolling circle amplification; washing the cells free of polymerase; hybridizing detection primers to the amplified circle; washing the cells free of unbound detection probes, and quantitating the level of bound detection primers to determine the abundance of the target nucleic acid. Quantitation may include use of a detection probe conjugated to a fluorescent or metal label, and determination of the level of fluorescent or metal label present, e.g. by nano-SIMS, mass cytometry, FACS, etc. In many embodiments, a plurality of target nucleic acids are simultaneously analyzed.

In some embodiments of the invention, PLAYR is used in combination with cytometry gating on specific cell populations, as defined by other cellular parameters measured simultaneously, for example in combination with antibody staining and mass cytometry or FACS to define a subpopulation of interest. In such embodiments, a complex cell population may be analyzed, e.g. a biopsy or blood sample potentially including immune cells, progenitor or stem cells, cancer cells, etc. For example, a method is provided for determining the abundance of one or more target nucleic acids in a defined cell type within a complex cell population, where the quantification of detection probes is combined with detection of cellular markers, including without limitation protein markers, that serve to define the cell type of interest.

In other embodiments, the methods of the invention are used for multiplexed detection and quantification of specific splice variants of mRNA transcripts in single cells.

In yet another embodiment, the methods of the invention are combined with Proximity Ligation Assay (PLA) for the simultaneous detection and quantification of nucleic acid molecules and protein-protein interactions.

With prior denaturation of endogenous cellular DNA (by heat, enzymatic methods, or any other suitable procedure), the technology is modified for the detection of specific DNA sequences (genotyping of single cells). In this adaptation, the technology enables the quantification of gene copy number variations as well as the detection of genomic translocation/fusion events. For example, in the detection of a fusion event, if a first gene is fused to a second gene the PLAYR method can be adapted, where one or more primers are targeted to gene 1, with an PLAYR 1 sequence; and one or more primers are targeted to gene 2 with an PLAYR 2 sequence. A signal is obtained only when the fusion transcript is present, as the individual probes do not give rise to an amplification product. A plurality of individual primers may be designed for each of gene 1 and gene 2, e.g. 2, 3, 4, 5, 6 or more.

### BRIEF DESCRIPTION OF THE DRAWINGS

FIG. 1: Overview of the PLAYR technology, see text for details.

FIG. 2: Varying the Insert and PLAYR1/PLAYR2 sequence allows probes targeting different transcripts to be barcoded. This enables the multiplexed detection of multiple transcripts in the same cell.

FIG. 3A-3B: FIG. 3A) PLAYR specifically detects target transcripts. Jurkat cells express CD3E and do not express CD10 and CD179a. Conversely Nalm-6 cells express CD10 and CD179a but not CD3E. The histograms depict the fluorescence intensity of the two cell lines when treated with probes specifically targeting these transcripts. A strong positive signal is only observed in the cell line expressing the transcript targeted by the PLAYR probes. Cells were also incubated with two single probes targeting the Actin and Gapdh transcript respectively. These two probes never hybridize in close proximity, as they target different transcripts. Accordingly no signal is observed. FIG. 3B) The signal can be increased by using multiple probe pairs targeting the same transcript.

FIG. 4: The PLAYR signal decreases with the distance between the two probes in a pair. Multiple adjacent probe pairs spanning a transcript were designed. Each PLAYR1 probe was then tested in combination with all the PLAYR2 probes from all the other pairs. The y-axis represents the ratio between the signal obtained with a given PLAYR1/PLAYR2 combination, and the signal obtained with the corresponding adjacent PLAYR1/PLAYR2 pair (i.e. the one that was originally designed as an adjacent pair). There is a clear tendency for the signal to decrease as the distance between the PLAYR1 and PLAYR2 probes increases.

FIG. 5: Simultaneous detection of nine transcripts in Jurkat cells. Nine different inserts are used to barcode probe sets targeting three different transcripts (CD90, CD3, KRAS, NRAS, PLCG, LCK, ZAP70, ACTB, GAPDH). Nine different detection oligonucleotides, specific for each Insert system, were also conjugated to a polymer chelating nine different stable transition element isotopes (150Nd, 162Dy, 153Eu, 156Gd, 148Nd, 176Yb, 160Gd, 167Er, 168Er respectively). The probe sets and detection oligonucleotides for each gene were incubated simultaneously and the signal intensity was measured on a CyTOF mass cytometer.

FIG. 6A-6D: PLAYR enables the simultaneous quantification of specific transcripts and proteins in single cells FIG. 6A) Main steps of the PLAYR protocol: 1) Fixation of cells captures their native state and permeabilization enables intracellular antibody staining and blocking of endogenous RNAses with inhibitors. 2) PLAYR probe pairs are added for proximal hybridization to target transcripts. 3) Backbone and insert oligonucleotides are added and form a circle if hybridized to PLAYR probes that are in close proximity (bound to a transcript). Insert sequences serve as cognate barcodes for targeted transcripts. 4) Backbone and insert oligonucleotides are ligated into a single-stranded DNA circle by T4 DNA ligase. 5) Rolling circle amplification of the DNA circle by phy29 polymerase. 6) Detection of rolling circle amplicons with suitably labeled oligonucleotides that bind to the insert regions. FIG. 6B) Detection of transcripts for three housekeeping genes that span a wide abundance range in U937 cells by mass cytometry. FIG. 6C) Quantification of CCL4 and IFNG mRNA by PLAYR and qPCR in NKL cells after stimulation with PMA/ionomycin. FIG. 6D) Simultaneous IFNG mRNA and protein quantification by mass cytometry in NKL cells at indicated time points after stimulation with PMA/ionomycin.

FIG. 7A-7C: Highly multiplexed measurement of different transcripts in single cells (FIG. 7A) Detection of 14 different transcripts in Jurkat cells by mass cytometry. PLAYR probes to transcripts not expressed in T cells (HLA-DRA) or to those encoding T cell surface markers, T cell signaling molecules, and housekeeping proteins of different abundance levels were used. Each row represents a sample to which probe pairs for one gene only or all genes simultaneously (bottom row) were added. Each column represents a mass cytometry acquisition channel that monitors a metal reporter used to detect transcripts of a given gene. Non-cognate probes that are using the same insert system but bind to different target transcripts were included as an additional control (CTL). b) NKL cells were primed with IL2/IL12/IL18 and stimulated with PMA/ionomycin for 3 hours. Contour plots display co-expression of NKL effector transcripts as measured by mass cytometry. FIG. 7C) 10000 cells were randomly sampled from the data in (FIG. 7B) and transcript expression was represented in heat map format. Each column corresponds to a single cell and rows denote different effector transcripts. Rows and columns of the heat map were clustered for visual clarity.

FIG. 8A-8E: Highly multiplexed measurement of transcripts within cell types defined by other transcripts or protein epitopes. (FIG. 8A) viSNE analysis of embryonic stem cells, differentiating embryonic stem cells, and embryonic fibroblasts of mice based on expression of 15 transcripts (CD44, MKI67, CDH1, CD47, KLF4, ESRRB, ACTB, SOX2, LINCENC1, ZFP42, SALL4, CD9, POU5F1 (OCT4), THY1, NANOG) with overlays showing the location of the three cell populations. FIG. 8B) Color-coded expression levels of selected transcripts used to construct the viSNE map. FIG. 8C) viSNE analysis of PBMCs based on expression of 10 surface protein markers (CD19, CD4, CD8, CD20, PTPRC (CD45), PTPRCRA (CD45RA), CD33, ITGAX (CD11c), CD3, HLA-DRA) with overlays showing the location of major cell populations. FIG. 8D) Expression of selected proteins and the corresponding transcripts was overlaid in the viSNE map shown in (FIG. 8C) and color-coded by signal intensity. FIG. 8E) Contour plots displaying correlations of protein and transcript levels for HLA-DRA and ITGAX in individual PBMCs.

FIG. 9A-9E: Measurements of cytokine transcript induction in human PBMCs. (FIG. 9A) Mass cytometry gating strategy for human PBMCs. FIG. 9B) Heat map representing the mean expression values of cytokine transcripts at different time points after stimulation with LPS in different cellular populations defined by protein surface markers. FIG. 9C) Cytokine expression in the CD14+ monocyte population as measured by fluorescence flow cytometry. FIG. 9D) Cytokine transcript expression in the CD14+ monocyte population as measured by mass cytometry. FIG. 9E) Contour plots showing interleukin 8 (CXCL8) and tumor necrosis factor alpha (TNF) transcript expression in CD14+ monocytes.

FIG. 10A-10C: Single-cell resolution map of cytokine induction in human PBMCs. PBMCs were stimulated with LPS and analyzed after 4 hours. Cells were analyzed with antibodies against cell surface proteins (CD19, CD38, CD4, CD8, CD7, CD14, IL3RA (CD123), PTPRC (CD45), PTPRCRA (CD45RA), CD33, ITGAX (CD11c), FCGR3A (CD16), CD3, CD20, HLA-DRA, NCAM1 (CD56) and phosphorylation sites pP38 MAPK (pT180/pY182), pERK1/2 (pT202/pY204). FIG. 10A) viSNE map based on cell surface marker expression with overlays showing the location of major cell populations. FIG. 10B) Selected protein markers used to define myeloid cell populations and MAPK signaling were color-coded by expression level. FIG. 10C) Measurements for 8 different cytokine transcripts were overlaid and color-coded by expression level.

FIG. 11: Graphical display of the PLAYRDesign software tool for user-friendly design of PLAYR probe pairs. Each potential probe is represented by a red rectangle. The Primer3 score of each probe is represented by a color gradient from light pink to red, where red probes have higher scores and are preferred over light red probes. The position of probes along the transcript is represented together with sequence features that can guide probe selection. Different graphs represent: maximum sequence identity of BLAST matches to a database of repetitive sequences (red); maximum sequence identity of BLAST matches to other transcripts (blue); predicted melting temperature in a window of 20 residues (green); number of ESTs that skip an exon but include the exons flanking it (blue). The actual melting temperature of probes is independently calculated by Primer3, while the purpose of the green graph is to give an indication on whether certain regions of the transcript have a melting temperature that is too low or too high to be amenable for probe design. Blue and red graphs represent sequence features that are not considered in the scoring of Primer3 probes.

FIG. 12: Specificity control experiments for PLAYR. Detection of the Beta-actin transcript in Jurkat cells (ACTB). No signal is detected when PLAYR is performed in absence of probes (NO PROBES), in absence of insert (NO INSERT), in absence of backbone (NO BACKBONE), in absence of ligase (NO LIGATION), in absence of detection oligo (NO DETECTION OLIGO), in presence of probes directed against the anti-sense Beta-actin transcript (SENSE PROBES), in presence of probes with the same half of the insert-complementary sequence (ORIENTATION CONTROL), or in presence of non-cognate probe pairs targeting different transcripts (ACTB and GAPDH, GENE-SPECIFICITY CONTROL). Signals were detected by flow cytometry. 4 probe pairs were used per gene.

FIG. 13A-13B: Detection of specific transcripts in single cells by flow cytometry using multiple probe pairs. FIG. 13A) Detection of CD10 and CD3E by PLAYR. Jurkat and NALM-6 cells were incubated with the indicated number of probe pairs and analyzed by flow cytometry. FIG. 13B) The intensity of PLAYR signals depends on the distance between PLAYR probe binding sites on a target transcript. Multiple adjacent probe pairs spanning a transcript were designed and tested in all possible pairwise combinations. The x-axis represents the distance between each pair of probes, and the y-axis represents the ratio between the signal obtained with a given combination and the signal obtained with the corresponding adjacent probe (i.e., the one that was originally designed to be used in the pair).

FIG. 14: Fluorescence flow cytometry gating strategy for human PBMCs.

### DETAILED DESCRIPTION OF THE EMBODIMENTS

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

Target Nucleic Acid.

As used herein, a target nucleic acid is any polynucleotide nucleic acid molecule (e.g., DNA molecule; RNA molecule, modified nucleic acid, etc.) present in a single cell. In some embodiments, the target nucleic acid is a coding RNA (e.g., mRNA). In some embodiments, the target nucleic acid is a non-coding RNA (e.g., tRNA, rRNA, microRNA (miRNA), mature miRNA, immature miRNA; etc). In some embodiments, the target nucleic acid is a splice variant of an RNA molecule (e.g., mRNA, pre-mRNA, etc.) in the context of a cell. A suitable target nucleic acid can therefore be an unspliced RNA (e.g., pre-mRNA, mRNA), a partially spliced RNA, or a fully spliced RNA, etc.

Target nucleic acids of interest may be variably expressed, i.e. have a differing abundance, within a cell population, wherein the methods of the invention allow profiling and comparison of the expression levels of nucleic acids, including without limitation RNA transcripts, in individual cells.

A target nucleic acid can also be a DNA molecule, e.g. a denatured genomic, viral, plasmid, etc. For example the methods can be used to detect copy number variants, e.g. in a cancer cell population in which a target nucleic acid is present at different abundance in the genome of cells in the population; a virus-infected cells to determine the virus load and kinetics, and the like.

Target Specific Oligonucleotide Primer Pairs.

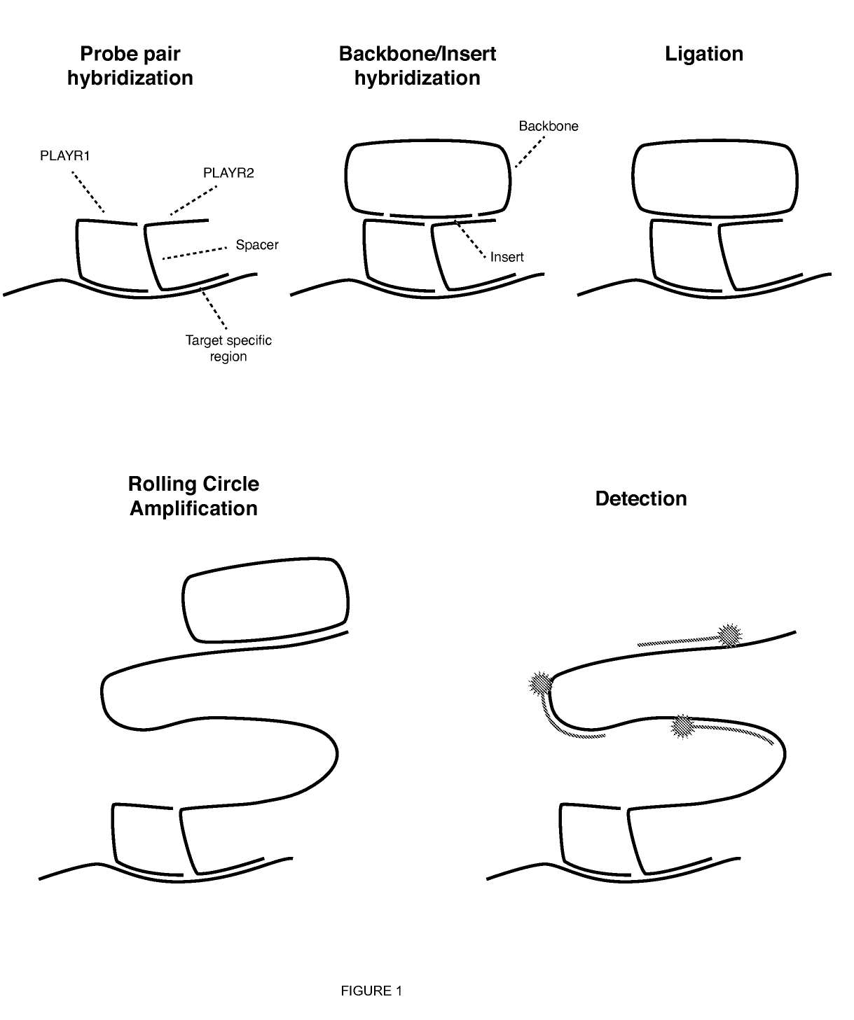

In the methods of the invention, one or more pairs of target specific oligonucleotide primers are contacted with a cell comprising target nucleic acids. Each oligonucleotide in a pair comprises 3 regions: a target binding site, a spacer, and a padlock probe binding site, which is referred to herein as PLAYR 1 or PLAYR 2. See FIG. 1. A plurality of oligonucleotide pairs can be used in a reaction, where one or more pairs specifically bind to each target nucleic acid. For example, two primer pairs can be used for one target nucleic acid in order to improve sensitivity and reduce variability. It is also of interest to detect a plurality of different target nucleic acids in a cell, e.g. detecting up to 2, up to 3, up to 4, up to 5, up to 6, up to 7, up to 8, up to 9, up to 10, up to 12, up to 15, up to 18, up to 20, up to 25, up to 30, up to 40 or more distinct target nucleic acids. The primers are typically denatured prior to use, typically by heating to a temperature of at least about 50° C., at least about 60° C., at least about 70° C., at least about 80° C., and up to about 99° C., up to about 95° C., up to about 90° C.

The target binding site binds to a region of the target nucleic acid. In a pair, each target site is different, and the pair are complementary adjacent sites on the target nucleic acid, e.g. usually not more than 10 nt distant, not more than 9, 8, 7, 6, 5, 4, 3, 2, or 1 nt. distant from the other site, and may be contiguous sites. Target sites are typically present on the same strand of the target nucleic acid in the same orientation. Target sites are also selected to provide a unique binding site, relative to other nucleic acids present in the cell. Each target site is generally from about 18 to about 25 nt in length, e.g. from about 18 to 23, from about 18-21, etc. The pair of oligonucleotide probes are selected such that each probe in the pair has a similar melting temperature for binding to its cognate target site, e.g. the Tm may be from about 50° C., from about 52° C., from about 55° C., and up to about 70° C., up to about 72° C., up to about 70° C., up to about 65° C., up to about 62° C., and may be from about 58° to about 62° C. The GC content of the target site is generally selected to be no more than about 20%, no more than about 30%, no more than about 40%, no more than about 50%, no more than about 60%, no more than about 70%,

The spacer region is between the target specific region and the PLAYR 1 or PLAYR 2 region, and is preferably not complementary to target nucleic acids or the padlock probe, and is selected to provide for a low background. In some embodiments the spacer is a poly-A tract. The spacers are typically of even length on both probes in the pair, and may be from about 2 to about 20 nt in length, e.g. up to about 20, up to about 18, up to about 15, up to about 12, up to about 10, up to about 7, up to about 5, up to about 3 nt. in length. In some embodiments the spacer is from 8- to 12 nt in length.

The PLAYR 1 or PLAYR 2 regions specifically bind to components of the padlock probes, and are selected to distribute the binding between the insert and backbone sequences. The sequence of the PLAYR region is arbitrary, and can be chosen to provide bar-coding information, etc. Different PLAYR regions used in a reaction, particularly a multiplex reaction, may be selected to provide equivalent melting temperatures, e.g. Tm that are not more than 1-2 degrees different. The distribution in sequence complementary to insert and complementary to backbone is roughly equal, for example where 9-13 nt. are complementary to each of the insert and backbone of the padlock probe, and where the backbone and insert of the padlock probe hybridize to contiguous sequences on the PLAYR site. It is preferable for the PLAYR 1 sequence to differ from the PLAYR 2 sequence.

Padlock Probe.

As shown in FIG. 1, the two polynucleotides of the padlock probe are complementary to the PLAYR 1 and PLAYR 2 regions, where the PLAYR 1 or PLAYR 2 sequence is complementary to adjacent sequences of the insert and backbone, and where the PLAYR 1 binding sequence of the insert is adjacent to the PLAYR 2 binding sequence of the insert. When both PLAYR 1 and PLAYR 2 probes are present and properly aligned, the insert and backbone form an open circular molecule that can be ligated to create a closed circle. The insert sequence is therefore fully complementary to the insert binding sequences of the PLAYR 1 and RL2 probe regions, and is generally from about 18 to about 25 nt in length, e.g. from about 18 to 23, from about 18-21, etc.

Where a plurality of target nucleic acids are being detected, each insert sequence is specific for each target specific primer pair. In other words, all inserts are substantially different from the other in sequence, generally having not more than 4 nt in a common string. This ensures that the resulting amplification products barcode for the detected target and can be detected with different detection oligonucleotides conjugated to corresponding reporters.

The backbone of the padlock probe is selected to be of a length that allows circularization with steric strain, with low background hybridization to sequences present in the cell of interest, with the exception of the specific PLAYR 1/2 binding sites. The terminal ends of the backbone specifically bind to a portion of the PLAYR 1 and PLAYR 2 sequences, e.g. a region of about 6-12 nt in length. The overall length of the backbone is from about 50 to about 250 nt. in length, e.g. from about 50 to about 200, from about 50 to about 150, from about 50 to about 100 nt. in length.

Ligase. The term “ligase” as used herein refers to an enzyme that is commonly used to join polynucleotides together or to join the ends of a single polynucleotide. Ligases include ATP-dependent double-strand polynucleotide ligases, NAD+-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases include bacterial ligases such as E. coli DNA ligase and Taq DNA ligase, Ampligase® thermostable DNA ligase (Epicentre®Technologies Corp., part of Illumina®, Madison, Wis.) and phage ligases such as T3 DNA ligase, T4 DNA ligase and T7 DNA ligase and mutants thereof.

Rolling Circle Amplification.

A single-stranded, circular polynucleotide template is formed by ligation of the backbone and insert polynucleotides, which circular polynucleotide comprises a region that is complementary to the PLAYR 1 and PLAYR 2 sequences. Upon addition of a DNA polymerase in the presence of appropriate dNTP precursors and other cofactors, either the PLAYR 1 or the PLAYR 2 sequence, which can both act as primers for the polymerase, is elongated by replication of multiple copies of the template. This amplification product can be readily detected by binding to a detection probe.

Techniques for rolling circle amplification are known in the art (see, e.g., Baner et al, Nucleic Acids Research, 26:5073-5078, 1998; Lizardi et al, Nature Genetics 19:226, 1998; Schweitzer et al. Proc. Natl Acad. Sci. USA 97:10113-119, 2000; Faruqi et al, BMC Genomics 2:4, 2000; Nallur et al, Nucl. Acids Res. 29:el 18, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801). In some embodiments the polymerase is phi29 DNA polymerase.

Detection Probe.

The presence and quantitation of an amplified PLAYR padlock sequence in a cell is determined by contacting the cell with an oligonucleotide probe under conditions in which the probe binds to the amplified product. The probe comprises a detectable label, that can be measured and quantitated. A labeled nucleic acid probe is a nucleic acid that is labeled with any label moiety. In some embodiments, the nucleic acid detection agent is a single labeled molecule (i.e., a labeled nucleic acid probe) that specifically binds to the amplification product. In some embodiments, the nucleic acid detection agent includes multiple molecules, one of which specifically binds to the amplification product. In such embodiments, when a labeled nucleic acid probe is present, the labeled nucleic acid probe does not specifically bind to the target nucleic acid, but instead specifically binds to one of the other molecules of the nucleic acid detection agent. A hybridization probe can be any convenient length that provides for specific binding, e.g. it may be from about 16 to about 50 nt. in length, and more usually is from about 18 nt. to about 30 nt. length.

A “label” or “label moiety” for a nucleic acid probe is any moiety that provides for signal detection and may vary widely depending on the particular nature of the assay. Label moieties of interest include both directly and indirectly detectable labels. Suitable labels for use in the methods described herein include any moiety that is indirectly or directly detectable by spectroscopic, photochemical, biochemical, immunochemical, electrical, optical, chemical, or other means. For example, suitable labels include antigenic labels (e.g., digoxigenin (DIG), fluorescein, dinitrophenol(DNP), etc.), biotin for staining with labeled streptavidin conjugate, a fluorescent dye (e.g., fluorescein, Texas red, rhodamine, a fluorophore label such as an ALEXA FLUOR® label, and the like), a radiolabel (e.g., ³H, ¹²⁵I, ³⁵S, ¹⁴C, or ³²P), an enzyme (e.g., peroxidase, alkaline phosphatase, galactosidase, and others commonly used in an ELISA), a fluorescent protein (e.g., green fluorescent protein, red fluorescent protein, yellow fluorescent protein, and the like), a synthetic polymer chelating a metal, a colorimetric label, and the like. An antigenic label can be incorporated into the nucleic acid on any nucleotide (e.g., A,U,G,C).

Fluorescent labels can be detected using a photodetector (e.g., in a flow cytometer) to detect emitted light. Enzymatic labels are typically detected by providing the enzyme with a substrate and detecting the reaction product produced by the action of the enzyme on the substrate, colorimetric labels can be detected by simply visualizing the colored label, and antigenic labels can be detected by providing an antibody (or a binding fragment thereof) that specifically binds to the antigenic label. An antibody that specifically binds to an antigenic label can be directly or indirectly detectable. For example, the antibody can be conjugated to a label moiety (e.g., a fluorophore) that provides the signal (e.g., fluorescence); the antibody can be conjugated to an enzyme (e.g., peroxidase, alkaline phosphatase, etc.) that produces a detectable product (e.g., fluorescent product) when provided with an appropriate substrate (e.g., fluorescent-tyramide, FastRed, etc.); etc.

Metal labels (e.g., Sm¹⁵², Tb¹⁵⁹, Er¹⁷⁰, Nd¹⁴⁶, Nd¹⁴², and the like) can be detected (e.g., the amount of label can be measured) using any convenient method, including, for example, nano-SIMS, by mass cytometry (see, for example: U.S. Pat. No. 7,479,630; Wang et al. (2012) Cytometry A. 2012 July; 81(7):567-75; Bandura et. al., Anal Chem. 2009 Aug. 15; 81(16):6813-22; and Ornatsky et. al., J Immunol Methods. 2010 Sep. 30; 361(1-2):1-20. As described above, mass cytometry is a real-time quantitative analytical technique whereby cells or particles are individually introduced into a mass spectrometer (e.g., Inductively Coupled Plasma Mass Spectrometer (ICP-MS)), and a resultant ion cloud (or multiple resultant ion clouds) produced by a single cell is analyzed (e.g., multiple times) by mass spectrometry (e.g., time of-flight mass spectrometry). Mass cytometry can use elements (e.g., a metal) or stable isotopes, attached as label moieties to a detection reagent (e.g., an antibody and/or a nucleic acid detection agent).

Nucleic Acids, Analogs and Mimetics.

In defining the component oligonucleotide primers, probes, etc., used in the methods of the invention, it is to be understood that such probes, primers etc. encompass native and synthetic or modified polynucleotides, particularly the probes, primers etc. that are not themselves substrates for enzymatic modification during the performance of the method, e.g. the target specific oligonucleotide primers, and the detection probes.

A modified nucleic acid has one or more modifications, e.g., a base modification, a backbone modification, etc, to provide the nucleic acid with a new or enhanced feature (e.g., improved stability). A nucleoside can be a base-sugar combination, the base portion of which is a heterocyclic base. Heterocyclic bases include the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In some cases, the respective ends of this linear polymeric compound can be further joined to form a circular compound. In addition, linear compounds may have internal nucleotide base complementarity and may therefore fold in a manner as to produce a fully or partially double-stranded compound. Within oligonucleotides, the phosphate groups can be referred to as forming the internucleoside backbone of the oligonucleotide. The linkage or backbone of RNA and DNA can be a 3′ to 5′ phosphodiester linkage.

Examples of suitable nucleic acids containing modifications include nucleic acids with modified backbones or non-natural internucleoside linkages. Nucleic acids having modified backbones include those that retain a phosphorus atom in the backbone and those that do not have a phosphorus atom in the backbone. Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity include a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some embodiments, a subject nucleic acid has one or more phosphorothioate and/or heteroatom internucleoside linkages, in particular —CH₂—NH—O—CH₂—, —CH₂—N(CH₃)—O—CH₂-(known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677. Suitable amide internucleoside linkages are disclosed in U.S. Pat. No. 5,602,240.

Also suitable are nucleic acids having morpholino backbone structures as described in, e.g., U.S. Pat. No. 5,034,506. For example, in some embodiments, a subject nucleic acid includes a 6-membered morpholino ring in place of a ribose ring. In some of these embodiments, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Also included are nucleic acid mimetics. The term “mimetic” as it is applied to polynucleotides encompasses polynucleotides where only the furanose ring or both the furanose ring and the internucleotide linkage are replaced with non-furanose groups, replacement of only the furanose ring is also referred to as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262.

Another class of suitable polynucleotide mimetic is based on linked morpholino units (morpholino nucleic acid) having heterocyclic bases attached to the morpholino ring. A number of linking groups have been reported that can link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41(14), 4503-4510). Morpholino-based polynucleotides are disclosed in U.S. Pat. No. 5,034,506. A variety of compounds within the morpholino class of polynucleotides have been prepared, having a variety of different linking groups joining the monomeric subunits.

Another suitable class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). The incorporation of CeNA monomers into a DNA chain increases the stability of a DNA/RNA hybrid. CeNA oligoadenylates formed complexes with RNA and DNA complements with similar stability to the native complexes. The incorporation CeNA structures into natural nucleic acid structures was shown by NMR and circular dichroism to proceed with conformational adaptation.

Also suitable as modified nucleic acids are Locked Nucleic Acids (LNAs) and/or LNA analogs. In an LNA, the 2′-hydroxyl group is linked to the 4′ carbon atom of the sugar ring thereby forming a 2′-C,4′-C-oxymethylene linkage, and thereby forming a bicyclic sugar moiety. The linkage can be a methylene (—CH₂—), group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10° C.), stability towards 3′-exonucleolytic degradation and good solubility properties. Potent and nontoxic oligonucleotides containing LNAs have been described (Wahlestedt et al., Proc. Natl. Acad. Sci. U.S.A., 2000, 97, 5633-5638).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO98/39352 and WO99/14226, both of which are hereby incorporated by reference in their entirety. Exemplary LNA analogs are described in U.S. Pat. Nos. 7,399,845 and 7,569,686, both of which are hereby incorporated by reference in their entirety.

A nucleic acid can also include one or more substituted sugar moieties. Suitable polynucleotides include a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C.sub.1 to C₁₀ alkyl or C₂ to C₁₀ alkenyl and alkynyl. Also suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides include a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, and other substituents having similar properties. A suitable modification can include 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A suitable modification can include 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also referred to as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—O CH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

A nucleic acid may also include a nucleobase (also referred to as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Modified nucleobases also include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), and pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278) and are suitable base substitutions, e.g., when combined with 2′-O-methoxyethyl sugar modifications.

Quantitation of Detectable Label.

Various methods can be utilized for quantifying the presence of a detectable label, either on the detection probe, or present in a combined method with analysis of cellular markers used to define the cell being analyzed. For measuring the amount of a detection probe, or other specific binding partner that is present, a convenient method is to label with a detectable moiety, which may be a metal, fluorescent, luminescent, radioactive, enzymatically active, etc.

Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81).

Mass cytometry is a variation of flow cytometry in which probes are labeled with heavy metal ion tags rather than fluorochromes. Readout is by time-of-flight mass spectrometry This allows for the combination of many more specificities in a single samples, without significant spillover between channels. For example, see Bendall et al. (2011) Science 332 (6030): 687-696, herein specifically incorporated by reference. Nano-SIMS is an alternative method of detecting metal labels.

Multiple fluorescent or metal labels can be used on the same sample and individually detected quantitatively, permitting simultaneous multiplex analysis. Many quantitative techniques have been developed to harness the unique properties of fluorescence including: direct fluorescence measurements, fluorescence resonance energy transfer (FRET), fluorescence polarization or anisotropy (FP), time resolved fluorescence (TRF), fluorescence lifetime measurements (FLM), fluorescence correlation spectroscopy (FCS), and fluorescence photobleaching recovery (FPR) (Handbook of Fluorescent Probes and Research Chemicals, Seventh Edition, Molecular Probes, Eugene Oreg.).

Flow or mass cytometry may be used to quantitate parameters such as the presence of cell surface proteins or conformational or posttranslational modification thereof; intracellular or secreted protein, where permeabilization allows antibody (or probe) access, and the like. Both single cell multiparameter and multicell multiparameter multiplex assays, where input cell types are identified and parameters are read by quantitative imaging and fluorescence and confocal microscopy are used in the art, see Confocal Microscopy Methods and Protocols (Methods in Molecular Biology Vol. 122.) Paddock, Ed., Humana Press, 1998.

Cells.

Cells for use in the assays of the invention can be an organism, a single cell type derived from an organism, or can be a mixture of cell types. Included are naturally occurring cells and cell populations, genetically engineered cell lines, cells derived from transgenic animals, etc. Virtually any cell type and size can be accommodated. Suitable cells include bacterial, fungal, plant and animal cells. In one embodiment of the invention, the cells are mammalian cells, e.g. complex cell populations such as naturally occurring tissues, for example blood, liver, pancreas, neural tissue, bone marrow, skin, and the like. Some tissues may be disrupted into a monodisperse suspension. Alternatively, the cells may be a cultured population, e.g. a culture derived from a complex population, a culture derived from a single cell type where the cells have differentiated into multiple lineages, or where the cells are responding differentially to stimulus, and the like.

Cell types that can find use in the subject invention include stem and progenitor cells, e.g. embryonic stem cells, hematopoietic stem cells, mesenchymal stem cells, neural crest cells, etc., endothelial cells, muscle cells, myocardial, smooth and skeletal muscle cells, mesenchymal cells, epithelial cells; hematopoietic cells, such as lymphocytes, including T-cells, such as Th1 T cells, Th2 T cells, Th0 T cells, cytotoxic T cells; B cells, pre-B cells, etc.; monocytes; dendritic cells; neutrophils; and macrophages; natural killer cells; mast cells; etc.; adipocytes, cells involved with particular organs, such as thymus, endocrine glands, pancreas, brain, such as neurons, glia, astrocytes, dendrocytes, etc. and genetically modified cells thereof. Hematopoietic cells may be associated with inflammatory processes, autoimmune diseases, etc., endothelial cells, smooth muscle cells, myocardial cells, etc. may be associated with cardiovascular diseases; almost any type of cell may be associated with neoplasias, such as sarcomas, carcinomas and lymphomas; liver diseases with hepatic cells; kidney diseases with kidney cells; etc.

The cells may also be transformed or neoplastic cells of different types, e.g. carcinomas of different cell origins, lymphomas of different cell types, etc. The American Type Culture Collection (Manassas, Va.) has collected and makes available over 4,000 cell lines from over 150 different species, over 950 cancer cell lines including 700 human cancer cell lines. The National Cancer Institute has compiled clinical, biochemical and molecular data from a large panel of human tumor cell lines, these are available from ATCC or the NCI (Phelps et al. (1996) Journal of Cellular Biochemistry Supplement 24:32-91). Included are different cell lines derived spontaneously, or selected for desired growth or response characteristics from an individual cell line; and may include multiple cell lines derived from a similar tumor type but from distinct patients or sites.

Cells may be non-adherent, e.g. blood cells including monocytes, T cells, B-cells; tumor cells, etc., or adherent cells, e.g. epithelial cells, endothelial cells, neural cells, etc. In order to profile adherent cells, they may be dissociated from the substrate that they are adhered to, and from other cells, in a manner that maintains their ability to recognize and bind to probe molecules.

Such cells can be acquired from an individual using, e.g., a draw, a lavage, a wash, surgical dissection etc., from a variety of tissues, e.g., blood, marrow, a solid tissue (e.g., a solid tumor), ascites, by a variety of techniques that are known in the art. Cells may be obtained from fixed or unfixed, fresh or frozen, whole or disaggregated samples. Disaggregation of tissue may occur either mechanically or enzymatically using known techniques.

Various methods and devices exist for pre-separating component parts of the sample. These methods include filters, centrifuges, chromatographs, and other well-known fluid separation methods; gross separation using columns, centrifuges, filters, separation by killing of unwanted cells, separation with fluorescence activated cell sorters, separation by directly or indirectly binding cells to a ligand immobilized on a physical support, such as panning techniques, separation by column immunoadsorption, and separation using magnetic immunobeads.

Fixation and Permeabilization.

Aspects of the invention include “fixing” a cellular sample. The term “fixing” or “fixation” as used herein is the process of preserving biological material (e.g., tissues, cells, organelles, molecules, etc.) from decay and/or degradation. Fixation may be accomplished using any convenient protocol. Fixation can include contacting the cellular sample with a fixation reagent (i.e., a reagent that contains at least one fixative). Cellular samples can be contacted by a fixation reagent for a wide range of times, which can depend on the temperature, the nature of the sample, and on the fixative(s). For example, a cellular sample can be contacted by a fixation reagent for 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes.

A cellular sample can be contacted by a fixation reagent for a period of time in a range of from 5 minutes to 24 hours (e.g., from 10 minutes to 20 hours, from 10 minutes to 18 hours, from 10 minutes to 12 hours, from 10 minutes to 8 hours, from 10 minutes to 6 hours, from 10 minutes to 4 hours, from 10 minutes to 2 hours, from 15 minutes to 20 hours, from 15 minutes to 18 hours, from 15 minutes to 12 hours, from 15 minutes to 8 hours, from 15 minutes to 6 hours, from 15 minutes to 4 hours, from 15 minutes to 2 hours, from 15 minutes to 1.5 hours, from 15 minutes to 1 hour, from 10 minutes to 30 minutes, from 15 minutes to 30 minutes, from 30 minutes to 2 hours, from 45 minutes to 1.5 hours, or from 55 minutes to 70 minutes).

A cellular sample can be contacted by a fixation reagent at various temperatures, depending on the protocol and the reagent used. For example, in some instances a cellular sample can be contacted by a fixation reagent at a temperature ranging from −22° C. to 55° C., where specific ranges of interest include, but are not limited to: 50 to 54° C., 40 to 44° C., 35 to 39° C., 28 to 32° C., 20 to 26° C., 0 to 6° C., and −18 to −22° C. In some instances a cellular sample can be contacted by a fixation reagent at a temperature of −20° C., 4° C., room temperature (22-25° C.), 30° C., 37° C., 42° C., or 52° C.

Any convenient fixation reagent can be used. Common fixation reagents include crosslinking fixatives, precipitating fixatives, oxidizing fixatives, mercurials, and the like. Crosslinking fixatives chemically join two or more molecules by a covalent bond and a wide range of cross-linking reagents can be used. Examples of suitable cross-liking fixatives include but are not limited to aldehydes (e.g., formaldehyde, also commonly referred to as “paraformaldehyde” and “formalin”; glutaraldehyde; etc.), imidoesters, NHS (N-Hydroxysuccinimide) esters, and the like. Examples of suitable precipitating fixatives include but are not limited to alcohols (e.g., methanol, ethanol, etc.), acetone, acetic acid, etc. In some embodiments, the fixative is formaldehyde (i.e., paraformaldehyde or formalin). A suitable final concentration of formaldehyde in a fixation reagent is 0.1 to 10%, 1-8%, 1-4%, 1-2%, 3-5%, or 3.5-4.5%, including about 1.6% for 10 minutes. In some embodiments the cellular sample is fixed in a final concentration of 4% formaldehyde (as diluted from a more concentrated stock solution, e.g., 38%, 37%, 36%, 20%, 18%, 16%, 14%, 10%, 8%, 6%, etc.). In some embodiments the cellular sample is fixed in a final concentration of 10% formaldehyde. In some embodiments the cellular sample is fixed in a final concentration of 1% formaldehyde. In some embodiments, the fixative is glutaraldehyde. A suitable concentration of glutaraldehyde in a fixation reagent is 0.1 to 1%.

A fixation reagent can contain more than one fixative in any combination. For example, in some embodiments the cellular sample is contacted with a fixation reagent containing both formaldehyde and glutaraldehyde.

Permeabilization.

Aspects of the invention include “permeabilizing” a cellular sample. The terms “permeabilization” or “permeabilize” as used herein refer to the process of rendering the cells (cell membranes etc.) of a cellular sample permeable to experimental reagents such as nucleic acid probes, antibodies, chemical substrates, etc. Any convenient method and/or reagent for permeabilization can be used. Suitable permeabilization reagents include detergents (e.g., Saponin, Triton X-100, Tween-20, etc.), organic fixatives (e.g., acetone, methanol, ethanol, etc.), enzymes, etc. Detergents can be used at a range of concentrations. For example, 0.001%-1% detergent, 0.05%-0.5% detergent, or 0.1%-0.3% detergent can be used for permeabilization (e.g., 0.1% Saponin, 0.2% tween-20, 0.1-0.3% triton X-100, etc.). In some embodiments methanol on ice for at least 10 minutes is used to permeabilize.

In some embodiments, the same solution can be used as the fixation reagent and the permeabilization reagent. For example, in some embodiments, the fixation reagent contains 0.1%-10% formaldehyde and 0.001%-1% saponin. In some embodiments, the fixation reagent contains 1% formaldehyde and 0.3% saponin.

A cellular sample can be contacted by a permeabilization reagent for a wide range of times, which can depend on the temperature, the nature of the sample, and on the permeabilization reagent(s). For example, a cellular sample can be contacted by a permeabilization reagent for 24 or more hours (see storage described below), 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes. A cellular sample can be contacted by a permeabilization reagent at various temperatures, depending on the protocol and the reagent used. For example, in some instances a cellular sample can be contacted by a permeabilization reagent at a temperature ranging from −82° C. to 55° C., where specific ranges of interest include, but are not limited to: 50 to 54° C., 40 to 44° C., 35 to 39° C., 28 to 32° C., 20 to 26° C., 0 to 6° C., −18 to −22° C., and −78 to −82° C. In some instances a cellular sample can be contacted by a permeabilization reagent at a temperature of −80° C., −20° C., 4° C., room temperature (22-25° C.), 30° C., 37° C., 42° C., or 52° C.

In some embodiments, a cellular sample is contacted with an enzymatic permeabilization reagent. Enzymatic permeabilization reagents that permeabilize a cellular sample by partially degrading extracellular matrix or surface proteins that hinder the permeation of the cellular sample by assay reagents. Contact with an enzymatic permeabilization reagent can take place at any point after fixation and prior to target detection. In some instances the enzymatic permeabilization reagent is proteinase K, a commercially available enzyme. In such cases, the cellular sample is contacted with proteinase K prior to contact with a post-fixation reagent (described below). Proteinase K treatment (i.e., contact by proteinase K; also commonly referred to as “proteinase K digestion”) can be performed over a range of times at a range of temperatures, over a range of enzyme concentrations that are empirically determined for each cell type or tissue type under investigation. For examples, a cellular sample can be contacted by proteinase K for 30 or less minutes, 25 or less minutes, 20 or less minutes, 15 or less minutes, 10 or less minutes, 5 or less minutes, or 2 or less minutes. A cellular sample can be contacted by 1 ug/ml or less, 2 ug/m or less I, 4 ug/ml or less, Bug/ml or less, 10 ug/ml or less, 20 ug/ml or less, 30 ug/ml or less, 50 ug/ml or less, or 100 ug/ml or less proteinase K. A cellular sample can be contacted by proteinase K at a temperature ranging from 2° C. to 55° C., where specific ranges of interest include, but are not limited to: 50 to 54° C., 40 to 44° C., 35 to 39° C., 28 to 32° C., 20 to 26° C., and 0 to 6° C. In some instances a cellular sample can be contacted by proteinase K at a temperature of 4° C., room temperature (22-25° C.), 30° C., 37° C., 42° C., or 52° C. In some embodiments, a cellular sample is not contacted with an enzymatic permeabilization reagent. In some embodiments, a cellular sample is not contacted with proteinase K.

Contact of a cellular sample with at least a fixation reagent and a permeabilization reagent results in the production of a fixed/permeabilized cellular sample.

Nuclease Inhibition.

Aspects of the invention include contacting a cellular sample with a nuclease inhibitor during hybridization steps, particularly during binding of the target specific oligonucleotide pair to RNA molecules present in the cell. As used herein, a “nuclease inhibitor” is any molecule that can be used to inhibit nuclease activity within the cellular sample such that integrity of the nucleic acids within the cells of the cellular sample is preserved. In other words, degradation of the nucleic acids within the cells of the cellular sample by nuclease activity is inhibited by contacting the cellular sample with a nuclease inhibitor.

In some embodiments, the nuclease inhibitor is an RNase inhibitor (i.e., the inhibitor inhibits RNase activity). Examples of suitable commercially available nuclease inhibitors include, protein and non-protein based inhibitors, e.g. vanadyl ribonucleoside complexes, Oligo(vinylsulfonic Acid) (OVS), 2.5%, aurintricarboxylic acid (ATA); Diethyl Pyrocarbonate (DEPC); RNAsecure™ Reagent from Life Technologies; and the like) and protein based inhibitors (e.g., ribonuclease inhibitor from EMD Millipore; RNaseOUT™ Recombinant Ribonuclease Inhibitor, SUPERaseIn™, ANTI-RNase, and RNase Inhibitor from Life Technologies; RNase Inhibitor and Protector RNase Inhibitor from Roche; RNAsin from Promega, and the like). Nuclease inhibitors can be used at a range of concentrations as recommended by their commercial sources.

Marker Detection Reagents.

Aspects of the invention may include contacting the cells in a sample with a detection reagent in order to profile cells simultaneously for markers in addition to the target nucleic acids. Such methods are particularly useful in detecting the phenotype of cells in complex populations, e.g. populations of immune cells, populations of neural cells, complex biopsy cell populations, and the like. The term “marker detection reagent” as used herein refers to any reagent that specifically binds to a target marker (e.g., a target protein of a cell of the cellular sample) and facilitates the qualitative and/or quantitative detection of the target protein. The terms “specific binding,” “specifically binds,” and the like, refer to the preferential binding to a molecule relative to other molecules or moieties in a solution or reaction mixture. In some embodiments, the affinity between detection reagent and the target protein to which it specifically binds when they are specifically bound to each other in a binding complex is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less. “Affinity” refers to the strength of binding, increased binding affinity being correlated with a lower K_{d}.

In some embodiments, a protein detection reagent includes a label or a labeled binding member. A “label” or “label moiety” is any moiety that provides for signal detection and may vary widely depending on the particular nature of the assay, and includes any of the labels suitable for use with the oligonucleotide detection probe, described above.

In some instances, a protein detection reagent is a polyclonal or monoclonal antibody or a binding fragment thereof (i.e., an antibody fragment that is sufficient to bind to the target of interest, e.g., the protein target). Antibody fragments (i.e., binding fragments) can be, for example, monomeric Fab fragments, monomeric Fab′ fragments, or dimeric F(ab)′₂ fragments. Also within the scope of the term “antibody or a binding fragment thereof” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies.

Markers of interest include cytoplasmic, cell surface or secreted biomolecules, frequently biopolymers, e.g. polypeptides, polysaccharides, polynucleotides, lipids, etc. Where the marker is a protein the detection may include states of phosphorylation, glycosylation, and the like as known in the art.

### Methods of Use

Multiplexed assays as demonstrated here save time and effort, as well as precious clinical material, and permit analysis of genetic events such as copy number amplification, RNA expression etc. at a single cell level. More importantly, the ability to simultaneously assess multiple concurrent molecular events within the same cells can provide entirely new opportunities to elucidate the intricate networks of interactions within cells. Multiplexed analysis can be used to measure and quantify the balance between genetic interactions for an improved understanding of cellular functions.

Aspects of the invention include methods of assaying a cellular sample for the presence of a target nucleic acid (e.g., deoxyribonucleic acid, ribonucleic acid) at the single cell level, usually a plurality of target nucleic acids at a single cell level. The analysis can be combined with analysis of additional markers that define cells within the population, e.g. protein markers.

As such, methods of the invention are methods of evaluating the amount (i.e., level) of a target nucleic acid in a cell of a cellular sample. In some embodiments, methods of the invention are methods of evaluating whether a target nucleic acid is present in a sample, where the detection of the target nucleic acid is qualitative. In some embodiments, methods of the invention are methods of evaluating whether a target nucleic acid is present in a sample, where the detection of the target nucleic acid is quantitative. The methods can include determining a quantitative measure of the amount of a target nucleic acid in a cell of a cellular sample. In some embodiments, quantifying the level of expression of a target nucleic acid includes comparing the level of expression of one nucleic acid to the level of expression of another nucleic acid in order to determine a relative level of expression. In some embodiments, the methods include determining whether a target nucleic acid is present above or below a predetermined threshold in a cell of a cellular sample. As such, when the detected signal is greater than a particular threshold (also referred to as a “predetermined threshold”), the amount of target nucleic acid of interest is present above the predetermined threshold in the cell of a cellular sample. When the detected signal is weaker than a predetermined threshold, the amount of target nucleic acid of interest is present below the predetermined threshold in the cell of a cellular sample.

The term “cellular sample,” as used herein means any sample containing one or more individual cells in suspension at any desired concentration. For example, the cellular sample can contain 10¹¹ or less, 10¹⁰ or less, 10⁹ or less, 10⁸ or less, 10⁷ or less, 10⁶ or less, 10⁵ or less, 10⁴ or less, 10³ or less, 500 or less, 100 or less, 10 or less, or one cell per milliliter. The sample can contain a known number of cells or an unknown number of cells. Suitable cells include eukaryotic cells (e.g., mammalian cells) and/or prokaryotic cells (e.g., bacterial cells or archaeal cells).

In practicing the methods of the invention, the cellular sample can be obtained from an in vitro source (e.g., a suspension of cells from laboratory cells grown in culture) or from an in vivo source (e.g., a mammalian subject, a human subject, etc.). In some embodiments, the cellular sample is obtained from an in vitro source. In vitro sources include, but are not limited to, prokaryotic (e.g., bacterial, archaeal) cell cultures, environmental samples that contain prokaryotic and/or eukaryotic (e.g., mammalian, protest, fungal, etc.) cells, eukaryotic cell cultures (e.g., cultures of established cell lines, cultures of known or purchased cell lines, cultures of immortalized cell lines, cultures of primary cells, cultures of laboratory yeast, etc.), tissue cultures, and the like.

In some embodiments, the sample is obtained from an in vivo source and can include samples obtained from tissues (e.g., cell suspension from a tissue biopsy, cell suspension from a tissue sample, etc.) and/or body fluids (e.g., whole blood, fractionated blood, plasma, serum, saliva, lymphatic fluid, interstitial fluid, etc.). In some cases, cells, fluids, or tissues derived from a subject are cultured, stored, or manipulated prior to evaluation. In vivo sources include living multi-cellular organisms and can yield non-diagnostic or diagnostic cellular samples.

Cellular samples can be obtained from a variety of different types of subjects. In some embodiments, a sample is from a subject within the class mammalia, including e.g., the orders carnivore (e.g., dogs and cats), rodentia (e.g., mice, guinea pigs, and rats), lagomorpha (e.g. rabbits) and primates (e.g., humans, chimpanzees, and monkeys), and the like. In certain embodiments, the animals or hosts, i.e., subjects (also referred to herein as patients) are humans.

Aspects of the invention may include contacting the cellular sample with a “stimulating agent”, also referred to herein as a “stimulator.” By stimulating agent it is meant any compound that affects at least one cellular activity or that alters the cellular steady state (i.e., induced or reduced in abundance or activity). Contacting a cellular sample with a stimulating agent can be used to ascertain the cellular response to the agent. By “effective amount” of a stimulating agent, it is meant that a stimulating agent is present in an amount to affect at least one cellular activity that alters the cellular steady state (i.e., induced or reduced in abundance or activity). A stimulating agent can be provided as a powder or as a liquid. As such, a stimulating agent can include various compounds and formulations, such as intracellular signal inducing and immunomodulatory agents. Examples include small molecule drugs as well as peptides, proteins, lipids carbohydrates and the like. Of particular interest are compounds such as peptide hormones, chemokines, cytokines, e.g. type I interferons (e.g., IFN-α, IFN-β), interleukins (e.g., interleukin-2 (IL-2), IL-4, IL-6, IL-7, IL-10, IL-12, IL-15, IL-21), tumor necrosis factor alpha (TNF-α), gamma interferon (IFN-γ), transforming growth factor R, and the like.

### Target Nucleic Acid Detection

The subject methods are methods of assaying for the presence of a target nucleic acid. As such, the subject methods are methods (when a target nucleic acid is present in a cell of a cellular sample) of detecting the target nucleic acid, producing a signal in response to target nucleic acid detection, and detecting the produced signal. The signal produced by a detected target nucleic acid can be any detectable signal (e.g., a fluorescent signal, an amplified fluorescent signal, a chemiluminescent signal, etc.)

Aspects of the invention include methods of detecting a target nucleic acid (i.e., target nucleic acid detection). In some embodiments, the cellular sample is contacted with a nucleic acid detection agent. As used herein, the term “nucleic acid detection agent” means any reagent that can specifically bind to a target nucleic acid. For example, suitable nucleic acid detection agents can be nucleic acids (or modified nucleic acids) that are at least partially complementary to and hybridize with a sequence of the target nucleic acid. In some embodiments, the nucleic acid detection agent includes a probe or set of probes (i.e., probe set), each of which specifically binds (i.e., hybridizes to) a sequence (i.e., target sequence) of the target nucleic acid.

In some embodiments, a method is provided for determining the abundance of a target nucleic acid in a single cell, the method comprising contacting a fixed and permeabilized cell with at least one pair of oligonucleotide primers under conditions permissive for specific hybridization, wherein each oligonucleotide in the pair comprises: a target binding region that hybridizes to the target nucleic acid; a spacer region that does not bind to the target nucleic acid or to any region of a padlock probe; and an PLAYR 1 or PLAYR 2 region that specifically binds to the padlock probe, wherein the padlock probe comprises two polynucleotides, a backbone and an insert, and wherein the PLAYR 1 or PLAYR 2 region binds to both insert and backbone; washing the cells free of unbound primers; contacting the cells with backbone and insert polynucleotides under conditions permissive for specific hybridization; washing the cells free of unbound backbone insert; performing a ligation reaction, in which bound backbone insert polynucleotides are ligated to generate a circle; amplifying the ligated backbone/insert circle by rolling circle amplification; hybridizing detection primers to the amplified circle; and quantitating the level of bound detection primers to determine the abundance of the target nucleic acid.

In some embodiments of the invention, PLAYR is used in combination with cytometry gating on specific cell populations, as defined by other cellular parameters measured simultaneously, for example in combination with antibody staining and mass cytometry or FACS to define a subpopulation of interest. In such embodiments, a complex cell population may be analyzed, e.g. a biopsy or blood sample potentially including immune cells, progenitor or stem cells, cancer cells, etc. For example, a method is provided for determining the abundance of one or more target nucleic acids in a defined cell type within a complex cell population, where the quantification of detection probes is combined with detection of cellular markers, including without limitation protein markers, that serve to define the cell type of interest.

In other embodiments, the methods of the invention are used for multiplexed detection and quantification of specific splice variants of mRNA transcripts in single cells.

In yet another embodiment, the methods of the invention are combined with Proximity Ligation Assay (PLA) for the simultaneous detection and quantification of nucleic acid molecules and protein-protein interactions.

With prior denaturation of endogenous cellular DNA (by heat, enzymatic methods, or any other suitable procedure), the technology is modified for the detection of specific DNA sequences (genotyping of single cells). In this adaptation, the technology enables the quantification of gene copy number variations as well as the detection of genomic translocation/fusion events.

Signal detection and quantitation can be carried out using any instrument (e.g., liquid assay device) that can measure the fluorescent, luminescent, light-scattering or colorimetric signal(s) output from the subject methods. In some embodiments, the signal resulting from the detection of a target nucleic acid is detected by a flow cytometer. In some embodiments, a liquid assay device for evaluating a cellular sample for the presence of the target nucleic acid is a flow cytometer, e.g. mass cytometer, FACS, MACS, etc. As such, in some instances, the evaluation of whether a target nucleic acid is present in a cell of a cellular sample includes flow cytometrically analyzing the cellular sample. In flow cytometry, cells of a cellular sample are suspended in a stream of fluid, which is passed, one cell at a time, by at least one beam of light (e.g., a laser light of a single wavelength). A number of detectors, including one or more fluorescence detectors, detect scattered light as well as light emitted from the cellular sample (e.g., fluorescence). In this way, the flow cytometer acquires data that can be used to derive information about the physical and chemical structure of each individual cell that passes through the beam(s) of light. If a signal specific to the detection of a target nucleic acid is detected in a cell by the flow cytometer, then the target nucleic acid is present in the cell. In some embodiments, the detected signal is quantified using the flow cytometer.

The readout may be a mean, average, median or the variance or other statistically or mathematically-derived value associated with the measurement. The readout information may be further refined by direct comparison with the corresponding reference or control, e.g. by reference to a standard polynucleotide sample, housekeeping gene expression, etc. The absolute values obtained for under identical conditions may display a variability that is inherent in live biological systems.

In certain embodiments, the obtained data is compared to a single reference/control profile to obtain information regarding the phenotype of the cell being assayed. In yet other embodiments, the obtained data is compared to two or more different reference/control profiles to obtain more in depth information regarding the phenotype of the cell. For example, the obtained data may be compared to a positive and negative controls to obtain confirmed information regarding whether a cell has a phenotype of interest.

### Utility

The methods, devices, compositions and kits of the invention find use in a variety of different applications. Methods of the invention are methods of evaluating cells of a cellular sample, where the target nucleic acid may or may not be present. In some cases, it is unknown prior to performing the assay whether a cell of the cellular sample expresses the target nucleic acid. In other instances, it is unknown prior to performing the assay whether a cell of the cellular sample expresses the target nucleic acid in an amount (or relative amount, e.g., relative to another nucleic acid or relative to the amount of the target nucleic acid in a normal cell) that is greater than (exceeds) a predetermined threshold amount (or relative amount). In such cases, the methods are methods of evaluating cells of a cellular sample in which the target nucleic acid of interest may or may not be present in an amount that is greater than (exceeds) or below than a predetermined threshold. In some embodiments, the methods of the invention can be used to determine the expression level (or relative expression level) of a nucleic acid in individual cell(s) of a cellular sample, usually a multiplex analysis of multiple nucleic acids in a cell. Optionally additional markers such as proteins are also analyzed.

The methods of the invention can be used to identify specific cells in a sample as aberrant or non-aberrant. For example, some mRNAs are known to be expressed above a particular level, or relative level, (i.e., above a predetermined threshold) in aberrant cells (e.g., cancerous cells). Thus, when the level (or relative level) of signal (as detected using the subject methods) for a particular target nucleic acid (e.g., mRNA) of a cell of the cellular sample indicates that the level (or relative level) of the target nucleic acid is equal to or greater than the level (or relative level) known to be associated with an aberrant cell, then the cell of the cellular sample is determined to be aberrant. To the contrary, some mRNAs (and/or miRNAs) are known to be expressed below a particular level, or relative level, (i.e., below a predetermined threshold) in aberrant cells (e.g., cancerous cells). Thus, when the level (or relative level) of signal (as detected using the subject methods) for a particular target nucleic acid of a cell of the cellular sample indicates that the level (or relative level) of the target nucleic acid is equal to or less than the level (or relative level) known to be associated with an aberrant cell, then the cell of the cellular sample is determined to be aberrant. Therefore, the subject methods can be used to detect and count the number and/or frequency of aberrant cells in a cellular sample. Any identified cell of interest can be profiled for additional information with respect to protein or other markers.

In some instances, it is unknown whether the expression of a particular target nucleic acid varies in aberrant cells and the methods of the invention can be used to determine whether expression of the target nucleic varies in aberrant cells. For example, a cellular sample known to contain no aberrant cells can be evaluated and the results can be compared to an evaluation of a cellular sample known (or suspected) to contain aberrant cells.

In some instances, an aberrant cell is a cell in an aberrant state (e.g., aberrant metabolic state; state of stimulation; state of signaling; state of disease; e.g., cell proliferative disease, cancer; etc.). In some instances, an aberrant cell is a cell that contains a prokaryotic, eukaryotic, or viral pathogen. In some cases, an aberrant pathogen-containing cell (i.e., an infected cell) expresses a pathogenic mRNA or a host cell mRNA at a level above cells that are not infected. In some cases, such a cell expresses a host cell mRNA at a level below cells that are not infected.

In embodiments that employ a flow cytometer to flow cytometrically analyze the cellular sample, evaluation of cells of the cellular sample for the presence of a target nucleic acid can be accomplished quickly, cells can be sorted, and large numbers of cells can be evaluated. Gating can be used to evaluate a selected subset of cells of the cellular sample (e.g., cells within a particular range of morphologies, e.g., forward and side-scattering characteristics; cells that express a particular combination of surface proteins; cells that express particular surface proteins at particular levels; etc.) for the presence or the level (or relative level) of expression of a target nucleic acid.

In some embodiments, the methods are methods of determining whether an aberrant cell is present in a diagnostic cellular sample. In other words, the sample has been obtained from or derived from an in vivo source (i.e., a living multi-cellular organism, e.g., mammal) to determine the presence of a target nucleic acid in one or more aberrant cells in order to make a diagnosis (i.e., diagnose a disease or condition). Accordingly, the methods are diagnostic methods. As the methods are “diagnostic methods,” they are methods that diagnose (i.e., determine the presence or absence of) a disease (e.g., cancer, circulating tumor cell(s), minimal residual disease (MRD), a cellular proliferative disease state, viral infection, e.g., HIV, etc.) or condition (e.g., presence of a pathogen) in a living organism, such as a mammal (e.g., a human). As such, certain embodiments of the present disclosure are methods that are employed to determine whether a living subject has a given disease or condition (e.g., cancer, circulating tumor cell(s), minimal residual disease (MRD), a cellular proliferative disease state, a viral infection, presence of a pathogen, etc.). “Diagnostic methods” also include methods that determine the severity or state of a given disease or condition based on the level (or relative level) of expression of at least one target nucleic acid.

In some embodiments, the methods are methods of determining whether an aberrant cell is present in a non-diagnostic cellular sample. A non-diagnostic cellular sample is a cellular sample that has been obtained from or derived from any in vitro or in vivo source, including a living multi-cellular organism (e.g., mammal), but not in order to make a diagnosis. In other words, the sample has been obtained to determine the presence of a target nucleic acid, but not in order to diagnose a disease or condition. Accordingly, such methods are non-diagnostic methods.

The results of such analysis may be compared to results obtained from reference compounds, concentration curves, controls, etc. The comparison of results is accomplished by the use of suitable deduction protocols, artificial evidence systems, statistical comparisons, etc. In particular embodiments, the method described above may be employed in a multiplex assay in which a heterogeneous population of cells is labeled with a plurality of distinguishably labeled binding agents.

A database of analytic information can be compiled. These databases may include results from known cell types, references from the analysis of cells treated under particular conditions, and the like. A data matrix may be generated, where each point of the data matrix corresponds to a readout from a cell, where data for each cell may comprise readouts from multiple labels. The readout may be a mean, median or the variance or other statistically or mathematically derived value associated with the measurement. The output readout information may be further refined by direct comparison with the corresponding reference readout. The absolute values obtained for each output under identical conditions will display a variability that is inherent in live biological systems and also reflects individual cellular variability as well as the variability inherent between individuals.

### Kits

Also provided by the present disclosure are kits for practicing the method as described above. The subject kit contains reagents for performing the method described above and in certain embodiments may contain a plurality of probes and primers, including for example at least one pair of target specific oligonucleotide primers; a corresponding insert and backbone for a padlock probe; and a detection probe optionally labeled with a detectable moiety. The kit may also contain a reference sample to which results obtained from a test sample may be compared.

In addition to above-mentioned components, the subject kit may further include instructions for using the components of the kit to practice the methods described herein. The instructions for practicing the subject method are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging), etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate. In addition to above-mentioned components, the subject kit may include software to perform comparison of data.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. All technical and scientific terms used herein have the same meaning as commonly understood to one of ordinary skill in the art to which this invention belongs unless clearly indicated otherwise.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

The invention will now be more fully described in association with some examples which are not to be construed as limiting for the invention.

### Example 1

Measurements of gene expression are a fundamental tool to understand how genetic networks coordinately function in normal cells and tissues and how they malfunction in disease. The most commonly used methods (e.g. qPCR, microarrays or RNA-seq) are bulk assays that only measure the average expression in a sample. As such they cannot detect expression signature that are specific to a small population of cells within a complex sample.

Recently, microfluidics-based methods have been developed to perform RNAseq in single cells by physically separating the cells and running separate sequencing reactions. This is a powerful genome-wide technique but only a few hundred cells can be analyzed and the costs of the procedure are very high, impeding routine applications.

To overcome these limitations, we developed a method to simultaneously quantify ˜20 RNAs of interest in single cells with the following major advantages: (a) hundreds of cells can be analyzed per second with a conventional flow-cytometer or with a mass-cytometer. The technology is thus well-suited for the analysis of complex samples comprising large numbers of cells; and (b) RNAs can be detected simultaneously with proteins and other cellular antigens. The functional state of each cell can thus be analyzed e.g. with antibodies directed against intracellular phosphorylation sites.

Our technology uses pairs of short oligonucleotide probes that specifically hybridize to adjacent regions of the target RNA. Each probe in the pair is extended with specific sequences that jointly act as a template for the hybridization of a second set of oligonucleotides, which are added in a subsequent step. If hybridized correctly, these additional oligonucleotides can be ligated to form a continuous single-stranded DNA circle. This circular product is then amplified using Rolling Circle Amplification, which produces a molecule that contains hundreds of concatenated complementary copies of the original single-stranded DNA circle. This RCA product can then be detected with a suitably-labeled complementary oligonucleotide. In this approach, a high level of specificity results from the fact that both of the primary probes need to hybridize to adjacent locations of a target RNA for the amplification reaction to take place. Non-specific, off-target binding of a single probe does not produce any signal.

The RNA Ligation Assay (PLAYR) of the invention enables the quantitation of specific RNAs in single cells by detecting the simultaneous binding of two probes to adjacent regions of a RNA target. The proximal binding of such two probes is converted by a number of steps into a linear, single-stranded DNA product, which can be bound by hundreds of suitably labeled detection oligonucleotides and the resulting signal is measured with an appropriate analysis platform. The technology is very specific despite the fairly short target hybridization sequence (˜20 nucleotides) of the individual single probes. This high specificity stems from the fact that any off-target binding of a single probe does not generate any signal. In contrast, the binding of two probes in close proximity, which only happens on the intended target, leads to greatly amplified and easily detectable signals.

The protocol comprises the following steps (see FIG. 1).

PLAYR Probe pair hybridization: each probe consists of: i) a sequence complementary to the target RNA, ˜20 bp in length; ii) a ˜10 bp spacer, iii) a synthetic sequence, either PLAYR 1 or PLAYR 2. The two probes in a pair are designed to hybridize to adjacent regions of the target (˜3-50 bp distance between binding sites on target). One probe is extended with the PLAYR 1 sequence, while the other is extended with PLAYR 2. When brought into proximity by binding of both probes to an intended target, PLAYR 1 and PLAYR 2 combined serve as a template for the hybridization of two subsequently added oligonucleotides.

Backbone/Insert hybridization: the two probes added after the initial target binding of the PLAYR probes are termed Backbone and Insert, respectively. The Insert consists of two adjacent regions, which are complementary to PLAYR 1 and PLAYR 2, respectively. The Backbone is also complementary to both PLAYR 1 and PLAYR 2 but the hybridization regions are located at the two ends of the oligo, separated by a spacer. When two PLAYR probes bind to adjacent regions of a target RNA, the PLAYR 1 and PLAYR 2 sequences serve as template for the hybridization of Backbone and Insert which, by virtue of their designed sequences, form a circular, single-stranded DNA structure.

Ligation: the Insert and the Backbone termini are ligated by the enzyme T4 DNA ligase, resulting in a continuous circle of DNA consisting of the Backbone and Insert. This step crucially enforces the specificity of the system because the termini can be ligated only if the Backbone and Insert are correctly hybridized to both the PLAYR 1 and PLAYR 2 sequences. The fact that two ligation events are necessary for the formation of a circular product makes PLAYR template-independent ligation of Backbone and Insert virtually impossible, increasing the specificity of the approach.

Amplification: the enzyme phi29 polymerase, using one of the free termini of the PLAYR probes as a primer and the DNA circle as a template, produces hundreds to thousands of concatenated complementary copies of the DNA circle in a process termed Rolling Circle Amplification (RCA). This great degree of amplification produces RCA products that can be detected and counted individually using a microscope and lead to detectable increases in signal intensity on a per-cell basis when analyzed by flow or mass cytometry.

Detection: a labeled detection oligo, which is complementary to a sequence that is present hundreds to thousands of times in the linear RCA product, is added to the sample and unbound detection oligos are washed away. The resulting signal can then be measured with an appropriate detection platform depending on how the oligo was labeled. For analysis by microscopy or flow cytometry, fluorescently labeled detection oligos are used, while metal-conjugated oligos enable mass cytometric or nano-SIMS analyses. The detection oligo is complementary to the RCA product, which is itself a copy of the DNA circle originally formed by the Backbone and Insert. Therefore, the sequence of the detection oligo is identical to a region of the Backbone, the Insert, or a combination of the two.

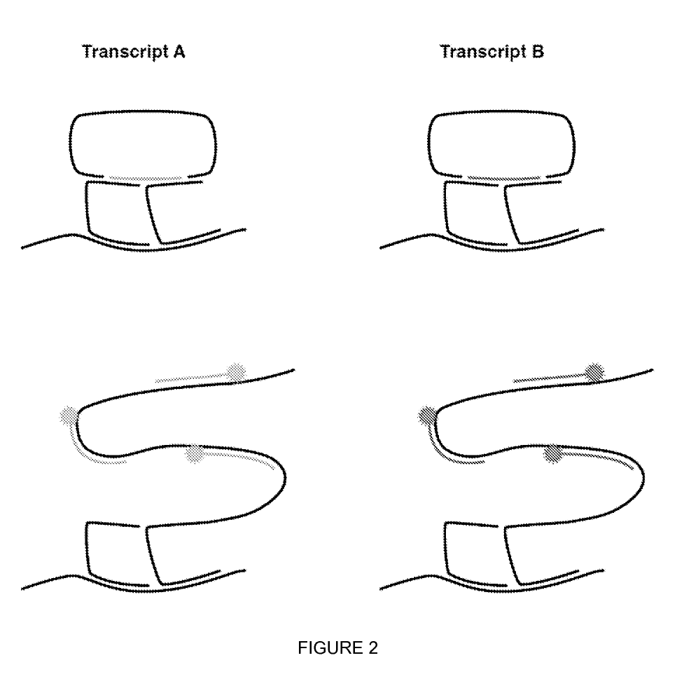

The technology can easily be multiplexed by varying the synthetic sequences comprising the signal amplification system. This is most effectively achieved by designing different PLAYR 1 and PLAYR 2 sequences and complementary Inserts (FIG. 2). Specific PLAYR 1/PLAYR 2 sequences are then attached to one or several different PLAYR probe pairs that are specific for a given transcript. This way, the PLAYR 1/PLAYR 2 sequences barcode for the RNA target bound by the PLAYR probe pairs. The Backbone sequence can be kept constant by only varying the portion of PLAYR 1 and PLAYR 2 that are complementary to the Insert, while keeping the Backbone-complementary portions constant. This minimizes differences in the amount of RCA product that is generated while making the products different enough for template-specific detection.

PLAYR Probe Design.

To ensure the specificity of the technology and to reduce the variability between different PLAYR probes for the same or different transcripts, a number of parameters were considered when designing the probes. The melting temperatures for the hybridization to the RNA targets were similar for all probes, typically in the range of 58-62 degrees Celsius. The hybridization to the target typically spanned 18-25 bp, the GC content of all probes was kept below 70%, and probes did not contain more than three consecutive guanine nucleobases. Furthermore, the probes were typically designed such that they target constitutive exons and do not span exon boundaries. Lastly, BLAST searches were run with the designed PLAYR probes to ensure that there is no cross-reactivity with other transcripts that might be expressed in the samples to be analyzed.

PLAYR can Detect Specific RNAs in Single Cells.

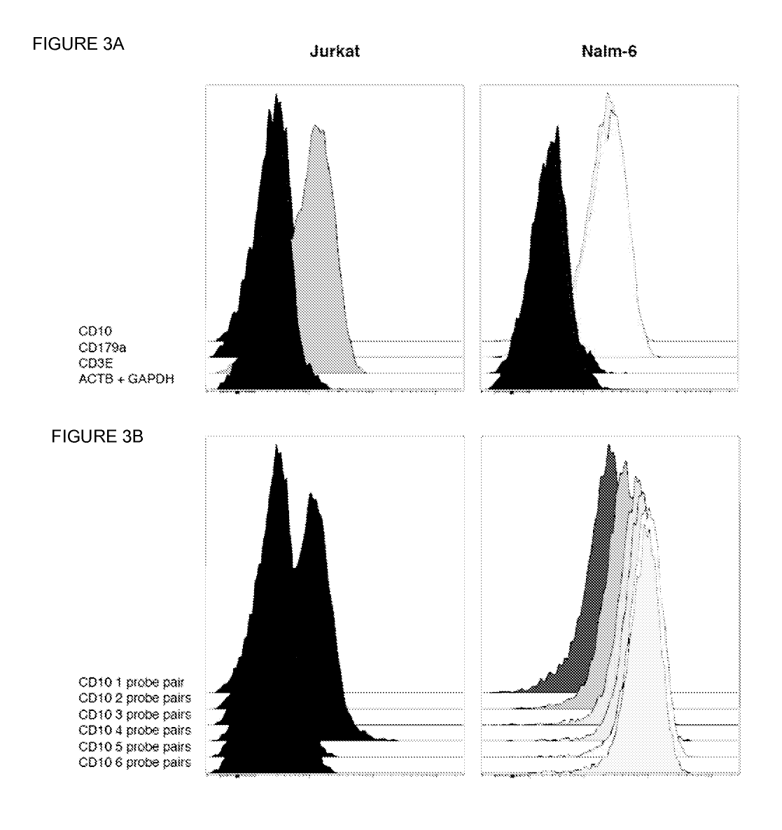

The following negative controls show the PLAYR signal to be specific for the target RNA (FIG. 3A): If two probes targeting two different genes are used, no signal is obtained. Therefore, for a signal to be generated, the two probes must bind in close proximity on the same target nucleic acid. Probes against a specific transcript do not produce signals when incubated with cell types that are known not to express the transcript. Using multiple probe pairs against the same transcript leads to an increase in signal

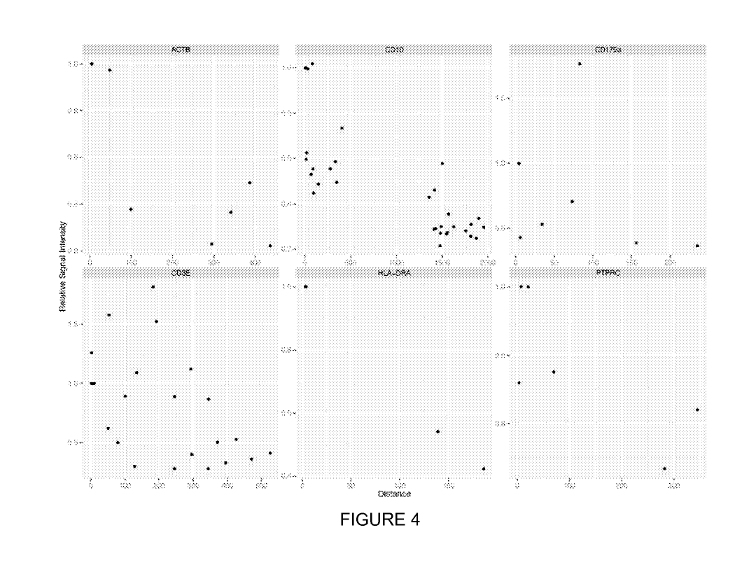

The PLAYR signal can be increased by using multiple probe sets directed against the same transcript (FIG. 3B). Interestingly, the signal increase can be more than additive, because a probe in one set can also pair to a second probe in a different set on the same transcript, even though the target regions of the two sets are not immediately adjacent. This spatial proximity of bound probes despite their distant binding sites can be explained by the folded secondary structure of RNA molecules in three dimensions. Indeed, we have observed that pairs of distant probes on the same transcript can still give rise to a signal. Accordingly, there is a strong, albeit not perfect, inverse relationship between the strength of the signal and the distance in the target hybridization regions of the two probes in a pair (FIG. 4). Besides increasing the sensitivity, using more than one probe pair per transcript can also make results for individual genes more reproducible since signals for individual transcripts are less affected by sequence accessibility and alternative splicing.

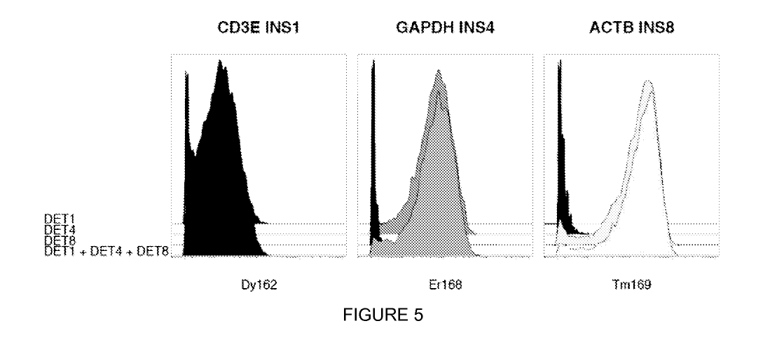

Multiple transcript can be detected simultaneously in single cells. Specific PLAYR 1/PLAYR 2 sequences can be attached to any transcript-targeting sequence and can be used to barcode for a targeted transcript after RCA. Using this strategy, multiple targets can be detected simultaneously within individual cells (FIG. 5). In such a system, the number of targets that can be detected within the same cell is only limited by the number of reporters that can be conjugated to detection oligonucleotides and analyzed simultaneously with a given platform (typically 4-5 with fluorescence reporters or 30-40 with metal reporters). We have designed PLAYR 1/PLAYR 2 probe sequences that hybridize to the same Backbone but are complementary to different Inserts. These systems were designed to have highly similar thermodynamic properties, i.e. both PLAYR arms have identical melting temperatures across all insert systems. This ensures that all systems are equally efficient templates for the formation of RCA products and the detection thereof. At the same time, all Inserts are substantially different in their sequence and have a longest common substring of 4 bases. This ensures that the resulting RCA products barcode for the detected transcript and can be detected with different detection oligonucleotides conjugated to corresponding reporters. Using this strategy, we have detected several genes simultaneously without any compromise in signal intensity compared to the detection of the same transcripts one at a time.

### Materials and Methods

The protocol comprises the following steps, which are described in more detail in the following paragraphs: PLAYR probe design, Cell fixation/permeabilization, Probes hybridization, Stringency wash, Backbone/Insert hybridization, Ligation, Amplification, Detection.

The carrier solution for most of the protocol is PBSTR (PBS+0.1% Tween+Promega RNAsin (1 uL/10 mL)). The reaction volume in each step was typically 50 μL, which is appropriate for 10⁴-1×10⁶ cells per sample. The number of cells in a sample has a strong effect on the amount of signals and should be the same in all samples to enable relative transcript quantification across samples. It is therefore important that the number of cells be consistent across samples for the results to be comparable.

Probe design. Whenever possible, probes are designed so that they target constitutive exons within transcripts as determined by public databases. When using multiple PLAYR probe pairs per transcript, different pairs are typically designed to target different exons and not to span exon boundaries to minimize variability in the measurements introduced by alternative splicing and varying sequence accessibility. All probes used for a given experiment have highly similar DNA/RNA melting temperatures, usually 60+/−2 degrees Celsius for the target specific hybridization. Also, the RNA targeting sequences are of similar length for all probes, typically 18-25 base pairs and have a GC content between 30-70%. Finally, suitable probes are BLAST search to avoid cross-hybridization to other transcripts that may be present in the samples. The RNA targeting sequences are then extended by a 10 base pair spacer, typically poly A, and a corresponding PLAYR 1 or PLAYR 2 sequence.

Cell fixation/permeabilization. We use the standard protocol described in (Krutzik & Nolan (2003) Cytometry. Part A: the journal of the International Society for Analytical Cytology, 55A(2), pp. 61-70), with minor modifications. Briefly: Resuspend live cells to a density of 1 million/mL in growth medium without FBS. Add paraformaldehyde (PFA) to a final concentration of 1.6% and incubate for 10 minutes at room temperature with gentle agitation. Centrifuge cells at 300 g for 5 minutes and aspirate supernatant. Vortex cells in the residual volume and add ice-cold methanol drop-wise with continuous vortexing. Incubate for 10 minutes on ice. The cells can be stored in methanol for months as long as the temperature is below 4 C.

Probe hybridization. Once the cells are transferred from methanol back in an aqueous phase, RNA starts to be degraded by endogenous RNAses present in the cells, which survive the fixation/permeabilization procedure. We have experimented with a number of different inhibitors and we are currently using the following cocktail:
Promega RNAsin (1 μL/mL)
Vanadyl ribonucleoside complexes (VRC, 20 mM)
Oligo(vinylsulfonic Acid) (OVS, 2.5%)(Smith et al. (2003) J Biochem, 278(23), pp.20934-20938)
   RNAse inhibition is necessary and greatly improves the results, although no single inhibitor is absolutely required per se. The amount of RNAse activity, and thus the need for inhibition, varies in different cell types.

The oligonucleotide probes are typically used at a concentration of 100 nM and they need to be denatured at 90 C for 5 minutes and then chilled on ice before being added to the cells. This step is critical, failure to denature the probes will result in very high background. Moreover, if this step is omitted, it is possible to get signals even for probe pairs that do not target the same gene.

The hybridization buffer was composed as follows: RNAse inhibitor cocktail, as described above, 3×SSC, 1% Tween, Salmon Sperm DNA (100 μg/mL). Starting from a 100 μM stock of probes: Dilute the probes 1:50 in water. Heat up the probes at 90 C for 5 minutes then chill on ice. Add 2.5 uL of probes to 47.5 of cells that have already been resuspended in hybridization buffer. This makes the final concentration of the probes 100 nM (1:1000 dilution of the 100 μM stock). Incubate for 60 min. at 40 C. Wash three times with PBSTR, at a temperature from 30-40° C., a salt concentration from 0.5×-5×SSC, and formamide from 0-50%.

Stringency wash. This washing step after the hybridization markedly improves the signal/noise ratio. The wash buffer was as follows: 5×SSC, 0.1% Tween, RNAsin (1 μL/mL). The cells were incubated for 20 min. at 40 C on a shaker in 50 μL of the wash buffer, and washed twice with PBSTR.

Incubation with Backbone/Insert. Hybridization buffer: 1×SSC+0.1% Tween. Backbone concentration: 100 nM. Insert concentration: 100 nM. RNAsin: 1 μL/mL. Incubate at 37 C for 30 minutes, reaction volume 50 μL. Wash twice with PBSTR.

Ligation. The Backbone/Insert are ligated using T4 DNA ligase. Reaction buffer: as recommended by vendor. Enzyme: 0.005 U/μL, RNAsin: 1 μL/mL. Incubate at 37 C for 30 minutes, reaction volume 50 μL. Wash twice with PBSTR.

Amplification. The DNA circles are amplified using phi29 DNA polymerase. Reaction buffer: as recommended by vendor, Enzyme: 0.125 U/μL, RNAsin: 1 μL/mL. Incubate at 30 C for 120 minutes to overnight, reaction volume 50 μL. Wash twice with PBSTR.

Detection. Hybridization buffer: 1×SSC+0.1% Tween, Labeled detection oligo: 5 nM (for fluorophore-labeled oligos), RNAsin: 1 μL/mL. Incubate at 37 C for 30 minutes, reaction volume 50 μL Wash twice with PBSTR.

### Example 2

### Highly Multiplexed Simultaneous Detection of RNAs and Proteins in Single Cells

Precise gene expression measurement has been fundamental to developing an advanced understanding of the roles of biological networks in health and disease. To enable detection of expression signatures specific to individual cells we developed PLAYR (Proximity Ligation Assay for RNA). PLAYR enables highly multiplexed quantification of transcripts in single cells by flow- and mass-cytometry and is compatible with standard antibody staining of proteins. This therefore enables simultaneous quantification of more than 40 different mRNAs and proteins. The technology was demonstrated in primary cells to be capable of quantifying multiple gene expression transcripts while the identity and the functional state of each analyzed cell was defined based on the expression of other transcripts or proteins. PLAYR now enables high throughput deep phenotyping of cells to readily expand beyond protein epitopes to include RNA expression, thereby opening a new venue on the characterization of cellular metabolism.

Biological systems operate through the functional interaction and coordination of multiple cell types. Whether one is trying to delineate the complexity of an immune response, or characterize the intrinsic cellular diversity of cancer, the ability to perform single-cell measurements of gene expression within such complex samples can lead to a better understanding of system-wide interactions and overall function.

A current method of choice for study of transcript expression in individual cells is single-cell RNA-seq. This approach involves physical separation of cells using FACS sorting or microfluidic-based devices, followed by lysis and library preparation with protocols that have been optimized for extremely small amounts of input RNA. Barcoding of physically separated cells before sequence analysis makes possible the analysis of thousands of individual cells in a single experiment. However, sample handling (such as physical separation of live cells before lysis and library preparation) has been shown to induce significant alterations in the transcriptome. Moreover RNA-seq requires cDNA synthesis and does not currently enable simultaneous detection of protein epitopes and transcripts. The complexity of protocols and the associated costs further limit the applicability of this technology in clinical settings and population studies, where sample throughput is essential. Finally, the number of cells that can be analyzed is limited by the overall sequencing depth available.

A complementary approach is to quantify a smaller number of transcripts while increasing the number of cells that can be analyzed. Flow cytometry allows multiple parameters to be measured in hundreds to thousands of cells per second. For such a purpose, for instance, fluorescence in situ hybridization (FISH) protocols have been adapted to quantify gene expression on cytometry platforms. In such experiments very bright FISH signals with excellent signal-to-noise ratios are necessary since flow cytometry does not provide the subcellular imaging resolution necessary to distinguish individual RNA signals from diffuse background. Different techniques have been adapted for the generation and amplification of specific hybridization signals including DNA padlock probes in combination with rolling circle amplification (RCA) or branched DNA technology. Recently the branched DNA approach has been successfully applied to flow cytometry, thus enabling the simultaneous detection of transcripts and proteins in intact cells. However, the current availability of only three non-interfering branched DNA amplification systems and the spectral overlap of fluorescent reporters limit multiplexing, which in turn limits studies of multiple transcripts and gene regulatory networks of complex cellular populations. Each of the latter techniques has their place and relevance. What was missing for higher parameter purposes was a technology that allowed full access to the parameterization enabled by mass cytometry and multiplexed ion beam imaging, but which also allowed for protein epitopes to be simultaneously measured.

The Proximity Ligation Assay for RNA (PLAYR) system as described here addresses these limitations by enabling routine analyses of thousands of cells per second by flow cytometric approaches and simultaneous detection of protein epitopes and multiple RNA targets. The method preserves the native state of cells in the first step of the protocol and detects transcripts in intact cells without the need for cDNA synthesis. PLAYR is compatible with flow cytometry, mass cytometry, and imaging systems. With mass cytometry especially, this enables the simultaneous quantitative acquisition of more than 40 cellular parameters of protein and/or RNA transcripts. Thus, PLAYR provides a unique and flexible capability to the growing list of technologies that merge 'omics datasets (transcript, protein, and signaling levels) in single cells. We expect that a tool such as PLAYR will allow for deeper insights into complex cell populations such as exist in immune infiltrates of cancer as well as measures of cancer cell proteins and gene expression profiles.

### Results

Overview of the Technology and PLAYR Probe Design.

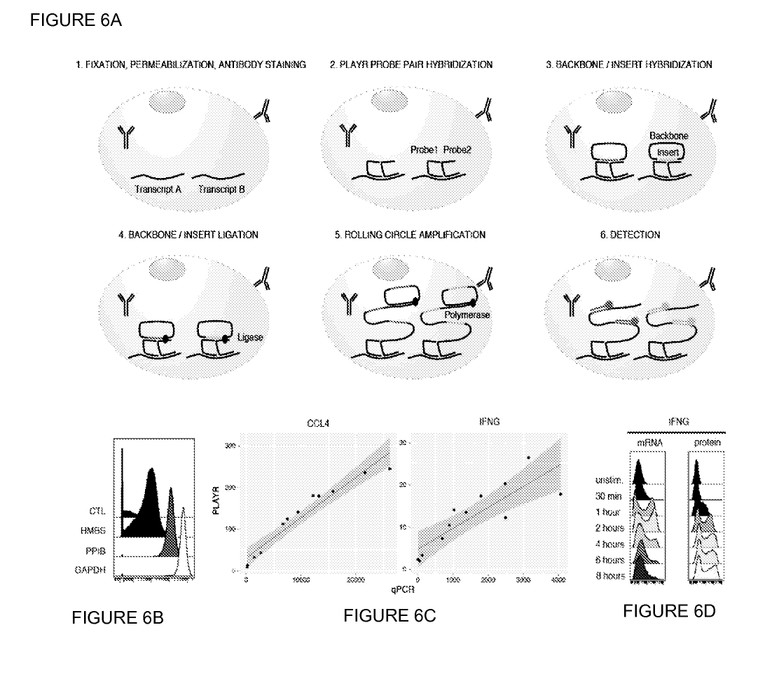

PLAYR uses the concept of proximity ligation to detect individual transcripts in single cells, as shown schematically in FIG. 6a, and is compatible with immunostaining. Pairs of DNA oligonucleotide probes (probe pairs) are designed to hybridize to two adjacent regions of target transcripts in fixed and permeabilized cells. Each probe in a pair is composed of two regions with distinct function. The role of the first region is to selectively hybridize to its cognate target RNA sequence. The second region, separated from the first by a short spacer, acts as template for the binding and circularization of two additional oligonucleotides (termed backbone and insert). When hybridized to two adjacent probes the backbone and insert oligonucleotides form a single-stranded DNA circle that can be ligated. The ligated, closed circle is then amplified through rolling circle amplification by phi29 polymerase initiated by the 3′ OH of one of the probes in a pair. As phi29 continues to polymerize, it creates a linear molecule that contains hundreds of concatenated complementary copies of the original circle. Then, using a labeled oligonucleotide that is complementary to the insert region of the amplicon, one can detect any given probe pair through binding to the amplified product. For analysis by flow cytometry fluorescently labeled oligonucleotides are used for detection. Alternatively, metal-conjugated oligonucleotides enable mass cytometric analyses using a CyTOF instrument.

Lowering of background binding events and increased specificity result from the fact that both PLAYR probes must hybridize independently to adjacent locations of a target RNA in order for the two independent ligation events and subsequent RCA to take place. Non-specific, off-target binding of single probes did not result in a signal (since single probes cannot serve as templates for backbone/insert circle formation). PLAYR can be multiplexed by designing oligonucleotides with different insert regions that act as cognate barcodes for given transcripts. Insert sequences are designed to have similar melting temperatures and base compositions to ensure they act as equally efficient templates for the formation of RCA products. To ensure that the resulting RCA products uniquely barcode a particular transcript the insert sequences do not have common substrings longer than 4 bases, as per our design specification software.

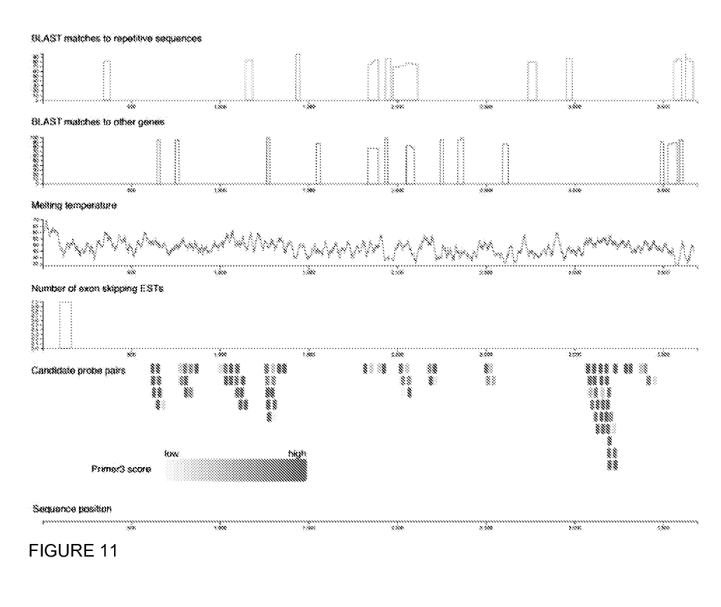

An open-source R software package with a GUI front end has been developed for rapid, user-friendly design of PLAYR probes (FIG. 11). Candidate probe pairs with similar thermodynamic properties are first produced using the Primer3 software. The application then displays the location of the probes along the target transcript sequence and other characteristics including BLAST matches to other transcripts or to repetitive sequences of the genome and the position of non-constitutively spliced exons. These features are used to guide the selection of specific probe pairs in a manner similar to that used in the OligoWiz microarray probe design software. For each gene, the user can then manually select the best probe pairs in combination with one of the PLAYR insert systems for multiplexing. Based on these selection criteria the software outputs the complete sequences of PLAYR probes that can be used to detect transcripts of interest. The sequences of all probes and backbone/insert systems used in this manuscript can be found in Table 1.

Simultaneous Quantification of Specific Proteins and Transcripts in Single Cells by Flow and Mass Cytometry.

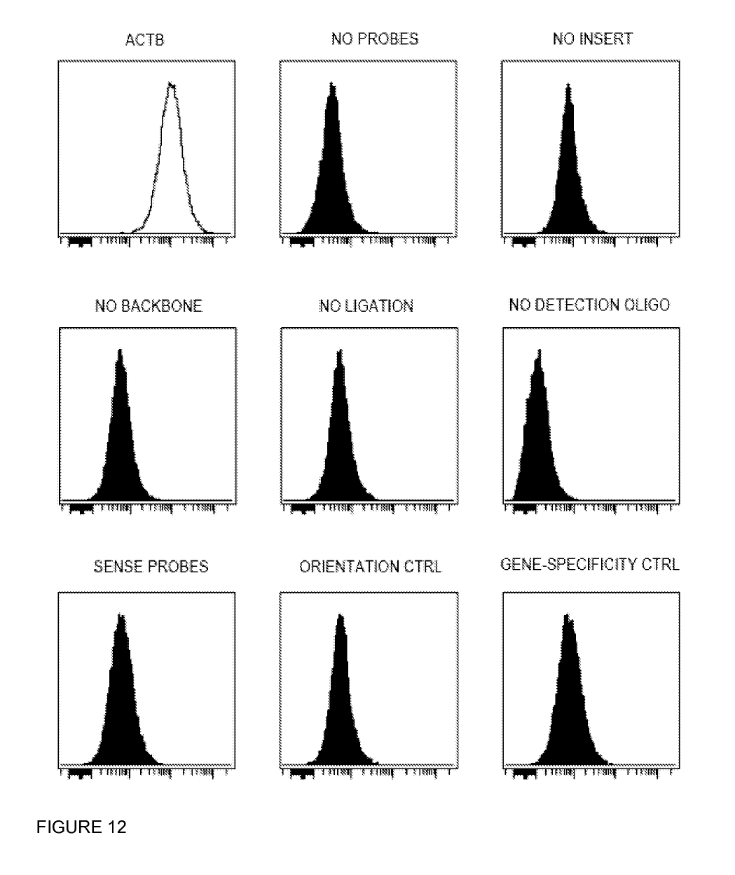

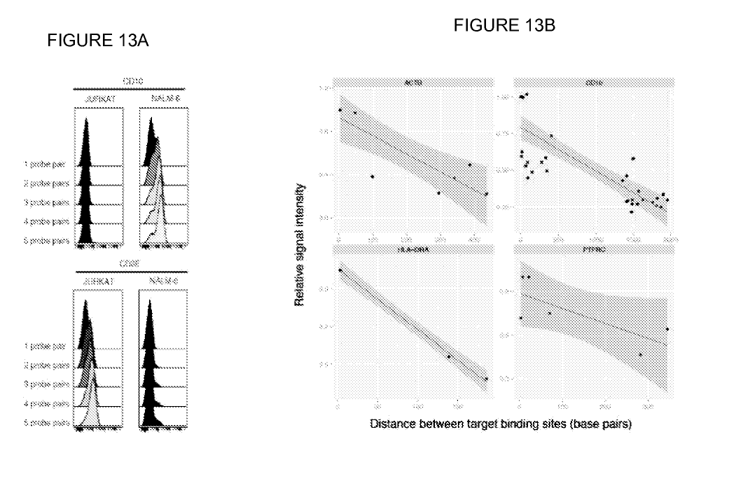

In a first experiment probe pairs specific for beta-actin (ACTB) were designed. In Jurkat T cells that had been fixed and permeabilized, the PLAYR protocol led to a signal that was detected well above background by flow cytometry (FIG. 12). No signal was observed if any component of the signal generation or amplification cascade was omitted. Similarly, no signal was observed when sense probes (i.e. identical to the transcript sequence instead of complementary), probes with the same half of the insert-targeted sequence, or combinations of probes targeting different genes were used (FIG. 12). To further demonstrate the specificity of the approach, the protocol was used with one or several probe pairs designed to detect CD10 and CD3E transcripts, which are known to be expressed in pre-B cells and T cells, respectively. CD10 mRNA was detected in NALM-6 cells, the pre-B cells, but not in Jurkat cells, a T cell line, whereas CD3E was detected in Jurkat cells but not NALM-6 cells (FIG. 13). As expected, signal intensities for these transcripts increased when multiple PLAYR probe pairs were used simultaneously. Interestingly, the resulting signal increase was in certain cases more than additive. This may be due to formation of RCA products generated from probes of two different pairs on the same transcript as might occur when the target regions of the two probes are not immediately adjacent. For instance, bound probes may be brought into proximity in unexpected manners by the structure of RNA molecules in three dimensions. Supporting this there was a strong, albeit not perfect, inverse relationship between the strength of the signal and the distance in the target hybridization regions of the two probes in a pair when multiple probe pairs were evaluated (FIG. 13). Thus, using more than one probe pair per transcript leads to an increase in signal and can also make results for individual genes more reproducible as it limits variability due to differences in probe accessibility to target sites.

In general we found that 4-5 probe pairs per gene led to reliable detection of both rare and highly abundant transcripts and we optimized the post-hybridization washes accordingly. We note that careful design of probe pairs and insert sequences could be used to delineate splice variant complexities and genomic translocations in genes of interest. Using 5 probe pairs per gene we detected the three housekeeping genes HMBS, PPIB, and GAPDH in U937 cells by mass cytometry (FIG. 6b). This application demonstrates a dynamic range of PLAYR that enables the detection of highly abundant transcripts (GAPDH) as well as low abundant transcripts (HMBS) that have been detected at only about 10 copies per U937 cell using other technologies. To further investigate to what extent PLAYR signals correlate with the underlying abundance of a transcript, results obtained with PLAYR and with RT-qPCR were compared for the induction of the cytokines interferon gamma (IFNG) and chemokine ligand 4 (CCL4) in the natural killer cell line NKL at different time points after stimulation with PMA/ionomycin. As shown in FIG. 6c, PLAYR and qPCR measurements were correlated (R-squared values of 0.93 (CCL4) and 0.72 (IFNG)), indicating that PLAYR reliably quantifies changes in transcript abundance across different biological conditions.

An additional important requirement in the optimization of the PLAYR protocol was that the approach should enable the simultaneous detection of transcripts and proteins. The protocol was therefore optimized using conditions that preserve binding of antibodies. Best results were obtained when antibody staining was performed immediately after cell fixation (i.e. at the beginning of the protocol). After antibody staining amine-to-amine crosslinking using the BS³ crosslinker was used to prevent antibodies from being washed away during the procedure. Critically, we found that transient permeabilization of cells by the addition of 0.2% saponin in the presence of RNase inhibitors during antibody staining greatly enhanced the preservation of RNA integrity. Furthermore, this transient permeabilization can be leveraged to stain intracellular proteins with antibodies. Using this protocol NKL cells were stimulated with PMA/ionomycin, in presence of protein-secretion inhibitors, and changes in IFNG protein and transcript levels were determined as a function of time (FIG. 1d). The IFNG mRNA was detected beginning at 30 minutes, and protein accumulation was first observed by 1 hour. Thus, PLAYR allows studies of the dynamic nature of transcription and translation at the single-cell level. Moreover, by monitoring gene expression directly, it is possible to detect early cell activation events, as transcription precedes translation.

Highly Multiplexed Detection of Specific Proteins and Transcripts in Single Cells by Mass Cytometry.

Using the insert-based multiplexing strategy illustrated in FIG. 1a, multiple targets can be detected simultaneously within populations of individual cells. We designed probes to target 14 different transcripts and first evaluated them individually and then together (simultaneously) in Jurkat T cells by mass cytometry (FIG. 2a). For this experiment cells were incubated either with probes against individual transcripts or with a mixture of all probes. Appropriate control combinations of non-cognate probe pairs were included to demonstrate probe pair specificity. Critically, the presence of insert/backbone oligonucleotides did not lead to observable signals if corresponding cognate probes were not also present in the reaction. Furthermore, the signal amplitude for any given target in the multiplexed sample was not affected by the presence of oligonucleotides against non-cognate targets and corresponding amplification products. This suggests that the number of transcripts that can be quantified within the same cell is only limited by the number of reporters that can be conjugated to detection oligonucleotides and analyzed simultaneously with a given platform.

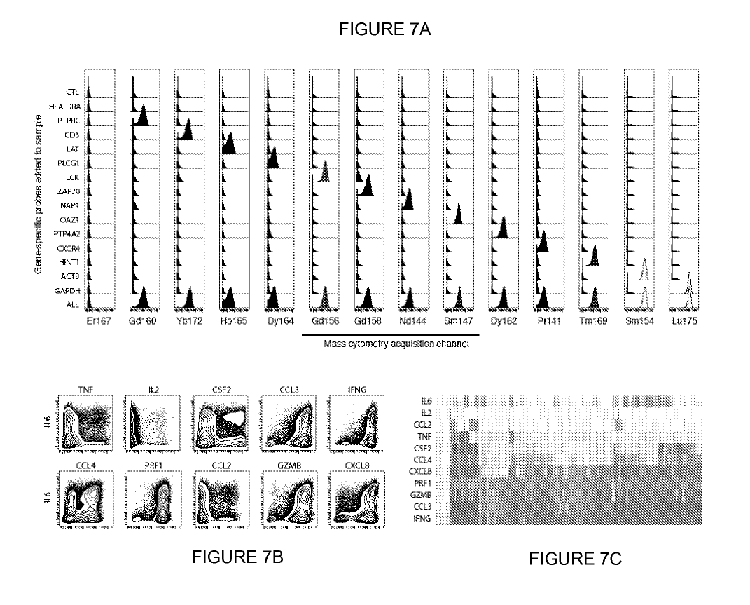

We made use of the multiplexing capability of PLAYR to simultaneously detect the transcripts of 11 different cytokines and other effector molecules in NKL cells that had been activated with three cytokines (IL2/IL12/IL18) and stimulated with PMA/ionomycin. Instead of a uniform cellular response, simultaneous transcript quantification revealed complex combinatorial RNA expression patterns in this supposedly homogenous clonal cell line (FIG. 7b). Based on such multiplexed measurements, high-dimensional analysis methods can be leveraged to identify functional NKL subpopulations based on transcript expression profiles. To that end we clustered cells based on the expression of induced effector transcripts, which revealed a remarkable complexity of cellular responses and distinct subpopulation of NKL cells that expressed defined combinations of effector molecules (FIG. 7c). A number of studies have shown that supposedly homogenous cell populations in primary samples also express such a remarkable diversity of cytokine combinations. While the functional implications of this observation are still poorly understood, the study of any such combinatorial phenomenon clearly benefits from the increased parameterization enabled by PLAYR.

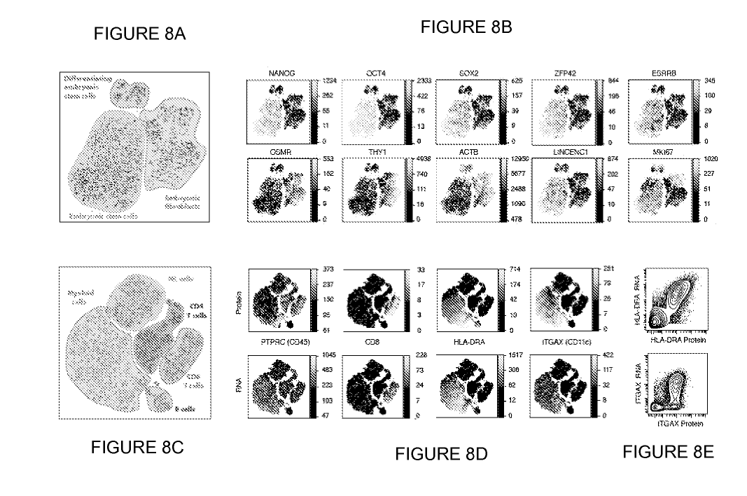

The increased multiplexing capabilities of PLAYR also enable RNA-only experiments, where transcript expression is used to define different cell types in which expression patterns of other transcripts can then be studied. Such experiments can be set up at a fraction of the costs typically associated with antibody-based experiments and are not limited by the availability of antibodies for genes of interest. We analyzed an artificial mixture of cells that contained mouse embryonic fibroblasts (MEFs), mouse embryonic stem cells (mESCs), and differentiating mESCs based on the expression of 15 different transcripts. We then visualized the data using viSNE, an algorithm that maps high-dimensional cytometry data onto two dimensions in a manner that best separates cell populations from the original high-dimensional space. This type of analysis clearly defined the three different populations of cells in the mixture based on RNA expression (FIG. 8a). Subsequently, different markers of pluripotency (e.g. NANOG), differentiation (e.g. THY1), proliferation (MKI67), as well as pluripotency-associated long intergenic non-coding RNAs (LINCENC1) could be studied in the context of this cellular system (FIG. 8b).

We further validated this approach by making use of the protein co-detection and multiplexing capabilities of PLAYR. For this experiment we analyzed primary human peripheral blood mononuclear cells (PBMCs) for 10 cell surface proteins and corresponding transcripts. In contrast to the previous experiment, antibody stained protein markers were used to create a viSNE³⁵ analysis. These protein epitope measurements enabled the visualization of the major cell types in human peripheral blood (FIG. 8c). Subsequent addition of the data on expression of corresponding transcripts demonstrated remarkable cell-type specificity in mRNA expression patterns (FIG. 8d). Moreover, this analysis revealed a discrepancy in the case of ITGAX, for which the protein but not the transcript was detected in a distinct subpopulation of cells (FIG. 8e). This demonstrates the potential of PLAYR to study the relationship of transcripts and proteins in subpopulations of cells within complex primary samples.

Profiling of Cytokine Transcript Induction in Complex Primary Samples.

We next used PLAYR to monitor cytokine transcript induction in PBMCs upon stimulation with lipopolysaccharide (LPS) to correlate protein marker expression with the functional capacity of individual cells. Cytokine expression in single cells is traditionally evaluated on the protein-level by flow cytometry after treatment with secretion inhibitors that lead to accumulation of cytokines in the cells. This approach precludes the study of (and is complicated by) paracrine effects, such as intercellular communication and feedback loops. We used antibodies against surface markers to distinguish different cell populations within human PBMCs while monitoring the expression of a panel of cytokine genes at the transcript level with PLAYR. Similar experiments were performed using fluorescence-based flow cytometry and mass cytometry. The fluorescence experiment involved the detection of four transcripts and four surface markers, whereas mass cytometry allowed for the simultaneous quantification of 8 transcripts and 18 protein epitopes including phosphorylation sites.

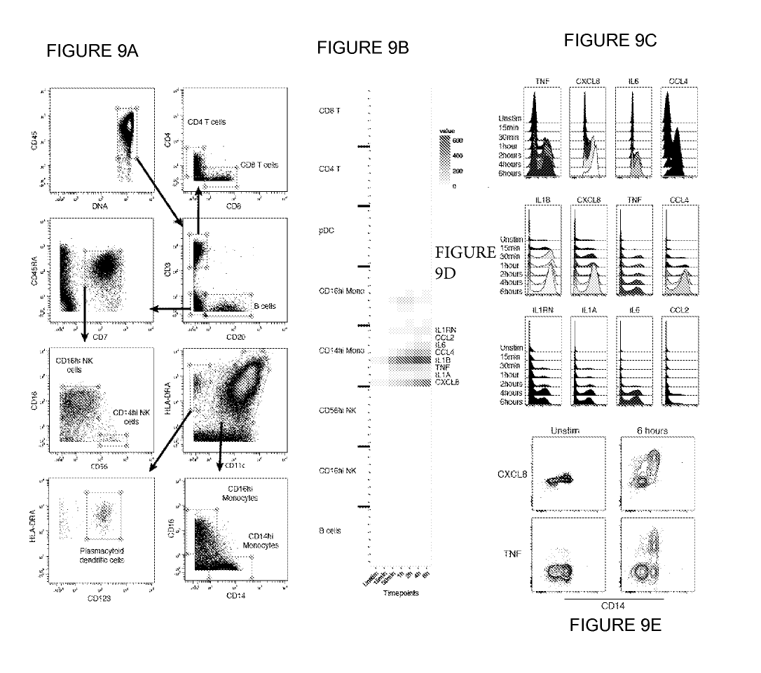

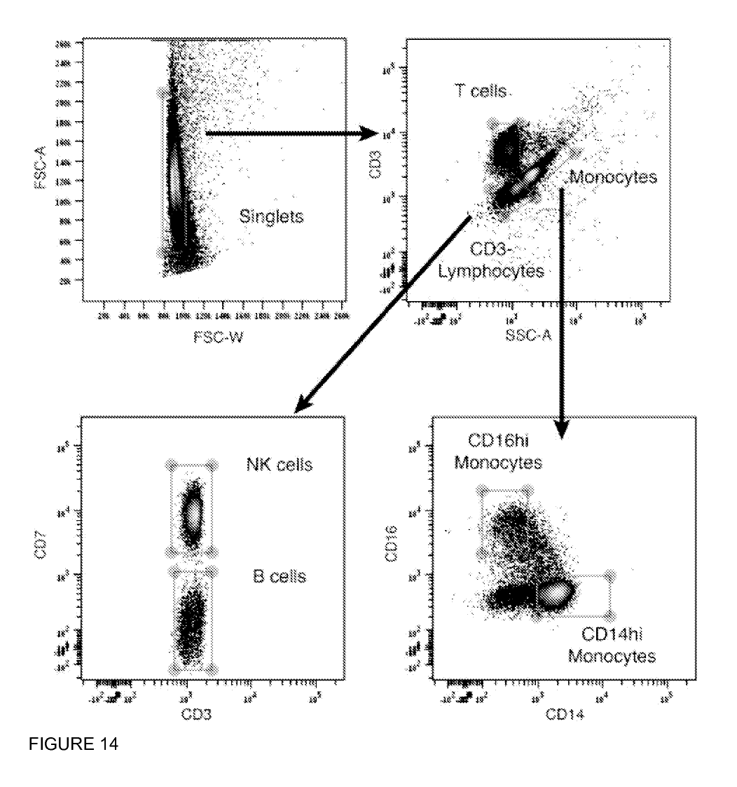

In both experiments, antibody staining enabled gating of different cell populations (gating for mass cytometry shown in FIG. 9a, see FIG. 14 for flow cytometry). As expected cytokine production was restricted to the CD33⁺ monocyte compartment and therein mostly to individual cells that expressed the LPS co-receptor CD14 (shown in heat map form in FIG. 4b). Moreover, different cytokines consistently exhibited distinct expression dynamics. For example, tumor necrosis factor alpha (TNF) and interleukin 8 (CXCL8) were induced early and the former peaked between 2 and 4 hours, while the latter continued to increase during the entire time course. Conversely, expression of interleukin 6 (IL6) was delayed and only strongly induced after 4 hours (FIGS. 9c and 9d). These results recapitulated previous individual observations and confirmed that PLAYR effectively detects RNA expression in specific cellular subpopulations. Interestingly, while CXCL8 at its peak is expressed in the entire CD14+ monocyte compartment, there was a distinct population of CD14+ cells that did not express TNF (FIG. 9e). This observation underscores the usefulness of protein and RNA co-detection in identifying functional differences in cellular populations.

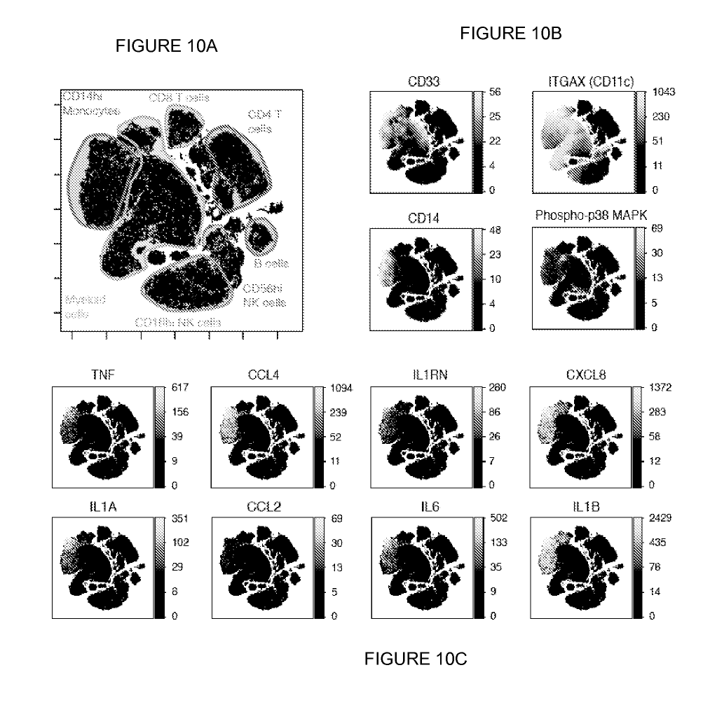

viSNE analysis using the CyTOF data for the cytokine induction experiment demonstrated that all major PBMC populations clustered in unique areas of the viSNE map (FIG. 10a) and could be identified by looking at the restricted expression of canonical markers (FIG. 10b). Similarly, MAP kinase signaling as measured by p38 MAP kinase phosphorylation could be monitored and was restricted to the myeloid compartment. When cytokine transcript expression was overlaid on the map, cells that responded to LPS were mostly restricted to the CD14+ monocytes region (FIG. 10c). This analysis provides a single-cell resolution map of cytokine induction and MAP kinase signaling in PBMCs, highlighting the potential of PLAYR in combination with mass cytometry for system-wide analyses of transcriptional networks in complex samples.

PLAYR enables highly multiplexed measurement of gene expression in hundreds to thousands of intact cells per second. On the protein level, single cell measurements have been shown to have prognostic and diagnostic value in multiple clinical settings. PLAYR extends such analyses to include measurements on the transcript level and could supplement the use of antibodies especially where exon-specific expression is concerned and no relevant antibody reagents exist. Immediate measurement of mRNA as enabled by PLAYR could overcome issues introduced with ex vivo processing of live cells in RNA-seq and related protocols. Experimental artifacts would also be further minimized since PLAYR assays for RNA molecules through direct binding and without the need for cDNA synthesis.

PLAYR can simultaneously measure transcripts and their encoded proteins, thus enabling the characterization of the interplay between transcription and translation at the single-cell level. Post-transcriptional and translational regulation of gene expression has been shown to be particularly important in several contexts, including early development, synaptic plasticity, inflammation and cancer, and PLAYR can be deployed to shed light on the underlying mechanisms with single-cell resolution. Other applications include clustering of complex cellular populations purely on the basis of transcript abundance, which is particularly useful when the availability or quality of antibodies is limiting. We believe that such an approach will help in the definition of cellular populations that share specific patterns of temporal or spatial regulation of RNA expression. Of relevance to this last point, PLAYR can be deployed for imaging approaches such as fluorescence microscopy and multiplexed ion beam imaging, making it a flexible tool to study gene expression in single intact cells on a variety of platforms.

### Methods

Tissue Culture.

Jurkat E6-1 (ATCC TIP-152), NALM-6 (DSMZ ACC128), and NKL (gift from Dr. Lewis Lanier, UCSF) cells were cultured in RPMI 1640 medium (Life Technologies) supplemented with 10% fetal bovine serum (Omega Scientific), 100 U/mL penicillin and 100 μg/mL streptomycin (Life Technologies), and 2 mM L-glutamine (Life Technologies) at 37° C. with 5% CO₂. For measurements of individual cytokine transcripts (FIG. 6), NKL cells were treated with 1×Protein Transport Inhibitor Cocktail (eBioscience) and 1×Cell Stimulation Cocktail (eBioscience). For combinatorial measurements of cytokine transcripts (FIG. 7), NKL cells were cultured as described above with the addition of 200 U/ml of rhlL-2 (NCI Biological Resources Branch), activated with 200 U/ml of rhlL-2, 10 ng/mL rhlL-12 (Peprotech), and 20 ng/mL rhlL-18 (R&D Systems) for 24 hours and treated with 150 ng/ml PMA (Sigma-Aldrich) plus 1 μM ionomycin (Sigma-Aldrich) for 3 hours in the presence of 1×Brefeldin A (eBioscience) and 1×Monensin (eBioscience). Mouse embryonic fibroblasts were prepared as described elsewhere and cultured in DMEM (Life Technologies), 10% fetal bovine serum, 2-mercaptoethanol (Sigma Aldrich), 1 mM sodium pyruvate (Life Technologies), 1×non-essential amino acids (Life Technologies), 100 U/mL penicillin and 100 μg/mL streptomycin. Mouse embryonic stem cells (ATCC CRL18-21) were grown on gelatin coated plates in DMEM, 10% fetal bovine serum, 2-mercaptoethanol, 1 mM sodium pyruvate, 1×non-essential amino acids, 100 U/mL penicillin, 100 μg/mL streptomycin, 1000 U/mL LIF (ESGRO, EMD Millipore), and 1×2i (MEK/GSK3 Inhibitor Supplement, EMD Millipore). Differentiation of embryonic stem cells was induced by withdrawal of 2i and LIF from the culture medium for two days. Human peripheral blood was purchased from the Stanford Blood Bank and was collected according to a Stanford University IRB-approved protocol. PBMCs were separated from whole blood using Ficoll (Thermo) and cryopreserved in liquid nitrogen. For analysis, PBMCs were thawed, washed with complete RPMI medium, and rested for 30 min at 37° C. under 5% CO₂ in complete RPMI medium. PBMCs were stimulated with LPS (InvivoGen) at a concentration of 10 ng/mL in complete RPMI medium under gentle agitation.

Cell fixation, permeabilization, and antibody staining. Cells at a density of ˜1×10⁶/mL were fixed in RPMI medium without serum in 1.6% paraformaldehyde (Electronic Microscopy Sciences) for 10 min at room temperature under gentle agitation as described previously⁴⁸. For detection of protein epitopes, cells were stained with antibodies in PBS (Life Technologies) supplemented with 5 mg/mL UltraPure BSA (Life Technologies), 0.2% saponin (Sigma-Aldrich), 2.5% v/v polyvinylsulfonic acid (Polysciences), and 40 U/mL RNasin (Promega) for 30 min at room temperature. After washing, antibodies were crosslinked to the cells with 5 mM bis(sulfosuccinimidyl) suberate (Pierce) in a buffer containing PBS, 0.2% saponin, and 40 U/mL RNasin for 30 min at room temperature at a density of ˜20×10⁶ cells/mL. Glycine was added to a final concentration of 100 mM, and samples were incubated for 5 min. Cells were pelleted and permeabilized with ice-cold methanol for at least 10 min on ice. Once in methanol cells can be stored at −80° C. for several weeks without loss of antibody signal or RNA degradation. For detection of RNA only, cells were permeabilized in ice-cold methanol immediately after fixation with paraformaldehyde. Antibodies used for mass cytometry: CD19 (HIB19), CD38 (HIT2), CD4 (RPA-T4), CD8 (RPA-T8), CD7 (CD7-6B7), CD14 (RM052), CD123 (6H6), CD45 (HI30), CD45RA (HI100), CD33 (WM53), CD11c (Bu15), CD16 (3G8), CD3 (UCHT1), CD20 (2H7), HLA-DRA (L243), CD56 (NCAM 16.2) and phosphorylation sites pP38 MAPK (pT180/pY182), pERK1/2 (pT202/pY204). Antibodies used for flow cytometry: CD3 (UCHT1, Bv510), CD7 (M-T701, Alexa700), CD16 (3G8, Bv605), CD14 (HCD14, Bv421), BrdU (Bu20a, PE), Biotin (Streptavidin, PE-Cy7).

PLAYR Protocol.

PLAYR probes were designed using the PLAYRDesign software developed in-house. PLAYR probes were synthesized at the Stanford Protein and Nucleic Acid Facility and resuspended in DEPC-treated water at a concentration of 100 μM. The carrier solution for most of the protocol steps, including washes, was PBS, 0.1% Tween (Sigma-Aldrich), and 4 U/mL RNasin. Paraformaldehyde-fixed and methanol-permeabilized cells (see above) were pelleted by centrifugation at 600 g for 3 min. Hybridizations with PLAYR probes were performed in a buffer based on DEPC-treated water (Life Technologies) containing 1×SSC (Affymetrix), 2.5% v/v polyvinylsulfonic acid, 20 mM ribonucleoside vanadyl complex (New England Biolabs), 40 U/mL RNasin, 1% Tween, and 100 μg/mL salmon sperm DNA (Life Technologies). PLAYR probes for all target transcripts of an experiment were mixed and heated to 90° C. for 5 min. Probes were then chilled on ice and added to cells in hybridization buffer at a final concentration of 100 nM. Cells were incubated for 1 h at 40° C. under vigorous agitation, and subsequently washed three times. Cells were then incubated for 20 min in a buffer containing PBS, 4×SSC, 40 U/mL RNasin at 40° C. under vigorous agitation. Samples to be analyzed by mass cytometry were barcoded at this step as described previously. After two washes, cells were incubated with 100 nM insert/backbone oligonucleotides in PBS, 1×SSC, 40 U/mL RNasin for 30 min at 37° C. After two washes, cells were incubated for 30 min with T4 DNA ligase (Thermo) at room temperature with gentle agitation, followed by a 2 h (flow cytometry) or 6 h (mass cytometry) incubation with phi29 DNA polymerase (Thermo) at 30° C. under agitation. Longer amplification (up to 16 h) generally increases signal intensity. Both enzymes were used according to manufacturers' instructions, with the addition of 40 U/mL RNasin. For flow cytometry, cells were incubated with detection oligonucleotides at a concentration of 5 nM for 30 minutes at 37° C. in PBS, 1×SSC, 0.1% Tween, 40 U/mL RNasin. Two fluorophore-conjugated (Alexa488 and Alexa647) oligonucleotides were used as detection probes. Also used were a biotinylated oligonucleotide and an oligonucleotide labeled with a single BrdU nucleotide at the 5′ end; cells were then incubated with PE-Cy7-streptavidin or an anti-BrdU-PE antibody conjugate as appropriate. For mass cytometry, cells were incubated with metal-conjugated detection oligonucleotides at a concentration of 10 nM for 30 minutes at 37° C. in PBS, 5 mg/mL BSA, 0.02% sodium azide. After washing, cells were processed immediately on a fluorescence-based flow cytometer or further processed for CyTOF acquisition as described elsewhere.

Preparation of Metal-Conjugated Detection Oligonucleotides.

Maleimide-activated Maxpar metal chelating X8 polymers (Fluidigm, Maxpar labeling kit) were loaded with metals and purified using centrifugal filters as per the manufacturer's instructions. Detection oligonucleotides carrying a 5′ Thiol-Modifier C6 S-S(Glen Research) were synthesized at the Stanford Protein and Nucleic Acid Facility. Oligonucleotides were resuspended in DEPC-treated water at 250 μM, and the thiol was reduced by treatment with 50 mM TCEP (Pierce) for 30 min at room temperature. After ethanol precipitation, oligonucleotides were resuspended in C buffer (Fluidigm, Maxpar labeling kit) and conjugation reactions were performed with 2 nmol of oligonucleotide per reaction with X8 polymer. After 2 h at room temperature, TCEP was added to a final concentration of 5 mM, and samples were incubated for 30 min to reduce unconjugated oligonucleotides. Conjugates were filtered through 30-kDa centrifugal filter units (EMD Millipore) in a total of 500 μl water, spun at 14000 g for 12 min, and washed twice with DEPC-treated water (Life Technologies). Purified detection oligonucleotide conjugates were resuspended in DEPC-treated water at a concentration of 1 μM and stored at 4° C.

RT-qPCR.

RNA was extracted using RNeasy Plus Micro Kit (Qiagen), following the manufacturer's instructions. Reverse transcription was performed using SuperScript III First-Strand Synthesis System (Life Technologies), following the manufacturer's instructions. PCR was carried out in a LightCycler 480 II (Roche) using SYBRGreen I Master (Roche).

TABLE 1
Primer sequences

PLAYR1 Insert 1
AAAAAAAAAACTCAGTCGTGACACTCTT
PLAYR1 Insert 4
AAAAAAAAAACTACCTTGGGACACTCTT
PLAYR1 Insert 7
AAAAAAAAAACCGCTTATGGACACTCTT
PLAYR1 Insert 8
AAAAAAAAAACTCGATCTGGACACTCTT
PLAYR1 Insert 11
AAAAAAAAAATGACTCTCGGACACTCTT
PLAYR1 Insert 13
AAAAAAAAAATTCTCCAGGGACACTCTT
PLAYR1 Insert 15
AAAAAAAAAACTTCTGCAGGACACTCTT
PLAYR1 Insert 16
AAAAAAAAAATCTATCCGGGACACTCTT
PLAYR1 Insert 17
AAAAAAAAAACGCATCTTGGACACTCTT
PLAYR1 Insert 19
AAAAAAAAAATCGCTACTGGACACTCTT
PLAYR1 Insert 20
AAAAAAAAAATACGCTCTGGACACTCTT
PLAYR1 Insert 22
AAAAAAAAAACCATTCGTGGACACTCTT
PLAYR1 Insert 25
AAAAAAAAAATTCGCACTGGACACTCTT
PLAYR1 Insert 26
AAAAAAAAAATCCTTCAGGGACACTCTT
PLAYR2 Insert 1
AAAAAAAAAAGACGCTAATATCGTGACC
PLAYR2 Insert 4
AAAAAAAAAAGACGCTAATCAGGCTACT
PLAYR2 Insert 7
AAAAAAAAAAGACGCTAATCTACATGGC
PLAYR2 Insert 8
AAAAAAAAAAGACGCTAATCAACCTGGT
PLAYR2 Insert 11
AAAAAAAAAAGACGCTAATCTCGGAATC
PLAYR2 Insert 13
AAAAAAAAAAGACGCTAATCTCAATCGG
PLAYR2 Insert 15
AAAAAAAAAAGACGCTAATCCAGGATCT
PLAYR2 Insert 16
AAAAAAAAAAGACGCTAATCTGTAGACC
PLAYR2 Insert 17
AAAAAAAAAAGACGCTAATCTGGCACAT
PLAYR2 Insert 19
AAAAAAAAAAGACGCTAATCGCCATGAT
PLAYR2 Insert 20
AAAAAAAAAAGACGCTAATCACACTTGG
PLAYR2 Insert 22
AAAAAAAAAAGACGCTAATCATCAGCGT
PLAYR2 Insert 25
AAAAAAAAAAGACGCTAATCAATTCCGG
PLAYR2 Insert 26
AAAAAAAAAAGACGCTAATCCGCTAAGT
BACKBONE AND INSERT SEQUENCES
BACKBONE
P-ATTAGCGTCCAGTGAATGCGAGTCCGTCTAGGAGAGTAGTACAGCAGC
CGTCAAGAGTGTC
Insert 1
P-ACGACTGAGTTTGGTCACGAT
Insert 4
P-CCAAGGTAGTTTAGTAGCCTG
Insert 7
P-CATAAGCGGTTTGCCATGTAG
Insert 8
P-CAGATCGAGTTTACCAGGTTG
Insert 11
P-CGAGAGTCATTTGATTCCGAG
Insert 13
P-CCTGGAGAATTTCCGATTGAG
Insert 15
P-CTGCAGAAGTTTAGATCCTGG
Insert 16
P-CCGGATAGATTTGGTCTACAG
Insert 17
P-CAAGATGCGTTTATGTGCCAG
Insert 19
P-CAGTAGCGATTTATCATGGCG
Insert 20
P-CAGAGCGTATTTCCAAGTGTG
Insert 22
P-CACGAATGGTTTACGCTGATG
Insert 25
P-CAGTGCGAATTTCCGGAATTG
Insert 26
P-CCTGAAGGATTTACTTAGCGG
DETECTION SEQUENCES
Backbone Detection
ZCAGTGAATGCGAGTCCGTCT
Detection 1
ZACGACTGAGTTTGGTCACGAT
Detection 4
ZCCAAGGTAGTTTAGTAGCCTG
Detection 7
ZCATAAGCGGTTTGCCATGTAG
Detection 8
ZCAGATCGAGTTTACCAGGTTG
Detection 11
ZCGAGAGTCATTTGATTCCGAG
Detection 13
ZCCTGGAGAATTTCCGATTGAG
Detection 15
ZCTGCAGAAGTTTAGATCCTGG
Detection 16
ZCCGGATAGATTTGGTCTACAG
Detection 17
ZCAAGATGCGTTTATGTGCCAG
Detection 19
ZCAGTAGCGATTTATCATGGCG
Detection 20
ZCAGAGCGTATTTCCAAGTGTG
Detection 22
ZCACGAATGGTTTACGCTGATG
Detection 25
ZCAGTGCGAATTTCCGGAATTG
Detection 26
ZCCTGAAGGATTTACTTAGCGG
HMBS_1481_INS1
TTCAAGCTCCTTGGTAAACAGGCTAAAAAAAAAAGACGCTAATATCGTGA
CC
HMBS_1482_INS1
GTCCACTTCATTCTTCTCCAGGGCAAAAAAAAAACTCAGTCGTGACACTC
TT
HMBS_1483_INS1
TGGGTGAAAGACAACAGCATCATGAAAAAAAAAAAGACGCTAATATCGTG
ACC
HMBS_1484_INS1
TCTGGCAGGGTTTCTAGGGTCTTCAAAAAAAAAACTCAGTCGTGACACTC
TT
HMBS_1485_INS1
GAACTCCAGATGCGGGAACTTTCTAAAAAAAAAAGACGCTAATATCGTGA
CC
HMBS_1486_INS1
GGTGTTGAGGTTTCCCCGAATACTAAAAAAAAAACTCAGTCGTGACACTC
TT
HMBS_1487_INS1
CTACCAACTGTGGGTCATCCTCAGAAAAAAAAAAGACGCTAATATCGTGA
CC
HMBS_1488_INS1
TCGTGGAATGTTACGAGCAGTGATAAAAAAAAAACTCAGTCGTGACACTC
TT
HMBS_1489_INS1
CAGATAGCAGTGAGAATGGGGCACAAAAAAAAAAGACGCTAATATCGTGA
CC
HMBS_1490_INS1
TTCAGTCTCCCGGGGTAATCACTCAAAAAAAAAACTCAGTCGTGACACTC
TT
PPIB_1491_INS13
GATCACCCGGCCTACATCTTCATCAAAAAAAAAAGACGCTAATCTCAATC
GG
PPIB_1492_INS13
GGAACAGTCTTTCCGAAGAGACCAAAAAAAAAAATTCTCCAGGGACACTC
TT
PPIB_1493_INS13
GCTCACCGTAGATGCTCTTTCCTCAAAAAAAAAAGACGCTAATCTCAATC
GG
PPIB_1494_INS13
TCAGTTTGAAGTTCTCATCGGGGAAAAAAAAAAATTCTCCAGGGACACTC
TT
PPIB_1495_INS13
GTGATGAAGAACTGGGAGCCGTTGAAAAAAAAAAGACGCTAATCTCAATC
GG
PPIB_1496_INS13
CATCTAGCCAGGCTGTCTTGACTGAAAAAAAAAATTCTCCAGGGACACTC
TT
PPIB_1497_INS13
AAAGGGCTTCTCCACCTCGATCTTAAAAAAAAAAGACGCTAATCTCAATC
GG
PPIB_1498_INS13
GAAAGATGTCCCTGTGCCCTACTCAAAAAAAAAATTCTCCAGGGACACTC
TT
PPIB_1499_INS13
CAAAAGTGAGTCCATGGGCCTGTGAAAAAAAAAAGACGCTAATCTCAATC
GG
PPIB_1500_INS13
TGGTCAGTGTTGGTAGGAGTTTGTAAAAAAAAAATTCTCCAGGGACACTC
TT
GAPDH_1501_INS4
TGGTTCACACCCATGACGAACATAAAAAAAAAAGACGCTAATCAGGCTAC
T
GAPDH_1502_INS4
TGCTGATGATCTTGAGGCTGTTGTAAAAAAAAAACTACCTTGGGACACTC
TT
GAPDH_1503_INS4
GACTGTGGTCATGAGTCCTTCCACAAAAAAAAAAGACGCTAATCAGGCTA
CT
GAPDH_1504_INS4
CAGTCTTCTGGGTGGCAGTGATGAAAAAAAAAACTACCTTGGGACACTCT
T
GAPDH_1505_INS4
CAGGTTTTTCTAGACGGCAGGTCAAAAAAAAAAAGACGCTAATCAGGCTA
CT
GAPDH_1506_INS4
CCTGCTTCACCACCTTCTTGATGTAAAAAAAAAACTACCTTGGGACACTC
TT
GAPDH_1507_INS4
GTCCAGGGGTCTTACTCCTTGGAGAAAAAAAAAAGACGCTAATCAGGCTA
CT
GAPDH_1508_INS4
TCTCTTCCTCTTGTGCTCTTGCTGAAAAAAAAAACTACCTTGGGACACTC
TT
GAPDH_1509_INS4
TGTGAGGAGGGGAGATTCAGTGTGAAAAAAAAAAGACGCTAATCAGGCTA
CT
GAPDH_1510_INS4
CCTCTTCAAGGGGTCTACATGGCAAAAAAAAAAACTACCTTGGGACACTC
TT
IFNG_281_INS11
TCGACCTCGAAACAGCATCTGAAAAAAAAAAATGACTCTCGGACACTCTT
IFNG_282_INS11
CAGGACAACCATTACTGGGATGCTAAAAAAAAAAGACGCTAATCTCGGAA
TC
IFNG_283_INS11
TCAAACCGGCAGTAACTGGATAGAAAAAAAAAATGACTCTCGGACACTCT
T
IFNG_284_INS11
AAGCACTGGCTCAGATTGCAGGCATAAAAAAAAAAGACGCTAATCTCGGA
ATC
IFNG_285_INS11
AGAACCCAAAACGATGCAGAGCTAAAAAAAAAATGACTCTCGGACACTCT
T
IFNG_286_INS11
ATATGGGTCCTGGCAGTAACAGCCAAAAAAAAAAGACGCTAATCTCGGAA
TC
IFNG_287_INS11
TGGAAGCACCAGGCATGAAATCTCAAAAAAAAAATGACTCTCGGACACTC
TT
IFNG_288_INS11
GGGTACAGTCACAGTTGTCAACAATAAAAAAAAAAGACGCTAATCTCGGA
ATC
CCL4_2101_INS22
CAGTCACGCAGAGCTTCATGGTATAAAAAAAAAAGACGCTAATCATCAGC
GT
CCL4_2102_INS22
AGAAGGCAGCTACTAGCATGAGGAAAAAAAAAAACCATTCGTGGACACTC
TT
CCL4_2103_INS22
TCAGGTGACCTTCCCTGAAGACTTAAAAAAAAAAGACGCTAATCATCAGC
GT
CCL4_2104_INS22
ATGTGTCTCATGGAGAAGCATCCGAAAAAAAAAACCATTCGTGGACACTC
TT
CCL4_2105_INS22
CCATAGGGGACACTTATCCTTTGGCAAAAAAAAAAGACGCTAATCATCAG
CGT
CCL4_2106_INS22
ACAGCAGAGAAACAGTGACAGTGGAAAAAAAAAACCATTCGTGGACACTC
TT
Primers used in FIG. 7
CD3E_1005_INS1
CCACTTTGCTCCAATTCTGAAAAAAAAAAAGACGCTAATATCGTGACC
CD3E_1006_INS1
TCCTCTGGGGTAGCAGACATAAAAAAAAAACTCAGTCGTGACACTCTT
CD3E_1007_INS1
GTAAACCAGCAGCAGCAAGCAAAAAAAAAAGACGCTAATATCGTGACC
CD3E_1008_INS1
CCTTGGCCTTTCTATTCTTGCAAAAAAAAAACTCAGTCGTGACACTCTT
CD3E_1009_INS1
TGGTGGCCTCTCCTTGTTTTAAAAAAAAAAGACGCTAATATCGTGACC
CD3E_1010_INS1
CTCATAGTCTGGGTTGGGAACAAAAAAAAAACTCAGTCGTGACACTCTT
CD3E_1011_INS1
CGTCTCTGATTCAGGCCAGAAAAAAAAAAAGACGCTAATATCGTGACC
CD3E_1012_INS1
CAGTGTTCTCCAGAGGGTCAGAAAAAAAAAACTCAGTCGTGACACTCTT
PTPRC_1017_INS7
TTTTGCAATGATGTAGGCATGAAAAAAAAAAGACGCTAATCTACATGGC
PTPRC_1018_INS7
GCAGCACTTCCATTACGTTGAAAAAAAAAACCGCTTATGGACACTCTT
PTPRC_1021_INS7
TTCCAACAAAATATCTGCATGGAAAAAAAAAAGACGCTAATCTACATGGC
PTPRC_1022_INS7
CCTTCATCAGCAATCTTCCTCAAAAAAAAAACCGCTTATGGACACTCTT
PTPRC_1023_INS7
GAAACTTGCTGAACACCCGAAAAAAAAAAGACGCTAATCTACATGGC
PTPRC_1024_INS7
TAAAGGGCTTTCGAGCTTCCAAAAAAAAAACCGCTTATGGACACTCTT
PTPRC_1025_INS7
CAGTTTGAGGAGCAAGTGAGGAAAAAAAAAAGACGCTAATCTACATGGC
PTPRC_1026_INS7
GCTGAAGGCATTCACTCTCCAAAAAAAAAACCGCTTATGGACACTCTT
GAPDH_1033_INS4
GTTAAAAGCAGCCCTGGTGAAAAAAAAAAAGACGCTAATCAGGCTACT
GAPDH_1034_INS4
TGATGGCAACAATATCCACTTTAAAAAAAAAACTACCTTGGGACACTCTT
GAPDH_1035_INS4
ATTGATGACAAGCTTCCCGTAAAAAAAAAAGACGCTAATCAGGCTACT
GAPDH_1036_INS4
TGGAAGATGGTGATGGGATTAAAAAAAAAACTACCTTGGGACACTCTT
GAPDH_1037_INS4
CATCGCCCCACTTGATTTTAAAAAAAAAAGACGCTAATCAGGCTACT
GAPDH_1038_INS4
TGGACTCCACGACGTACTCAAAAAAAAAAACTACCTTGGGACACTCTT
GAPDH_1039_INS4
TCATCATATTTGGCAGGTTTTTAAAAAAAAAAGACGCTAATCAGGCTACT
GAPDH_1040_INS4
CCTGCTTCACCACCTTCTTGAAAAAAAAAACTACCTTGGGACACTCTT
ACTB_1057_INS8
GTCAGGCAGCTCGTAGCTCTAAAAAAAAAAGACGCTAATCAACCTGGT
ACTB_1058_INS8
TGCCAATGGTGATGACCTGAAAAAAAAAACTCGATCTGGACACTCTT
ACTB_1059_INS8
ATGTCCACGTCACACTTCATGAAAAAAAAAAGACGCTAATCAACCTGGT
ACTB_1060_INS8
TGTTGGCGTACAGGTCTTTGAAAAAAAAAACTCGATCTGGACACTCTT
ACTB_1063_INS8
ATCTGCTGGAAGGTGGACAGAAAAAAAAAAGACGCTAATCAACCTGGT
ACTB_1064_INS8
CGTCATACTCCTGCTTGCTGAAAAAAAAAACTCGATCTGGACACTCTT
ACTB_1065_INS8
TCAAGAAAGGGTGTAACGCAAAAAAAAAAAGACGCTAATCAACCTGGT
ACTB_1066_INS8
TGTTTTCTGCGCAAGTTAGGTAAAAAAAAAACTCGATCTGGACACTCTT
HLA-DRA_1141_INS20
CAGATAGAACTCGGCCTGGAAAAAAAAAAAGACGCTAATCACACTTGG
HLA-DRA_1142_INS20
TAAACTCGCCTGATTGGTCAAAAAAAAAAATACGCTCTGGACACTCTT
HLA-DRA_1143_INS20
TTGTCCACAGCTATGTTGGCAAAAAAAAAAGACGCTAATCACACTTGG
HLA-DRA_1144_INS20
CGCTTTGTCATGATTTCCAGAAAAAAAAAATACGCTCTGGACACTCTT
HLA-DRA_1145_INS20
GTGACATTGACCACTGGTGGAAAAAAAAAAGACGCTAATCACACTTGG
HLA-DRA_1146_INS20
CAGGTTTTCCATTTCGAAGCAAAAAAAAAATACGCTCTGGACACTCTT
HLA-DRA_1151_INS20
CGTTCTGCTGCATTGCTTTAAAAAAAAAAGACGCTAATCACACTTGG
HLA-DRA_1152_INS20
CTCCATGTGCCTTACAGAGGAAAAAAAAAATACGCTCTGGACACTCTT
LCK_1203_INS16
ACCACAGCGTAGAGCCGAAAAAAAAAAAGACGCTAATCTGTAGACC
LCK_1204_INS16
GATGATGTAGATGGGCTCCTGAAAAAAAAAATCTATCCGGGACACTCTT
LCK_1205_INS16
CTCTTCAATGAATGCCATGCAAAAAAAAAAGACGCTAATCTGTAGACC
LCK_1206_INS16
CCCGAAGGTCACGATGAATAAAAAAAAAAATCTATCCGGGACACTCTT
LCK_1207_INS16
CAATGAGGCGTGCTAGGCAAAAAAAAAAGACGCTAATCTGTAGACC
LCK_1208_INS16
CTCCCTGGCTGTGTACTCGTAAAAAAAAAATCTATCCGGGACACTCTT
LCK_1209_INS16
ACCTCCGGGTTGGTCATCAAAAAAAAAAGACGCTAATCTGTAGACC
LCK_1210_INS16
GTAGCCTCGCTCCAGGTTCTAAAAAAAAAATCTATCCGGGACACTCTT
ZAP70_1211_INS17
AGTGGTACACCGTCTTCCCAAAAAAAAAAAGACGCTAATCTGGCACAT
ZAP70_1212_INS17
GCCTTGTCTTGGCTGATGAAAAAAAAAAACGCATCTTGGACACTCTT
ZAP70_1213_INS17
TATGAGGAGGTTATCGCGCTAAAAAAAAAAGACGCTAATCTGGCACAT
ZAP70_1214_INS17
CGCAGCCAAGTTCAATGTCAAAAAAAAAACGCATCTTGGACACTCTT
ZAP70_1215_INS17
CTCCAGGTACTTCATCCCCAAAAAAAAAAAGACGCTAATCTGGCACAT
ZAP70_1216_INS17
AGGTCACGGTGCACAAAGTTAAAAAAAAAACGCATCTTGGACACTCTT
ZAP70_1217_INS17
CCATAGCTCCAGACATCGCTAAAAAAAAAAGACGCTAATCTGGCACAT
ZAP70_1218_INS17
AGGACAAGGCCTCCCACATAAAAAAAAAACGCATCTTGGACACTCTT
LAT_1221_INS19
ACACAGTGCCATCAACATGGAAAAAAAAAAGACGCTAATCGCCATGAT
LAT_1222_INS19
CTGGCAGTCTGTGGCAGTGAAAAAAAAAATCGCTACTGGACACTCTT
LAT_1225_INS19
CTCATCCGCATCCTCACAGAAAAAAAAAAGACGCTAATCGCCATGAT
LAT_1226_INS19
GCCTGGGTTGTGATAGTCGTAAAAAAAAAATCGCTACTGGACACTCTT
LAT_1227_INS19
GCTGAGTGCAGGAGCTGATAAAAAAAAAAGACGCTAATCGCCATGAT
LAT_1228_INS19
AGAAGGCACTGTCTCGGATGAAAAAAAAAATCGCTACTGGACACTCTT
LAT_1229_INS19
CAGTTCCTGGGACACATTCAAAAAAAAAAAGACGCTAATCGCCATGAT
LAT_1230_INS19
CTCAGTCTTAGCCGCTCCAGAAAAAAAAAATCGCTACTGGACACTCTT
PLCG1_1241_INS22
TGGAGAACAGGAAGGTGACAAAAAAAAAAAGACGCTAATCATCAGCGT
PLCG1_1242_INS22
TGCGAGTTCCACACACTGTTAAAAAAAAAACCATTCGTGGACACTCTT
PLCG1_1243_INS22
TCTTGGTGGTAAGGGTGTGCAAAAAAAAAAGACGCTAATCATCAGCGT
PLCG1_1244_INS22
ATGGTGTGCAGGACATCTGAAAAAAAAAAACCATTCGTGGACACTCTT
PLCG1_1245_INS22
AGTGGTCCTCAATGGACAGGAAAAAAAAAAGACGCTAATCATCAGCGT
PLCG1_1246_INS22
ATGTTTCTCTGCTGGGCAATAAAAAAAAAACCATTCGTGGACACTCTT
PLCG1_1247_1N522
CCTCAGCCAGCTTCTTGTGAAAAAAAAAAGACGCTAATCATCAGCGT
PLCG1_1248_INS22
GTAGGCACCTCCTCGTAGGCAAAAAAAAAACCATTCGTGGACACTCTT
PTP4A2_1403_INS11
GCACAGGCTAATGTTCTGCTGAAAAAAAAAAGACGCTAATCTCGGAATC
PTP4A2_1404_INS11
GAGTGTGCTCCCCAAACAGAAAAAAAAAAATGACTCTCGGACACTCTT
PTP4A2_1405_INS11
CGCACATCTACAGCAACAAGGAAAAAAAAAAGACGCTAATCTCGGAATC
PTP4A2_1406_INS11
CTTAAGGCTGCCAGACTGCAAAAAAAAAAATGACTCTCGGACACTCTT
PTP4A2_1407_INS11
CCTGTTATGGCCTGTGGACAAAAAAAAAAAGACGCTAATCTCGGAATC
PTP4A2_1408_INS11
TGGGTTGCACGATGTCTCTCAAAAAAAAAATGACTCTCGGACACTCTT
PTP4A2_1411_INS11
CAGTGTGAGACAAGGTCCCGAAAAAAAAAAGACGCTAATCTCGGAATC
PTP4A2_1412_INS11
GCCGGTCAACATGTGAGGTAAAAAAAAAAATGACTCTCGGACACTCTT
OAZ1_1413_INS15
GGGAGGATTTCACCATCCGGAAAAAAAAAAGACGCTAATCCAGGATCT
OAZ1_1414_INS15
GCTATTGAGGATCCGCTGCAAAAAAAAAAACTTCTGCAGGACACTCTT
OAZ1_1415_INS15
ACAAACCCAGGCGAGATGAGAAAAAAAAAAGACGCTAATCCAGGATCT
OAZ1_1416_INS15
TGCACGATTACAACATGCGGAAAAAAAAAACTTCTGCAGGACACTCTT
OAZ1_1417_INS15
CTCTCTCGAACGTGTAGGCCAAAAAAAAAAGACGCTAATCCAGGATCT
OAZ1_1418_INS15
CTACTCCTCCTCCTCTCCCGAAAAAAAAAACTTCTGCAGGACACTCTT
OAZ1_1419_INS15
CCTTCCTTCTCTCTGGCGAAAAAAAAAAAAGACGCTAATCCAGGATCT
OAZ1_1420_INS15
ATGGTGGCGCTGGGTTTATCAAAAAAAAAACTTCTGCAGGACACTCTT
NAP1L1_1423_INS26
ATCCTGCTTCACTGCTGCTTAAAAAAAAAAGACGCTAATCCGCTAAGT
NAP1L1_1424_INS26
GCAGGTTATCCTCAAGGCCAAAAAAAAAAATCCTTCAGGGACACTCTT
NAP1L1_1425_INS26
TGCTTAGTGTCACACTGGCAAAAAAAAAAAGACGCTAATCCGCTAAGT
NAP1L1_1426_INS26
TCAAAGCGTGGTGTGAACAAAAAAAAAAAATCCTTCAGGGACACTCTT
NAP1L1_1427_1N526
CAGCACGTAGGGCCCTTTCAAAAAAAAAAGACGCTAATCCGCTAAGT
NAP1L1_1428_INS26
CCGCTCGCGCTCATATACAAAAAAAAAAAATCCTTCAGGGACACTCTT
NAP1L1_1429_1N526
TGGGCTGCTGGAAACTACAAAAAAAAAAAAGACGCTAATCCGCTAAGT
NAP1L1_1430_INS26
GAGACTTACTTGGCAGTGTGCAAAAAAAAAATCCTTCAGGGACACTCTT
HINT1_1433_INS13
GACCTGAGCCTTGGCAATCTAAAAAAAAAAGACGCTAATCTCAATCGG
HINT1_1434_INS13
AAAGATCGTGTCGCCACCAGAAAAAAAAAATTCTCCAGGGACACTCTT
HINT1_1435_INS13
TTTGCCGACCTCCAAGAACAAAAAAAAAAAGACGCTAATCTCAATCGG
HINT1_1436_INS13
CCCAAAACGTGCTTAACCAGGAAAAAAAAAATTCTCCAGGGACACTCTT
HINT1_1437_INS13
ATTCAGGCCCAGATCAGCAGAAAAAAAAAAGACGCTAATCTCAATCGG
HINT1_1438_INS13
ACCTTCATTCACCACCATTCGAAAAAAAAAAATTCTCCAGGGACACTCTT
HINT1_1441_INS13
TCGATAACCCTTATTCAGGCCCAAAAAAAAAAGACGCTAATCTCAATCGG
HINT1_1442_INS13
TCTGAACCTTCATTCACCACCAAAAAAAAAAATTCTCCAGGGACACTCTT
CXCR4_1465_INS25
CCAGACGCCAACATAGACCAAAAAAAAAAAGACGCTAATCAATTCCGG
CXCR4_1466_INS25
GGAATAGTCAGCAGGAGGGCAAAAAAAAAATTCGCACTGGACACTCTT
CXCR4_1467_INS25
AAGAACTTGGCCACAGGTCCAAAAAAAAAAGACGCTAATCAATTCCGG
CXCR4_1468_INS25
ACCACGAGACATACAGCAACTAAAAAAAAAATTCGCACTGGACACTCTT
CXCR4_1469_INS25
AAGTAGTGGGCTAAGGGCACAAAAAAAAAAGACGCTAATCAATTCCGG
CXCR4_1470_INS25
TCAGGCTTGCTTTCTTCAGGAAAAAAAAAAATTCGCACTGGACACTCTT
CXCR4_1471_INS25
TCATAGTCCCCTGAGCCCATAAAAAAAAAAGACGCTAATCAATTCCGG
CXCR4_1472_INS25
ACGGAAACAGGGTTCCTTCAAAAAAAAAAATTCGCACTGGACACTCTT
TNF_2057_INS7
TCGGGGTTCGAGAAGATGATCTGAAAAAAAAAAAGACGCTAATCTACATG
GC
TNF_2058_INS7
GAGGGTTTGCTACAACATGGGCTAAAAAAAAAAACCGCTTATGGACACTC
TT
TNF_2059_INS7
CTCACAGGGCAATGATCCCAAAGTAAAAAAAAAAGACGCTAATCTACATG
GC
TNF_2060_INS7
TTTGGGAAGGTTGGATGTTCGTCCAAAAAAAAAACCGCTTATGGACACTC
TT
TNF_2061_INS7
AAGTTCTAAGCTTGGGTTCCGACCAAAAAAAAAAGACGCTAATCTACATG
GC
TNF_2062_INS7
GTTTCGAAGTGGTGGTCTTGTTGCAAAAAAAAAACCGCTTATGGACACTC
TT
IL2_h_2691_INS6
GGCAGGAGTTGAGGTTACTGTGAGAAAAAAAAAAGACGCTAATCCAGACT
GT
IL2_h_2692_INS6
GCAAGACAGGAGTTGCATCCTGTAAAAAAAAAAACTTAGCCTGGACACTC
TT
IL2_h_2693_INS6
TGTGACAAGTGCAAGACTTAGTGCAAAAAAAAAAAGACGCTAATCCAGAC
TGT
IL2_h_2694_INS6
TGTAGAACTTGAAGTAGGTGCACTGTAAAAAAAAAACTTAGCCTGGACAC
TCTT
IL2_h_2695_INS6
TGCTCCAGTTGTAGCTGTGTTTTCTAAAAAAAAAAGACGCTAATCCAGAC
TGT
IL2_h_2696_INS6
AATGTGAGCATCCTGGTGAGTTTGAAAAAAAAAACTTAGCCTGGACACTC
TT
IL2_h_2697_INS6
TGAAGATGTTTCAGTTCTGTGGCCTAAAAAAAAAAGACGCTAATCCAGAC
TGT
IL2_h_2698_INS6
CCTCCAGAGGTTTGAGTTCTTCTTCTAAAAAAAAAACTTAGCCTGGACAC
TCTT
CSF2_h_2591_INS14
TCTCTACTCAGGTTCAGGAGACGCAAAAAAAAAAGACGCTAATCAGATGC
CT
CSF2_h_2592_INS14
ACTGTTTCATTCATCTCAGCAGCAAAAAAAAAAACACTTGTCGGACACTC
TT
CSF2_h_2593_INS14
GCTCTTGTTTCATGAGAGAGCAGCAAAAAAAAAAGACGCTAATCAGATGC
CT
CSF2_h_2594_INS14
TCCCTCCAAGATGACCATCCTGAGAAAAAAAAAACACTTGTCGGACACTC
TT
CSF2_h_2595_INS14
AGCAGAAAGTCCTTCAGGTTCTCTTAAAAAAAAAAGACGCTAATCAGATG
CCT
CSF2_h_2596_INS14
CTCCCAGCAGTCAAAGGGGATGAAAAAAAAAACACTTGTCGGACACTCTT
CSF2_h_2597_INS14
CAGTGCTGCTTGTAGTGGCTGGAAAAAAAAAAGACGCTAATCAGATGCCT
CSF2_h_2598_INS14
AATCTGGGTTGCACAGGAAGTTTCAAAAAAAAAACACTTGTCGGACACTC
TT
CCL3_h_2581_INS24
ATGGTGCAGAGGAGGACAGCAAGAAAAAAAAAAGACGCTAATCAACGCGT
T
CCL3_h_2582_INS24
AAGTGATGCAGAGAACTGGTTGCAAAAAAAAAAACTCCGATTGGACACTC
TT
CCL3_h_2583_INS24
GAAATTCTGTGGAATCTGCCGGGAAAAAAAAAAAGACGCTAATCAACGCG
TT
CCL3_h_2584_INS24
GGCTGCTCGTCTCAAAGTAGTCAGAAAAAAAAAACTCCGATTGGACACTC
TT
CCL3_h_2585_INS24
CAGCTCCAGGTCGCTGACATATTTAAAAAAAAAAGACGCTAATCAACGCG
TT
CCL3_h_2586_INS24
CTCGAAGCTTCTGGACCCCTCAGAAAAAAAAAACTCCGATTGGACACTCT
T
CCL3_h_2587_INS24
GAGGTCACACGCATGTTCCCAAGAAAAAAAAAAGACGCTAATCAACGCGT
T
CCL3_h_2588_INS24
GCAACAACCAGTCCATAGAAGAGGTAAAAAAAAAACTCCGATTGGACACT
CTT
IFNG_2561_INS17
CGATGCAGAGCTGAAAAGCCAAGAAAAAAAAAAAGACGCTAATCTGGCAC
AT
IFNG_2562_INS17
GCAGTAACAGCCAAGAGAACCCAAAAAAAAAAAACGCATCTTGGACACTC
TT
IFNG_2563_INS17
AGTCAGCTTTTCGAAGTCATCTCGTAAAAAAAAAAGACGCTAATCTGGCA
CAT
IFNG_2564_INS17
TTGCGTTGGACATTCAAGTCAGTTAAAAAAAAAACGCATCTTGGACACTC
TT
IFNG_2565_INS17
GCCTTGTAATCACATAGCCTTGCCAAAAAAAAAAGACGCTAATCTGGCAC
AT
IFNG_2566_INS17
GCTTAGGTTGGCTGCCTAGTTGGAAAAAAAAAACGCATCTTGGACACTCT
T
IFNG_2567_INS17
ACCGGCAGTAACTGGATAGTATCACAAAAAAAAAAGACGCTAATCTGGCA
CAT
IFNG_2568_INS17
TGGCTCAGATTGCAGGCATATTTTAAAAAAAAAACGCATCTTGGACACTC
TT
IFNG_2569_INS17
TTGGAAGCACCAGGCATGAAATCTAAAAAAAAAAGACGCTAATCTGGCAC
AT
IFNG_2570_INS17
TGGGTACAGTCACAGTTGTCAACAAAAAAAAAAACGCATCTTGGACACTC
TT
CCL4_2101_INS22
CAGTCACGCAGAGCTTCATGGTATAAAAAAAAAAGACGCTAATCATCAGC
GT
CCL4_2102_INS22
AGAAGGCAGCTACTAGCATGAGGAAAAAAAAAAACCATTCGTGGACACTC
TT
CCL4_2103_INS22
TCAGGTGACCTTCCCTGAAGACTTAAAAAAAAAAGACGCTAATCATCAGC
GT
CCL4_2104_INS22
ATGTGTCTCATGGAGAAGCATCCGAAAAAAAAAACCATTCGTGGACACTC
TT
CCL4_2105_INS22
CCATAGGGGACACTTATCCTTTGGCAAAAAAAAAAGACGCTAATCATCAG
CGT
CCL4_2106_INS22
ACAGCAGAGAAACAGTGACAGTGGAAAAAAAAAACCATTCGTGGACACTC
TT
PRF1_h_2641_INS8
GTGCTGAAGCTGTACTGGTCCTGAAAAAAAAAAGACGCTAATCAACCTGG
T
PRF1_h_2642_INS8
GAAACTGTAGAAGCGGCACTCCACAAAAAAAAAACTCGATCTGGACACTC
TT
PRF1_h_2643_INS8
TTCAGGTTGCATCTCACCTCATGGAAAAAAAAAAGACGCTAATCAACCTG
GT
PRF1_h_2644_INS8
TGATAGCGGAATTTTAGGTGGCCAAAAAAAAAAACTCGATCTGGACACTC
TT
PRF1_h_2645_INS8
TTCCAAGCTCACTGTTCTCACCACAAAAAAAAAAGACGCTAATCAACCTG
GT
PRF1_h_2646_INS8
CCCTTCAGTCCAAGCATACTGGTCAAAAAAAAAACTCGATCTGGACACTC
TT
PRF1_h_2647_INS8
CTGTGAGAACCCCTTCAGTCCAAGAAAAAAAAAAGACGCTAATCAACCTG
GT
PRF1_h_2648_INS8
AATGGGAATACGAAGACAGCCCTGAAAAAAAAAACTCGATCTGGACACTC
TT
CCL2_2129_INS1
AAGTCTTCGGAGTTTGGGTTTGCTAAAAAAAAAAGACGCTAATATCGTGA
CC
CCL2_2130_INS1
GCAGATTCTTGGGTTGTGGAGTGAAAAAAAAAAACTCAGTCGTGACACTC
TT
CCL2_2131_INS1
AGCCTCTGCACTGAGATCTTCCTAAAAAAAAAAAGACGCTAATATCGTGA
CC
CCL2_2132_INS1
TGCTGGTGATTCTTCTATAGCTCGCAAAAAAAAAACTCAGTCGTGACACT
CTT
CCL2_2133_INS1
GAGAGTGCGAGCTTCAGTTTGAGAAAAAAAAAAAGACGCTAATATCGTGA
CC
CCL2_2134_INS1
GGCAGAGACTTTCATGCTGGAGGAAAAAAAAAACTCAGTCGTGACACTCT
T
GZMB_h_2601_INS3
CGAAGTCGTCTCGTATCAGGAAGCAAAAAAAAAAGACGCTAATCTGCCAA
TG
GZMB_h_2602_INS3
TTATGGAGCTTCCCCAACAGTGAGAAAAAAAAAAGATTCCTCGGACACTC
TT
GZMB_h_2603_INS3
AGTGTGTGTGAGTGTTTTCCCAGGAAAAAAAAAAGACGCTAATCTGCCAA
TG
GZMB_h_2604_INS3
TCCTGCACTGTCATCTTCACCTCTAAAAAAAAAAGATTCCTCGGACACTC
TT
GZMB_h_2605_INS3
GCGTAAGTCAGATTCGCACTTTCGAAAAAAAAAAGACGCTAATCTGCCAA
TG
GZMB_h_2606_INS3
CGCACAACTCAATGGTACTGTCGTAAAAAAAAAAGATTCCTCGGACACTC
TT
GZMB_h_2607_INS3
GGCATGCCATTGTTTCGTCCATAGAAAAAAAAAAGACGCTAATCTGCCAA
TG
GZMB_h_2608_INS3
CAAAGCTTGAGACTTTGGTGCAGGAAAAAAAAAAGATTCCTCGGACACTC
TT
CXCL8_2067_INS14
TCACACAGAGCTGCAGAAATCAGGAAAAAAAAAAGACGCTAATCAGATGC
CT
CXCL8_2068_INS14
TTTAGCACTCCTTGGCAAAACTGCAAAAAAAAAACACTTGTCGGACACTC
TT
CXCL8_2069_INS14
TTGTGGATCCTGGCTAGCAGACTAAAAAAAAAAAGACGCTAATCAGATGC
CT
CXCL8_2070_INS14
AGAAACCAAGGCACAGTGGAACAAAAAAAAAAAACACTTGTCGGACACTC
TT
CXCL8_2071_INS14
TCCAGACAGAGCTCTCTTCCATCAAAAAAAAAAAGACGCTAATCAGATGC
CT
CXCL8_2072_INS14
AAACTTCTCCACAACCCTCTGCACAAAAAAAAAACACTTGTCGGACACTC
TT
Primers used in FIG. 8
CD44_mouse_2121_INS25
CGTAGGCACTACACCCCAATCTTCAAAAAAAAAAGACGCTAATCAATTCC
GG
CD44_mouse_2122_INS25
ACGTCTCCAATCGTGCTGTCTTTTAAAAAAAAAATTCGCACTGGACACTC
TT
CD44_mouse_2123_INS25
TAGCATACCCTGGTAATGCAAGGCAAAAAAAAAAGACGCTAATCAATTCC
GG
CD44_mouse_2124_INS25
AGGGACCAAGAATGTGCATTTGGTAAAAAAAAAATTCGCACTGGACACTC
TT
CD44_mouse_2125_INS25
AGTGCTCCTGTCCCTGATCTTCAAAAAAAAAAAAGACGCTAATCAATTCC
GG
CD44_mouse_2126_INS25
GAGAGAAACCCCTTTGAGAGCCTGAAAAAAAAAATTCGCACTGGACACTC
TT
CD44_mouse_2127_INS25
CCCTGGGACTTTGGGTAGGGATAAAAAAAAAAAAGACGCTAATCAATTCC
GG
CD44_mouse_2128_INS25
CTGGGGAAAATCCTTAGCTGGTGGAAAAAAAAAATTCGCACTGGACACTC
TT
MKI67_mouse_2141_INS14
CCTAGTTCCATCTGCTGCAGTCTGAAAAAAAAAAGACGCTAATCAGATGC
CT
MKI67_mouse_2142_INS14
CTTAGGTGTTTGTGGCTGTCTGGTAAAAAAAAAACACTTGTCGGACACTC
TT
MKI67_mouse_2143_INS14
AGGCATACCAGCTGTTGAATCCAGAAAAAAAAAAGACGCTAATCAGATGC
CT
MKI67_mouse_2144_INS14
ATCCTTTGAAGAACAGCGCATCCTAAAAAAAAAACACTTGTCGGACACTC
TT
MKI67_mouse_2145_INS14
TTCTCCAAGTGTGAGGGTTTGCATAAAAAAAAAAGACGCTAATCAGATGC
CT
MKI67_mouse_2146_INS14
CCCATCTTTGGTCTCTCTGCCATGAAAAAAAAAACACTTGTCGGACACTC
TT
MKI67_mouse_2147_INS14
ATCACTCATCTGCTGCTGCTTCTCAAAAAAAAAAGACGCTAATCAGATGC
CT
MKI67_mouse_2148_INS14
GCTGAATTGGAAAGTACGGAGCCTAAAAAAAAAACACTTGTCGGACACTC
TT
CDH1_mouse_2181_INS27
CAGTGTCCCTCCAAATCCGATACGAAAAAAAAAAGACGCTAATCTTAAGC
GC
CDH1_mouse_2182_INS27
AGTCTCTGGGTTAATCTCCAGCCAAAAAAAAAAACGTTACTCGGACACTC
TT
CDH1_mouse_2183_INS27
TGTTCTTCACATGCTCAGCGTCTTAAAAAAAAAAGACGCTAATCTTAAGC
GC
CDH1_mouse_2184_INS27
ATCATCTGTGGCGATGATGAGAGCAAAAAAAAAACGTTACTCGGACACTC
TT
CDH1_mouse_2185_INS27
ACCCACACCAAGATACCTGTCTCTAAAAAAAAAAGACGCTAATCTTAAGC
GC
CDH1_mouse_2186_INS27
TTGGGAACACACACACTATCCAGCAAAAAAAAAACGTTACTCGGACACTC
TT
CDH1_mouse_2187_INS27
GGAAGGTCTGGATCCAAGATGGTGAAAAAAAAAAGACGCTAATCTTAAGC
GC
CDH1_mouse_2188_INS27
GTTAGCTCAGCAGTAAAGGGGGACAAAAAAAAAACGTTACTCGGACACTC
TT
CD47_mouse_2191_INS24
CATTACGGACGATGCAAGGGATGAAAAAAAAAAAGACGCTAATCAACGCG
TT
CD47_mouse_2192_INS24
CAAACATTTCTTCGGTGCTTTGCGAAAAAAAAAACTCCGATTGGACACTC
TT
CD47_mouse_2193_INS24
GTCGTGAAGACCTGGTGCTTAGACAAAAAAAAAAGACGCTAATCAACGCG
TT
CD47_mouse_2194_INS24
CTGTTGTCTGTTCCTTCCAGCTGTAAAAAAAAAACTCCGATTGGACACTC
TT
CD47_mouse_2195_INS24
AAGGCTTGCTGGATACCACTGTTTAAAAAAAAAAGACGCTAATCAACGCG
TT
CD47_mouse_2196_INS24
AAAGCTGCTGGCAACCTGAGTTTAAAAAAAAAAACTCCGATTGGACACTC
TT
CD47_mouse_2197_INS24
GGATTTGCCCAACCACATTCGTTTAAAAAAAAAAGACGCTAATCAACGCG
TT
CD47_mouse_2198_INS24
ATATCATAGCACACAGAGGGGCCAAAAAAAAAAACTCCGATTGGACACTC
TT
KLF4_mouse_2031_INS8
TGAGCCCCAAAGTCAACGAAGATTAAAAAAAAAAGACGCTAATCAACCTG
GT
KLF4_mouse_2032_INS8
GAGTCCGAAGAAGAGAGAGGGGTAAAAAAAAAAACTCGATCTGGACACTC
TT
KLF4_mouse_2033_INS8
TAGGTCCAGGAGGTCGTTGAACTCAAAAAAAAAAGACGCTAATCAACCTG
GT
KLF4_mouse_2034_INS8
CTGGTGGGTTAGCGAGTTGGAAAGAAAAAAAAAACTCGATCTGGACACTC
TT
KLF4_mouse_2035_INS8
CCGGGCATGTTCAAGTTGGATTTGAAAAAAAAAAGACGCTAATCAACCTG
GT
KLF4_mouse_2036_INS8
CCAGTCACCCCTTGGCATTTTGTAAAAAAAAAAACTCGATCTGGACACTC
TT
KLF4_mouse_2037_INS8
ACTGAACTCTCTCTCCTGGCAGTGAAAAAAAAAAGACGCTAATCAACCTG
GT
KLF4_mouse_2038_INS8
CGTGGGAAGACAGTGTGAAAGGTTAAAAAAAAAACTCGATCTGGACACTC
TT
ESRRB_mouse_2071_INS16
GGTACACGATGCCCAAGATGAGAAAAAAAAAAAAGACGCTAATCTGTAGA
CC
ESRRB_mouse_2072_INS16
TATGCCAGCTTGTCATCGTATGGGAAAAAAAAAATCTATCCGGGACACTC
TT
ESRRB_mouse_2073_INS16
GCTGGGTCTCTCTGCTATCCTACAAAAAAAAAAAGACGCTAATCTGTAGA
CC
ESRRB_mouse_2074_INS16
TTCCGAGGTGCAATGAGACTTTCCAAAAAAAAAATCTATCCGGGACACTC
TT
ESRRB_mouse_2075_INS16
GAGGACTTGTCATGAAAGTGGCGTAAAAAAAAAAGACGCTAATCTGTAGA
CC
ESRRB_mouse_2076_INS16
GGAAGGGATCAGAGCAGGTAAAGCAAAAAAAAAATCTATCCGGGACACTC
TT
ESRRB_mouse_2077_INS16
GTGACCAGTCTCCTAGAGGTGTCAAAAAAAAAAAGACGCTAATCTGTAGA
CC
ESRRB_mouse_2078_INS16
GGCTCTCTGGGGAAGTTTAGCATTAAAAAAAAAATCTATCCGGGACACTC
TT
ACTB_mouse_2021_INS7
ACAGCTTCTCTTTGATGTCACGCAAAAAAAAAAAGACGCTAATCTACATG
GC
ACTB_mouse_2022_INS7
GCCATCTCCTGCTCGAAGTCTAGAAAAAAAAAAACCGCTTATGGACACTC
TT
ACTB_mouse_2023_INS7
ACGGATGTCAACGTCACACTTCATAAAAAAAAAAGACGCTAATCTACATG
GC
ACTB_mouse_2024_INS7
AGACAGCACTGTGTTGGCATAGAGAAAAAAAAAACCGCTTATGGACACTC
TT
ACTB_mouse_2025_INS7
CAATGCCTGGGTACATGGTGGTACAAAAAAAAAAGACGCTAATCTACATG
GC
ACTB_mouse_2026_INS7
GCCAGAGCAGTAATCTCCTTCTGCAAAAAAAAAACCGCTTATGGACACTC
TT
ACTB_mouse_2027_INS7
CCTGAGTCAAAAGCGCCAAAACAAAAAAAAAAAAGACGCTAATCTACATG
GC
ACTB_mouse_2028_INS7
TCGCCTTCACCGTTCCAGTTTTTAAAAAAAAAAACCGCTTATGGACACTC
TT
SOX2_mouse_2041_INS11
GCGCCTAACGTACCACTAGAACTTAAAAAAAAAAGACGCTAATCTCGGAA
TC
SOX2_mouse_2042_INS11
TAAAGACTTTTGCGAACTCCCTGCAAAAAAAAAATGACTCTCGGACACTC
TT
SOX2_mouse_2043_INS11
AGTCCCCCAAAAAGAAGTCCCAAGAAAAAAAAAAGACGCTAATCTCGGAA
TC
SOX2_mouse_2044_INS11
CCGCCCTCAGGTTTTCTCTGTACAAAAAAAAAATGACTCTCGGACACTCT
T
SOX2_mouse_2045_INS11
GCGTTAATTTGGATGGGATTGGTGGAAAAAAAAAAGACGCTAATCTCGGA
ATC
SOX2_mouse_2046_INS11
AGTTTTCTAGTCGGCATCACGGTTAAAAAAAAAATGACTCTCGGACACTC
TT
SOX2_mouse_2047_INS11
AATCTCTCCCCTTCTCCAGTTCGCAAAAAAAAAAGACGCTAATCTCGGAA
TC
SOX2_mouse_2048_INS11
ACCCCTCCCAATTCCCTTGTATCTAAAAAAAAAATGACTCTCGGACACTC
TT
Lincenc1_mouse_3011_INS15
AGAACTGGTACACAGCAAACCACAAAAAAAAAAAGACGCTAATCCAGGAT
CT
Lincenc1_mouse_3012_INS15
GAATCCACACTTCCCTAGGCCCTAAAAAAAAAAACTTCTGCAGGACACTC
TT
Lincenc1_mouse_3013_INS15
AAAGGTCACACCCAGCAAAGAACAAAAAAAAAAAGACGCTAATCCAGGAT
CT
Lincenc1_mouse_3014_INS15
TGGTTTCAGCATGGAACCCTGAAGAAAAAAAAAACTTCTGCAGGACACTC
TT
Lincenc1_mouse_3015_INS15
CTGTTGATCTAACCAGTGGCAGCAAAAAAAAAAAGACGCTAATCCAGGAT
CT
Lincenc1_mouse_3016_INS15
ATGCAGGCAAGGTTCAGTGTCTAGAAAAAAAAAACTTCTGCAGGACACTC
TT
Lincenc1_mouse_3017_INS15
TGACGTATGGAGATTGAGCTGTGCAAAAAAAAAAGACGCTAATCCAGGAT
CT
Lincenc1_mouse_3018_INS15
AAGACACTTTTGGACCGATCTGGCAAAAAAAAAACTTCTGCAGGACACTC
TT
ZFP42_mouse_2111_INS22
CGTCTTGCTTTAGGGTCAGTCTGTAAAAAAAAAAGACGCTAATCATCAGC
GT
ZFP42_mouse_2112_INS22
ACTCTGGTATTCTGGACTGGCCTTAAAAAAAAAACCATTCGTGGACACTC
TT
ZFP42_mouse_2113_INS22
GCTTCGTCCCCTTTGTCATGTACTAAAAAAAAAAGACGCTAATCATCAGC
GT
ZFP42_mouse_2114_INS22
TCTCTTGCGTGACCTCTCTCTTCTAAAAAAAAAACCATTCGTGGACACTC
TT
ZFP42_mouse_2115_INS22
ACTCTAGGTATCCGTCAGGGAAGCAAAAAAAAAAGACGCTAATCATCAGC
GT
ZFP42_mouse_2116_INS22
GGGTTCGGAAAACTCACCTCGTATAAAAAAAAAACCATTCGTGGACACTC
TT
ZFP42_mouse_2117_INS22
ACACTCCAGCATCGATAAGACACCAAAAAAAAAAGACGCTAATCATCAGC
GT
ZFP42_mouse_2118_INS22
TATCCCTCAGCTTCTTCTTGCACCAAAAAAAAAACCATTCGTGGACACTC
TT
SALL4_mouse_2091_INS19
GGAGTTATTGTTGGCCCCATGAGTAAAAAAAAAAGACGCTAATCGCCATG
AT
SALL4_mouse_2092_INS19
GTTCTCTATGGCCAGCTTCCTTCCAAAAAAAAAATCGCTACTGGACACTC
TT
SALL4_mouse_2093_INS19
GCTATGGTCACAAGCCACATCACTAAAAAAAAAAGACGCTAATCGCCATG
AT
SALL4_mouse_2094_INS19
ACTCTCGTGATTGTAGGATTGCCCAAAAAAAAAATCGCTACTGGACACTC
TT
SALL4_mouse_2095_INS19
CGCATTAGTCACCACAGAAGGACAAAAAAAAAAAGACGCTAATCGCCATG
AT
SALL4_mouse_2096_INS19
GGAGGCATAAAACCAGGTCCCTACAAAAAAAAAATCGCTACTGGACACTC
TT
SALL4_mouse_2097_INS19
AGGAAGCAGCAGGAGAAATTGTGGAAAAAAAAAAGACGCTAATCGCCATG
AT
SALL4_mouse_2098_INS19
GGAGGCATACTCTAAGGGCTCTGAAAAAAAAAAATCGCTACTGGACACTC
TT
CD9_mouse_2161_INS18
CACCTCATCCTTGTGGGTATAGCCAAAAAAAAAAGACGCTAATCCTCGAA
TG
CD9_mouse_2162_INS18
GGTGTCCTTGTAAAACTCCTGGAGTAAAAAAAAAATCTCACGTGGACACT
CTT
CD9_mouse_2163_INS18
GGGTTCATCCTTGCTCCGTAACTTAAAAAAAAAAGACGCTAATCCTCGAA
TG
CD9_mouse_2164_INS18
ATGGATGGCTTTGAGTGTTTCCCGAAAAAAAAAATCTCACGTGGACACTC
TT
CD9_mouse_2165_INS18
GGTGTCCGAGATAAACTGCTCCAAAAAAAAAAAAAGACGCTAATCCTCGA
ATG
CD9_mouse_2166_INS18
TTTCCAAAAGCTGTTTCTTGGGGCAAAAAAAAAATCTCACGTGGACACTC
TT
CD9_mouse_2167_INS18
GGTTGGGCAGACTCTAGACCATTTAAAAAAAAAAGACGCTAATCCTCGAA
TG
CD9_mouse_2168_INS18
GTCTTCAGGGCCGTTGTTCCTGAAAAAAAAAATCTCACGTGGACACTCTT
POU5F1_mouse_2051_INS13
TGGTTCCACCTTCTCCAACTTCACAAAAAAAAAAGACGCTAATCTCAATC
GG
POU5F1_mouse_2052_INS13
GCTTTCATGTCCTGGGACTCCTCAAAAAAAAAATTCTCCAGGGACACTCT
T
POU5F1_mouse_2053_INS13
AGGTTCTCATTGTTGTCGGCTTCCAAAAAAAAAAGACGCTAATCTCAATC
GG
POU5F1_mouse_2054_INS13
GGGTCTCCGATTTGCATATCTCCTGAAAAAAAAAATTCTCCAGGGACACT
CTT
POU5F1_mouse_2055_INS13
GATTGGCGATGTGAGTGATCTGCTAAAAAAAAAAGACGCTAATCTCAATC
GG
POU5F1_mouse_2056_INS13
AACCACATCCTTCTCTAGCCCAAGAAAAAAAAAATTCTCCAGGGACACTC
TT
POU5F1_mouse_2057_INS13
ACGGTTCTCAATGCTAGTTCGCTTAAAAAAAAAAGACGCTAATCTCAATC
GG
POU5F1_mouse_2058_INS13
GAAACATGGTCTCCAGACTCCACCAAAAAAAAAATTCTCCAGGGACACTC
TT
THY1_mouse_2001_INS1
GAAGCTCACAAAAGTAGTCGCCCTAAAAAAAAAAGACGCTAATATCGTGA
CC
THY1_mouse_2002_INS1
TTATTGGAGCTCATGGGATTCGCGAAAAAAAAAACTCAGTCGTGACACTC
TT
THY1_mouse_2003_INS1
TCTTTCAGGCATCTGGCTTGGTTGAAAAAAAAAAGACGCTAATATCGTGA
CC
THY1_mouse_2004_INS1
CACAGTCCAACTTCCCTCATCCATAAAAAAAAAACTCAGTCGTGACACTC
TT
THY1_mouse_2005_INS1
CCCCTCCCCGATGATTCTTTCAACAAAAAAAAAAGACGCTAATATCGTGA
CC
THY1_mouse_2006_INS1
TCAGCAGAGCTCTCCCATCTTGAGAAAAAAAAAACTCAGTCGTGACACTC
TT
THY1_mouse_2007_INS1
AGATCCTGGAGTCAGAGTTCTGGCAAAAAAAAAAGACGCTAATATCGTGA
CC
THY1_mouse_2008_INS1
ATGGTTCTAGGATCCCCTTCCTGCAAAAAAAAAACTCAGTCGTGACACTC
TT
NANOG_mouse_2011_INS4
CCTCAGAACTAGGCAAACTGTGGGAAAAAAAAAAGACGCTAATCAGGCTA
CT
NANOG_mouse_2012_INS4
GATGAGGCGTTCCCAGAATTCGATAAAAAAAAAACTACCTTGGGACACTC
TT
NANOG_mouse_2013_INS4
AGGCTGAGGTACTTCTGCTTCTGAAAAAAAAAAAGACGCTAATCAGGCTA
CT
NANOG_mouse_2014_INS4
ATGGAGGAGAGTTCTTGCATCTGCAAAAAAAAAACTACCTTGGGACACTC
TT
NANOG_mouse_2015_INS4
GCAGAGAAGTTTTGCTGCAACTGTAAAAAAAAAAGACGCTAATCAGGCTA
CT
NANOG_mouse_2016_INS4
AGTGGCTTCCAAATTCACCTCCAAAAAAAAAAAACTACCTTGGGACACTC
TT
NANOG_mouse_2017_INS4
AAGCCCAGATGTTGCGTAAGTCTCAAAAAAAAAAGACGCTAATCAGGCTA
CT
NANOG_mouse_2018_INS4
GGAAGAAGGAAGGAACCTGGCTTTAAAAAAAAAACTACCTTGGGACACTC
TT
PTPRC_1017_INS7
TTTTGCAATGATGTAGGCATGAAAAAAAAAAGACGCTAATCTACATGGC
PTPRC_1018_INS7
GCAGCACTTCCATTACGTTGAAAAAAAAAACCGCTTATGGACACTCTT
PTPRC_1021_INS7
TTCCAACAAAATATCTGCATGGAAAAAAAAAAGACGCTAATCTACATGGC
PTPRC_1022_INS7
CCTTCATCAGCAATCTTCCTCAAAAAAAAAACCGCTTATGGACACTCTT
PTPRC_1023_INS7
GAAACTTGCTGAACACCCGAAAAAAAAAAGACGCTAATCTACATGGC
PTPRC_1024_INS7
TAAAGGGCTTTCGAGCTTCCAAAAAAAAAACCGCTTATGGACACTCTT
PTPRC_1025_INS7
CAGTTTGAGGAGCAAGTGAGGAAAAAAAAAAGACGCTAATCTACATGGC
PTPRC_1026_INS7
GCTGAAGGCATTCACTCTCCAAAAAAAAAACCGCTTATGGACACTCTT
CD8A_1103_INS16
ATGTGATGTCACCCGAAGCAAAAAAAAAAGACGCTAATCTGTAGACC
CD8A_1104_INS16
GGAGCCTGATTTCGCATTTAAAAAAAAAATCTATCCGGGACACTCTT
CD8A_1105_INS16
CAACCTCTTGCCCGAGAACAAAAAAAAAAGACGCTAATCTGTAGACC
CD8A_1106_INS16
AGGGTGAGGACGAAGGTGTAAAAAAAAAATCTATCCGGGACACTCTT
CD8A_1107_INS16
TTGTCTCCCGATTTGACCACAAAAAAAAAAGACGCTAATCTGTAGACC
CD8A_1108_INS16
AGACGTATCTCGCCGAAAGGAAAAAAAAAATCTATCCGGGACACTCTT
CD8A_1111_1N516
GAACTCTGCGGGTAGCTCTGAAAAAAAAAAGACGCTAATCTGTAGACC
CD8A_1112_INS16
TCCAGCTCTCTCAGCATGATTAAAAAAAAAATCTATCCGGGACACTCTT
HLA-DRA_1141_INS20
CAGATAGAACTCGGCCTGGAAAAAAAAAAAGACGCTAATCACACTTGG
HLA-DRA_1142_INS20
TAAACTCGCCTGATTGGTCAAAAAAAAAAATACGCTCTGGACACTCTT
HLA-DRA_1143_INS20
TTGTCCACAGCTATGTTGGCAAAAAAAAAAGACGCTAATCACACTTGG
HLA-DRA_1144_INS20
CGCTTTGTCATGATTTCCAGAAAAAAAAAATACGCTCTGGACACTCTT
HLA-DRA_1145_INS20
GTGACATTGACCACTGGTGGAAAAAAAAAAGACGCTAATCACACTTGG
HLA-DRA_1146_INS20
CAGGTTTTCCATTTCGAAGCAAAAAAAAAATACGCTCTGGACACTCTT
HLA-DRA_1151_INS20
CGTTCTGCTGCATTGCTTTAAAAAAAAAAGACGCTAATCACACTTGG
HLA-DRA_1152_INS20
CTCCATGTGCCTTACAGAGGAAAAAAAAAATACGCTCTGGACACTCTT
ITGAX_1157_INS22
TGACAATGAGAATTTTGGCGAAAAAAAAAAGACGCTAATCATCAGCGT
ITGAX_1158_INS22
GCCTTCTTTCTTCCCATCAGAAAAAAAAAACCATTCGTGGACACTCTT
ITGAX_1159_INS22
AAACAGGTAGACAGCACCCCAAAAAAAAAAGACGCTAATCATCAGCGT
ITGAX_1160_INS22
ATGCTGGGTCCCAAGACTCAAAAAAAAAACCATTCGTGGACACTCTT
ITGAX_1161_INS22
ACTCGGACTCGGCTCAGACAAAAAAAAAAGACGCTAATCATCAGCGT
ITGAX_1162_INS22
TTTCACAGTGTGCCTTCAGCAAAAAAAAAACCATTCGTGGACACTCTT
ITGAX_1163_INS22
GATATGGTCGGCTCCACAGTAAAAAAAAAAGACGCTAATCATCAGCGT
ITGAX_1164_INS22
GAGATGCCGAGATTGTCCTGAAAAAAAAAACCATTCGTGGACACTCTT
Primers used in FIG. 9 (Flow Cytometry)
CCL4_2101_INS22
CAGTCACGCAGAGCTTCATGGTATAAAAAAAAAAGACGCTAATCATCAGC
GT
CCL4_2102_INS22
AGAAGGCAGCTACTAGCATGAGGAAAAAAAAAAACCATTCGTGGACACTC
TT
CCL4_2103_INS22
TCAGGTGACCTTCCCTGAAGACTTAAAAAAAAAAGACGCTAATCATCAGC
GT
CCL4_2104_INS22
ATGTGTCTCATGGAGAAGCATCCGAAAAAAAAAACCATTCGTGGACACTC
TT
CCL4_2105_INS22
CCATAGGGGACACTTATCCTTTGGCAAAAAAAAAAGACGCTAATCATCAG
CGT
CCL4_2106_INS22
ACAGCAGAGAAACAGTGACAGTGGAAAAAAAAAACCATTCGTGGACACTC
TT
TNF_2057_INS7
TCGGGGTTCGAGAAGATGATCTGAAAAAAAAAAAGACGCTAATCTACATG
GC
TNF_2058_INS7
GAGGGTTTGCTACAACATGGGCTAAAAAAAAAAACCGCTTATGGACACTC
TT
TNF_2059_INS7
CTCACAGGGCAATGATCCCAAAGTAAAAAAAAAAGACGCTAATCTACATG
GC
TNF_2060_INS7
TTTGGGAAGGTTGGATGTTCGTCCAAAAAAAAAACCGCTTATGGACACTC
TT
TNF_2061_INS7
AAGTTCTAAGCTTGGGTTCCGACCAAAAAAAAAAGACGCTAATCTACATG
GC
TNF_2062_INS7
GTTTCGAAGTGGTGGTCTTGTTGCAAAAAAAAAACCGCTTATGGACACTC
TT
CXCL8_2067_INS14
TCACACAGAGCTGCAGAAATCAGGAAAAAAAAAAGACGCTAATCAGATGC
CT
CXCL8_2068_INS14
TTTAGCACTCCTTGGCAAAACTGCAAAAAAAAAACACTTGTCGGACACTC
TT
CXCL8_2069_INS14
TTGTGGATCCTGGCTAGCAGACTAAAAAAAAAAAGACGCTAATCAGATGC
CT
CXCL8_2070_INS14
AGAAACCAAGGCACAGTGGAACAAAAAAAAAAAACACTTGTCGGACACTC
TT
CXCL8_2071_INS14
TCCAGACAGAGCTCTCTTCCATCAAAAAAAAAAAGACGCTAATCAGATGC
CT
CXCL8_2072_INS14
AAACTTCTCCACAACCCTCTGCACAAAAAAAAAACACTTGTCGGACACTC
TT
IL6_2083_INS1
CCTGGAGGGGAGATAGAGCTTCTCAAAAAAAAAAGACGCTAATATCGTGA
CC
IL6_2084_1N51
GCGCTTGTGGAGAAGGAGTTCATAAAAAAAAAAACTCAGTCGTGACACTC
TT
IL6_2085_1N51
TTCACCAGGCAAGTCTCCTCATTGAAAAAAAAAAGACGCTAATATCGTGA
CC
IL6_2086_1N51
ACCTCAAACTCCAAAAGACCAGTGAAAAAAAAAAACTCAGTCGTGACACT
CTT
IL6_2087_INS1
TCTGGCTTGTTCCTCACTACTCTCAAAAAAAAAAAGACGCTAATATCGTG
ACC
IL6_2088_INS1
GGACTTTTGTACTCATCTGCACAGCAAAAAAAAAACTCAGTCGTGACACT
CTT
Primers used in FIGS. 9 and 10 (Mass Cytometry)
CCL4_2101_INS22
CAGTCACGCAGAGCTTCATGGTATAAAAAAAAAAGACGCTAATCATCAGC
GT
CCL4_2102_INS22
AGAAGGCAGCTACTAGCATGAGGAAAAAAAAAAACCATTCGTGGACACTC
TT
CCL4_2103_INS22
TCAGGTGACCTTCCCTGAAGACTTAAAAAAAAAAGACGCTAATCATCAGC
GT
CCL4_2104_INS22
ATGTGTCTCATGGAGAAGCATCCGAAAAAAAAAACCATTCGTGGACACTC
TT
CCL4_2105_INS22
CCATAGGGGACACTTATCCTTTGGCAAAAAAAAAAGACGCTAATCATCAG
CGT
CCL4_2106_INS22
ACAGCAGAGAAACAGTGACAGTGGAAAAAAAAAACCATTCGTGGACACTC
TT
TNF_2057_INS7
TCGGGGTTCGAGAAGATGATCTGAAAAAAAAAAAGACGCTAATCTACATG
GC
TNF_2058_INS7
GAGGGTTTGCTACAACATGGGCTAAAAAAAAAAACCGCTTATGGACACTC
TT
TNF_2059_INS7
CTCACAGGGCAATGATCCCAAAGTAAAAAAAAAAGACGCTAATCTACATG
GC
TNF_2060_INS7
TTTGGGAAGGTTGGATGTTCGTCCAAAAAAAAAACCGCTTATGGACACTC
TT
TNF_2061_INS7
AAGTTCTAAGCTTGGGTTCCGACCAAAAAAAAAAGACGCTAATCTACATG
GC
TNF_2062_INS7
GTTTCGAAGTGGTGGTCTTGTTGCAAAAAAAAAACCGCTTATGGACACTC
TT
IL1B_2091_INS11
GTGCACATAAGCCTCGTTATCCCAAAAAAAAAAAGACGCTAATCTCGGAA
TC
IL1B_2092_INS11
GTGCAGTTCAGTGATCGTACAGGTAAAAAAAAAATGACTCTCGGACACTC
TT
IL1B_2095_INS11
CAACACGCAGGACAGGTACAGATTAAAAAAAAAAGACGCTAATCTCGGAA
TC
IL1B_2096_INS11
TCCAGCTGTAGAGTGGGCTTATCAAAAAAAAAAATGACTCTCGGACACTC
TT
IL1B_2097_INS11
GAAGACGGGCATGTTTTCTGCTTGAAAAAAAAAAGACGCTAATCTCGGAA
TC
IL1B_2098_INS11
GTCAGTTATATCCTGGCCGCCTTTAAAAAAAAAATGACTCTCGGACACTC
TT
CCL2_2129_INS1
AAGTCTTCGGAGTTTGGGTTTGCTAAAAAAAAAAGACGCTAATATCGTGA
CC
CCL2_2130_INS1
GCAGATTCTTGGGTTGTGGAGTGAAAAAAAAAAACTCAGTCGTGACACTC
TT
CCL2_2131_INS1
AGCCTCTGCACTGAGATCTTCCTAAAAAAAAAAAGACGCTAATATCGTGA
CC
CCL2_2132_INS1
TGCTGGTGATTCTTCTATAGCTCGCAAAAAAAAAACTCAGTCGTGACACT
CTT
CCL2_2133_INS1
GAGAGTGCGAGCTTCAGTTTGAGAAAAAAAAAAAGACGCTAATATCGTGA
CC
CCL2_2134_INS1
GGCAGAGACTTTCATGCTGGAGGAAAAAAAAAACTCAGTCGTGACACTCT
T
IL1A_2075_INS16
ACGCCAATGAAATGACTCCCTCTCAAAAAAAAAAGACGCTAATCTGTAGA
CC
IL1A_2076_INS16
TGGCCATCTTGACTTCTTTGCTGAAAAAAAAAAATCTATCCGGGACACTC
TT
IL1A_2079_INS16
AGAGGAGGTTGGTCTCACTACCTGAAAAAAAAAAGACGCTAATCTGTAGA
CC
IL1A_2080_INS16
GTTCTTAGTGCCGTGAGTTTCCCAAAAAAAAAAATCTATCCGGGACACTC
TT
IL1A_2081_INS16
AAGCACAACTTGGACCAAAATGCCAAAAAAAAAAGACGCTAATCTGTAGA
CC
IL1A_2082_INS16
AATGCAGAGTTTCCTGGCTATGGGAAAAAAAAAATCTATCCGGGACACTC
TT
IL6_2083_INS20
CCTGGAGGGGAGATAGAGCTTCTCAAAAAAAAAAGACGCTAATCACACTT
GG
IL6_2084_1N520
GCGCTTGTGGAGAAGGAGTTCATAAAAAAAAAAATACGCTCTGGACACTC
TT
IL6_2085_INS20
TTCACCAGGCAAGTCTCCTCATTGAAAAAAAAAAGACGCTAATCACACTT
GG
IL6_2086_INS20
ACCTCAAACTCCAAAAGACCAGTGAAAAAAAAAAATACGCTCTGGACACT
CTT
IL6_2087_INS20
TCTGGCTTGTTCCTCACTACTCTCAAAAAAAAAAAGACGCTAATCACACT
TGG
IL6_2088_INS20
GGACTTTTGTACTCATCTGCACAGCAAAAAAAAAATACGCTCTGGACACT
CTT
IL1RN_2111_INS4
CAGGTTGTTGTGACGCCTTCTGAGAAAAAAAAAAGACGCTAATCAGGCTA
CT
IL1RN_2112_INS4
GTCAGTTGAAGAGGAGGCAGAGTCAAAAAAAAAACTACCTTGGGACACTC
TT
IL1RN_2113_INS4
GGTAAAGTACTGCAGGCAGCTGTAAAAAAAAAAAGACGCTAATCAGGCTA
CT
IL1RN_2114_INS4
GAGCCTTGGGAGCTGAGAAACTTCAAAAAAAAAACTACCTTGGGACACTC
TT
IL1RN_2117_INS4
GGAAGGTGGAATGAGGGAGGAAGAAAAAAAAAAAGACGCTAATCAGGCTA
CT
IL1RN_2118_INS4
GTCATCAAGTGGCCTGATGGATCCAAAAAAAAAACTACCTTGGGACACTC
TT
CXCL8_2067_INS13
TCACACAGAGCTGCAGAAATCAGGAAAAAAAAAAGACGCTAATCTCAATC
GG
CXCL8_2068_INS13
TTTAGCACTCCTTGGCAAAACTGCAAAAAAAAAATTCTCCAGGGACACTC
TT
CXCL8_2069_INS13
TTGTGGATCCTGGCTAGCAGACTAAAAAAAAAAAGACGCTAATCTCAATC
GG
CXCL8_2070_INS13
AGAAACCAAGGCACAGTGGAACAAAAAAAAAAAATTCTCCAGGGACACTC
TT
CXCL8_2071_INS13
TCCAGACAGAGCTCTCTTCCATCAAAAAAAAAAAGACGCTAATCTCAATC
GG
CXCL8_2072_INS13
AAACTTCTCCACAACCCTCTGCACAAAAAAAAAATTCTCCAGGGACACTC
TT
Primers used in FIG. 12
ACTB_1057_INS8
GTCAGGCAGCTCGTAGCTCTAAAAAAAAAAGACGCTAATCAACCTGGT
ACTB_1058_INS8
TGCCAATGGTGATGACCTGAAAAAAAAAACTCGATCTGGACACTCTT
ACTB_1059_INS8
ATGTCCACGTCACACTTCATGAAAAAAAAAAGACGCTAATCAACCTGGT
ACTB_1060_INS8
TGTTGGCGTACAGGTCTTTGAAAAAAAAAACTCGATCTGGACACTCTT
ACTB_1063_INS8
ATCTGCTGGAAGGTGGACAGAAAAAAAAAAGACGCTAATCAACCTGGT
ACTB_1064_INS8
CGTCATACTCCTGCTTGCTGAAAAAAAAAACTCGATCTGGACACTCTT
ACTB_1065_INS8
TCAAGAAAGGGTGTAACGCAAAAAAAAAAAGACGCTAATCAACCTGGT
ACTB_1066_INS8
TGTTTTCTGCGCAAGTTAGGTAAAAAAAAAACTCGATCTGGACACTCTT
ACTB_1057_INS8_sense
AGAGCTACGAGCTGCCTGACAAAAAAAAAAGACGCTAATCAACCTGGT
ACTB_1058_1N58_sense
CAGGTCATCACCATTGGCAAAAAAAAAAACTCGATCTGGACACTCTT
ACTB_1059_INS8_sense
CATGAAGTGTGACGTGGACATAAAAAAAAAAGACGCTAATCAACCTGGT
ACTB_1060_INS8_sense
CAAAGACCTGTACGCCAACAAAAAAAAAAACTCGATCTGGACACTCTT
ACTB_1063_INS8_sense
CTGTCCACCTTCCAGCAGATAAAAAAAAAAGACGCTAATCAACCTGGT
ACTB_1064_INS8_sense
CAGCAAGCAGGAGTATGACGAAAAAAAAAACTCGATCTGGACACTCTT
ACTB_1065_INS8_sense
GCGTTACACCCTTTCTTGAAAAAAAAAAAAGACGCTAATCAACCTGGT
ACTB_1066_INS8_sense
ACCTAACTTGCGCAGAAAACAAAAAAAAAAACTCGATCTGGACACTCTT
ACTB_1056_INS8_PLAYR1
CACACGCAGCTCATTGTAGAAAAAAAAAAAGACGCTAATCAACCTGGT
ACTB_1058_INS8_PLAYR1
TGCCAATGGTGATGACCTGAAAAAAAAAAGACGCTAATCAACCTGGT
ACTB_1060_INS8_PLAYR1
TGTTGGCGTACAGGTCTTTGAAAAAAAAAAGACGCTAATCAACCTGGT
ACTB_1062_INS8_PLAYR1
TGATCTCCTTCTGCATCCTGAAAAAAAAAAGACGCTAATCAACCTGGT
ACTB_1064_INS8_PLAYR1
CGTCATACTCCTGCTTGCTGAAAAAAAAAAGACGCTAATCAACCTGGT
ACTB_1066_INS8_PLAYR1
TGTTTTCTGCGCAAGTTAGGTAAAAAAAAAAGACGCTAATCAACCTGGT
ACTB_1068_INS8_PLAYR1
GTGAACTTTGGGGGATGCTAAAAAAAAAAGACGCTAATCAACCTGGT
GAPDH_1030_INS8
CTTGAGGCCTGAGCTACGTGAAAAAAAAAACTCGATCTGGACACTCTT
GAPDH_1032_INS8
CAAAAGAAGATGCGGCTGACAAAAAAAAAACTCGATCTGGACACTCTT
GAPDH_1034_INS8
TGATGGCAACAATATCCACTTTAAAAAAAAAACTCGATCTGGACACTCTT
GAPDH_1036_INS8
TGGAAGATGGTGATGGGATTAAAAAAAAAACTCGATCTGGACACTCTT
Primers used in FIG. 13
PTPRC_213_INS1
TCTGTGTCCAGAAAGGCAAAGCCAAAAAAAAAAGACGCTAATATCGTGAC
C
PTPRC_214_INS1
GTGGGGGAAGGTGTTGGGCAAAAAAAAAACTCAGTCGTGACACTCTT
PTPRC_215_INS1
TCAGAGGCATTAAGGTAGGCATAAAAAAAAAAGACGCTAATATCGTGACC
PTPRC_216_INS1
GCTTCCAGAAGGGCTCAGAGTGAAAAAAAAAACTCAGTCGTGACACTCTT
PTPRC_217_INS1
CAGGAGCAGTACATGAATTATGAGAAAAAAAAAAAGACGCTAATATCGTG
ACC
PTPRC_218_INS1
TCAACCCCTGGTGGCACATCTAATAAAAAAAAAAACTCAGTCGTGACACT
CTT
ACTB_7_INS1
TTGCCAATGGTGATGACCTGAAAAAAAAAAGACGCTAATATCGTGACC
ACTB_8_INS1
GCCTCAGGGCAGCGGAACCGAAAAAAAAAACTCAGTCGTGACACTCTT
ACTB_157_INS1
GGGCGACGTAGCACAGCTAAAAAAAAAAGACGCTAATATCGTGACC
ACTB_158_INS1
CCGTGGCCATCTCTTGCTCGAAGTAAAAAAAAAACTCAGTCGTGACACTC
TT
ACTB_159_INS1
GGCCTCGGTCAGCAGCACAAAAAAAAAAGACGCTAATATCGTGACC
ACTB_160_INS1
CGCGGTTGGCCTTGGGGTTCAAAAAAAAAAACTCAGTCGTGACACTCTT
CD10_73_INS1
TGTCACAGCTATGATGGTGAGGAGAAAAAAAAAAGACGCTAATATCGTGA
CC
CD10_74_INS1
CATCGTAGGTTGCATAGAGTGCGATAAAAAAAAAACTCAGTCGTGACACT
CTT
CD10_75_INS1
CAACCAGCCTCCGCAAGCATATAAAAAAAAAAGACGCTAATATCGTGACC
CD10_76_INS1
CTGGTCTCGGGAATGACATTACGTAAAAAAAAAACTCAGTCGTGACACTC
TT
CD10_77_INS1
TGGATCAGTCGAGCAGCTGAAAAAAAAAAAGACGCTAATATCGTGACC
CD10_78_INS1
GTACAAGGCTCAGTGGTGGCATAAAAAAAAAACTCAGTCGTGACACTCTT
CD10_79_INS1
AGAATGCCGGCTGGGAAGACTAAAAAAAAAAAGACGCTAATATCGTGACC
CD10_80_INS1
GGACTGCTGGGCACTAAAGAAGGAAAAAAAAAACTCAGTCGTGACACTCT
T
CD10_81_INS1
CGTGTCCTATGACCATGCCGATGCCAAAAAAAAAAGACGCTAATATCGTG
ACC
CD10_82_INS1
GCCATTGTCATCGAAGCCATGGGTGAAAAAAAAAACTCAGTCGTGACACT
CTT
CD10_83_INS1
CCACACCTGTGCAAAGTTCAAGAAAAAAAAAAAAGACGCTAATATCGTGA
CC
CD10_84_INS1
CGCATACTCTGGCCTATAGGTTCCAAAAAAAAAAACTCAGTCGTGACACT
CTT
CD3E_93_INS1
AGGCCCAGAACTCTCCAGTAAAAAAAAAAGACGCTAATATCGTGACC
CD3E_94_INS1
CCAAACGCCAACTGATAAGAGGCAAAAAAAAAACTCAGTCGTGACACTCT
T
CD3E_95_INS1
ACTGTGGTTCCAGAGATGGAGACTAAAAAAAAAAGACGCTAATATCGTGA
CC
CD3E_96_INS1
CAGGATACTGAGGGCATGTCAATATAAAAAAAAAACTCAGTCGTGACACT
CTT
CD3E_97_INS1
TCCATCTCCATGCAGTTCTCACACAAAAAAAAAAAGACGCTAATATCGTG
ACC
CD3E_98_INS1
TGACAATTGTGGCCACCGACATCACAAAAAAAAAACTCAGTCGTGACACT
CTT
CD3E_99_INS1
CCCAGTGATGCAGATGTCCACTAAAAAAAAAAGACGCTAATATCGTGACC
CD3E_100_INS1
CAGTAGTAAACCAGCAGCAGCAAAAAAAAAAAACTCAGTCGTGACACTCT
T
CD3E_101_INS1
GAACAGGTGGTGGCCTCTCCAAAAAAAAAAGACGCTAATATCGTGACC
CD3E_102_INS1
TGGCCTTTCCGGATGGGCTCATAGTAAAAAAAAAACTCAGTCGTGACACT
CTT
HLA-DRA_243_INS14
CCACAGGGCTGTTTGTGAGCACAGAAAAAAAAAAGACGCTAATCAGATGC
CT
HLA-DRA_244_INS14
ATGAGGACGTTGGGCTCTCTCAGAAAAAAAAAACACTTGTCGGACACTCT
T
HLA-DRA_245_INS14
TGGGCAGGAAGACTGTCTCTGAAAAAAAAAAGACGCTAATCAGATGCCT
HLA-DRA_246_INS14
TGGAACTTGCGGAAAAGGTGGTCTAAAAAAAAAACACTTGTCGGACACTC
TT
HLA-DRA_247_INS14
CTCAGTTGAGGGCAGGAAGAAAAAAAAAAGACGCTAATCAGATGCCT
HLA-DRA_248_INS14
AGTGCTCCACCCTGCAGTCGTAAACAAAAAAAAAACACTTGTCGGACACT
CTT

What is claimed is:

1. A method for determining the abundance of a target nucleic acid in a single cell, the method comprising:
contacting a fixed and permeabilized cell with at least one pair of oligonucleotide primers under conditions permissive for specific hybridization, wherein each oligonucleotide in the pair comprises:
(i) a target binding region that hybridizes to the target nucleic acid;
(ii) a spacer region that does not bind to the target nucleic acid or to any region of a padlock probe; and
(iii) a PLAYR1 or PLAYR2 region that specifically binds to a padlock probe;
washing the cell free of unbound primer
contacting the cell with a padlock probe under conditions permissive for specific hybridization, wherein the padlock probe comprises separate polynucleotides of (i) a backbone and (ii) an insert;
contacting the cell with ligase wherein bound backbone and insert polynucleotides are ligated to generate a closed circle;
performing rolling circle amplification using the closed circle as a template and PLAYR1 or PLAYR2 as a primer for a polymerase;
contacting the cell with a detection probe under conditions permissive for specific hybridization; and
detecting the level of bound detection probes to determine the abundance of the target nucleic acid.

2. The method of claim 1, wherein the oligonucleotide primer pairs are denatured by heating before contacting the sample.

3. The method of claim 1, wherein the cell is present in a population of cells.

4. The method of claim 3, wherein the cell population comprises a plurality of cell types.

5. The method of claim 1, wherein a plurality of oligonucleotide primers are used.

6. The method of claim 5, wherein at least 5 different target nucleic acids are detected.

7. The method of claim 1, wherein the target nucleic acid is RNA.

8. The method of claim 7, wherein the RNA is mRNA.

9. The method of claim 1, wherein the target nucleic acid is DNA.

10. The method of claim 1, wherein the cell is simultaneously profiled for expression of one or more non-nucleic acid markers.

11. The method of claim 10, wherein the one or more markers are protein markers.

12. The method of any one of claim 1, wherein the detecting is performed by flow cytometry.

13. The method of claim 12, wherein the flow cytometry is mass cytometry or fluorescence-activated flow cytometry.

14. The method of any one of claim 1, wherein the detecting is performed by microscopy or nano-SIMS.

15. The method of claim 1, wherein each target binding region of a primer pair binds to a region of about 15-30 nucleotides of the target nucleic acid, wherein in a pair, each target site is different, and the target sites are adjacent on the target nucleic acid

16. The method of claim 13, wherein the pair of oligonucleotide primers are selected such that each primer in the pair has a similar melting temperature for binding to its cognate target site.

17. The method of claim 14, wherein the Tm is from about 50° C. to about 70° C.

18. The method of claim 15, wherein the Tm is from about 58° to about 62° C.

19. The method of claim 1, wherein the sequence of the PLAYR 1 and/or PLAYR 2 regions provides barcoding information for identification of the target nucleic acid for use in multiplex analysis.

20. A kit for use in the method of any one of claims 1.


SEQUENCE LISTING (579 sequences)

SEQ ID NO: 1 - DNA, 28 bp, Artificial sequence
  synthetic oligonucleotide
aaaaaaaaaactcagtcgtgacactctt

SEQ ID NO: 2 - DNA, 28 bp, Artificial sequence
  synthetic oligonucleotide
aaaaaaaaaactaccttgggacactctt

SEQ ID NO: 3 - DNA, 28 bp, Artificial sequence
  synthetic oligonucleotide
aaaaaaaaaaccgcttatggacactctt

SEQ ID NO: 4 - DNA, 28 bp, Artificial sequence
  synthetic oligonucleotide
aaaaaaaaaactcgatctggacactctt

SEQ ID NO: 5 - DNA, 28 bp, Artificial sequence
  synthetic oligonucleotide
aaaaaaaaaatgactctcggacactctt

SEQ ID NO: 6 - DNA, 28 bp, Artificial sequence
  synthetic oligonucleotide
aaaaaaaaaattctccagggacactctt

SEQ ID NO: 7 - DNA, 28 bp, Artificial sequence
  synthetic oligonucleotide
aaaaaaaaaacttctgcaggacactctt

SEQ ID NO: 8 - DNA, 28 bp, Artificial sequence
  synthetic oligonucleotide
aaaaaaaaaatctatccgggacactctt

SEQ ID NO: 9 - DNA, 28 bp, Artificial sequence
  synthetic oligonucleotide
aaaaaaaaaacgcatcttggacactctt

SEQ ID NO: 10 - DNA, 28 bp, Artificial sequence
  synthetic oligonucleotide
aaaaaaaaaatcgctactggacactctt

SEQ ID NO: 11 - DNA, 28 bp, Artificial sequence
  synthetic oligonucleotide
aaaaaaaaaatacgctctggacactctt

SEQ ID NO: 12 - DNA, 28 bp, Artificial sequence
  synthetic oligonucleotide
aaaaaaaaaaccattcgtggacactctt

SEQ ID NO: 13 - DNA, 28 bp, Artificial sequence
  synthetic oligonucleotide
aaaaaaaaaattcgcactggacactctt

SEQ ID NO: 14 - DNA, 28 bp, Artificial sequence
  synthetic oligonucleotide
aaaaaaaaaatccttcagggacactctt

SEQ ID NO: 15 - DNA, 28 bp, Artificial sequence
  synthetic oligonucleotide
aaaaaaaaaagacgctaatatcgtgacc

SEQ ID NO: 16 - DNA, 28 bp, Artificial sequence
  synthetic oligonucleotide
aaaaaaaaaagacgctaatcaggctact

SEQ ID NO: 17 - DNA, 28 bp, Artificial sequence
  synthetic oligonucleotide
aaaaaaaaaagacgctaatctacatggc

SEQ ID NO: 18 - DNA, 28 bp, Artificial sequence
  synthetic oligonucleotide
aaaaaaaaaagacgctaatcaacctggt

SEQ ID NO: 19 - DNA, 28 bp, Artificial sequence
  synthetic oligonucleotide
aaaaaaaaaagacgctaatctcggaatc

SEQ ID NO: 20 - DNA, 28 bp, Artificial sequence
  synthetic oligonucleotide
aaaaaaaaaagacgctaatctcaatcgg

SEQ ID NO: 21 - DNA, 28 bp, Artificial sequence
  synthetic oligonucleotide
aaaaaaaaaagacgctaatccaggatct

SEQ ID NO: 22 - DNA, 28 bp, Artificial sequence
  synthetic oligonucleotide
aaaaaaaaaagacgctaatctgtagacc

SEQ ID NO: 23 - DNA, 28 bp, Artificial sequence
  synthetic oligonucleotide
aaaaaaaaaagacgctaatctggcacat

SEQ ID NO: 24 - DNA, 28 bp, Artificial sequence
  synthetic oligonucleotide
aaaaaaaaaagacgctaatcgccatgat

SEQ ID NO: 25 - DNA, 28 bp, Artificial sequence
  synthetic oligonucleotide
aaaaaaaaaagacgctaatcacacttgg

SEQ ID NO: 26 - DNA, 28 bp, Artificial sequence
  synthetic oligonucleotide
aaaaaaaaaagacgctaatcatcagcgt

SEQ ID NO: 27 - DNA, 28 bp, Artificial sequence
  synthetic oligonucleotide
aaaaaaaaaagacgctaatcaattccgg

SEQ ID NO: 28 - DNA, 28 bp, Artificial sequence
  synthetic oligonucleotide
aaaaaaaaaagacgctaatccgctaagt

SEQ ID NO: 29 - DNA, 61 bp, Artificial sequence
  synthetic oligonucleotide
attagcgtccagtgaatgcgagtccgtctaggagagtagtacagcagccgtcaagagtgtc

SEQ ID NO: 30 - DNA, 21 bp, Artificial sequence
  synthetic oligonucleotide
acgactgagtttggtcacgat

SEQ ID NO: 31 - DNA, 21 bp, Artificial sequence
  synthetic oligonucleotide
ccaaggtagtttagtagcctg

SEQ ID NO: 32 - DNA, 21 bp, Artificial sequence
  synthetic oligonucleotide
cataagcggtttgccatgtag

SEQ ID NO: 33 - DNA, 21 bp, Artificial sequence
  synthetic oligonucleotide
cagatcgagtttaccaggttg

SEQ ID NO: 34 - DNA, 21 bp, Artificial sequence
  synthetic oligonucleotide
cgagagtcatttgattccgag

SEQ ID NO: 35 - DNA, 21 bp, Artificial sequence
  synthetic oligonucleotide
cctggagaatttccgattgag

SEQ ID NO: 36 - DNA, 21 bp, Artificial sequence
  synthetic oligonucleotide
ctgcagaagtttagatcctgg

SEQ ID NO: 37 - DNA, 21 bp, Artificial sequence
  synthetic oligonucleotide
ccggatagatttggtctacag

SEQ ID NO: 38 - DNA, 21 bp, Artificial sequence
  synthetic oligonucleotide
caagatgcgtttatgtgccag

SEQ ID NO: 39 - DNA, 21 bp, Artificial sequence
  synthetic oligonucleotide
cagtagcgatttatcatggcg

SEQ ID NO: 40 - DNA, 21 bp, Artificial sequence
  synthetic oligonucleotide
cagagcgtatttccaagtgtg

SEQ ID NO: 41 - DNA, 21 bp, Artificial sequence
  synthetic oligonucleotide
cacgaatggtttacgctgatg

SEQ ID NO: 42 - DNA, 21 bp, Artificial sequence
  synthetic oligonucleotide
cagtgcgaatttccggaattg

SEQ ID NO: 43 - DNA, 21 bp, Artificial sequence
  synthetic oligonucleotide
cctgaaggatttacttagcgg

SEQ ID NO: 44 - DNA, 20 bp, Artificial sequence
  synthetic oligonucleotide
cagtgaatgcgagtccgtct

SEQ ID NO: 45 - DNA, 21 bp, Artificial sequence
  synthetic oligonucleotide
acgactgagtttggtcacgat

SEQ ID NO: 46 - DNA, 21 bp, Artificial sequence
  synthetic oligonucleotide
ccaaggtagtttagtagcctg

SEQ ID NO: 47 - DNA, 21 bp, Artificial sequence
  synthetic oligonucleotide
cataagcggtttgccatgtag

SEQ ID NO: 48 - DNA, 21 bp, Artificial sequence
  synthetic oligonucleotide
cagatcgagtttaccaggttg

SEQ ID NO: 49 - DNA, 21 bp, Artificial sequence
  synthetic oligonucleotide
cgagagtcatttgattccgag

SEQ ID NO: 50 - DNA, 21 bp, Artificial sequence
  synthetic oligonucleotide
cctggagaatttccgattgag

SEQ ID NO: 51 - DNA, 21 bp, Artificial sequence
  synthetic oligonucleotide
ctgcagaagtttagatcctgg

SEQ ID NO: 52 - DNA, 21 bp, Artificial sequence
  synthetic oligonucleotide
ccggatagatttggtctacag

SEQ ID NO: 53 - DNA, 21 bp, Artificial sequence
  synthetic oligonucleotide
caagatgcgtttatgtgccag

SEQ ID NO: 54 - DNA, 21 bp, Artificial sequence
  synthetic oligonucleotide
cagtagcgatttatcatggcg

SEQ ID NO: 55 - DNA, 21 bp, Artificial sequence
  synthetic oligonucleotide
cagagcgtatttccaagtgtg

SEQ ID NO: 56 - DNA, 21 bp, Artificial sequence
  synthetic oligonucleotide
cacgaatggtttacgctgatg

SEQ ID NO: 57 - DNA, 21 bp, Artificial sequence
  synthetic oligonucleotide
cagtgcgaatttccggaattg

SEQ ID NO: 58 - DNA, 21 bp, Artificial sequence
  synthetic oligonucleotide
cctgaaggatttacttagcgg

SEQ ID NO: 59 - DNA, 52 bp, Artificial sequence
  synthetic oligonucleotide
ttcaagctccttggtaaacaggctaaaaaaaaaagacgctaatatcgtgacc

SEQ ID NO: 60 - DNA, 52 bp, Artificial sequence
  synthetic oligonucleotide
gtccacttcattcttctccagggcaaaaaaaaaactcagtcgtgacactctt

SEQ ID NO: 61 - DNA, 53 bp, Artificial sequence
  synthetic oligonucleotide
tgggtgaaagacaacagcatcatgaaaaaaaaaaagacgctaatatcgtgacc

SEQ ID NO: 62 - DNA, 52 bp, Artificial sequence
  synthetic oligonucleotide
tctggcagggtttctagggtcttcaaaaaaaaaactcagtcgtgacactctt

SEQ ID NO: 63 - DNA, 52 bp, Artificial sequence
  synthetic oligonucleotide
gaactccagatgcgggaactttctaaaaaaaaaagacgctaatatcgtgacc

SEQ ID NO: 64 - DNA, 52 bp, Artificial sequence
  synthetic oligonucleotide
ggtgttgaggtttccccgaatactaaaaaaaaaactcagtcgtgacactctt

SEQ ID NO: 65 - DNA, 52 bp, Artificial sequence
  synthetic oligonucleotide
ctaccaactgtgggtcatcctcagaaaaaaaaaagacgctaatatcgtgacc

SEQ ID NO: 66 - DNA, 52 bp, Artificial sequence
  synthetic oligonucleotide
tcgtggaatgttacgagcagtgataaaaaaaaaactcagtcgtgacactctt

SEQ ID NO: 67 - DNA, 52 bp, Artificial sequence
  synthetic oligonucleotide
cagatagcagtgagaatggggcacaaaaaaaaaagacgctaatatcgtgacc

SEQ ID NO: 68 - DNA, 52 bp, Artificial sequence
  synthetic oligonucleotide
ttcagtctcccggggtaatcactcaaaaaaaaaactcagtcgtgacactctt

SEQ ID NO: 69 - DNA, 52 bp, Artificial sequence
  synthetic oligonucleotide
gatcacccggcctacatcttcatcaaaaaaaaaagacgctaatctcaatcgg

SEQ ID NO: 70 - DNA, 52 bp, Artificial sequence
  synthetic oligonucleotide
ggaacagtctttccgaagagaccaaaaaaaaaaattctccagggacactctt

SEQ ID NO: 71 - DNA, 52 bp, Artificial sequence
  synthetic oligonucleotide
gctcaccgtagatgctctttcctcaaaaaaaaaagacgctaatctcaatcgg

SEQ ID NO: 72 - DNA, 52 bp, Artificial sequence
  synthetic oligonucleotide
tcagtttgaagttctcatcggggaaaaaaaaaaattctccagggacactctt

SEQ ID NO: 73 - DNA, 52 bp, Artificial sequence
  synthetic oligonucleotide
gtgatgaagaactgggagccgttgaaaaaaaaaagacgctaatctcaatcgg

SEQ ID NO: 74 - DNA, 52 bp, Artificial sequence
  synthetic oligonucleotide
catctagccaggctgtcttgactgaaaaaaaaaattctccagggacactctt

SEQ ID NO: 75 - DNA, 52 bp, Artificial sequence
  synthetic oligonucleotide
aaagggcttctccacctcgatcttaaaaaaaaaagacgctaatctcaatcgg

SEQ ID NO: 76 - DNA, 52 bp, Artificial sequence
  synthetic oligonucleotide
gaaagatgtccctgtgccctactcaaaaaaaaaattctccagggacactctt

SEQ ID NO: 77 - DNA, 52 bp, Artificial sequence
  synthetic oligonucleotide
caaaagtgagtccatgggcctgtgaaaaaaaaaagacgctaatctcaatcgg

SEQ ID NO: 78 - DNA, 52 bp, Artificial sequence
  synthetic oligonucleotide
tggtcagtgttggtaggagtttgtaaaaaaaaaattctccagggacactctt

SEQ ID NO: 79 - DNA, 51 bp, Artificial sequence
  synthetic oligonucleotide
tggttcacacccatgacgaacataaaaaaaaaagacgctaatcaggctact

SEQ ID NO: 80 - DNA, 52 bp, Artificial sequence
  synthetic oligonucleotide
tgctgatgatcttgaggctgttgtaaaaaaaaaactaccttgggacactctt

SEQ ID NO: 81 - DNA, 52 bp, Artificial sequence
  synthetic oligonucleotide
gactgtggtcatgagtccttccacaaaaaaaaaagacgctaatcaggctact

SEQ ID NO: 82 - DNA, 51 bp, Artificial sequence
  synthetic oligonucleotide
cagtcttctgggtggcagtgatgaaaaaaaaaactaccttgggacactctt

SEQ ID NO: 83 - DNA, 52 bp, Artificial sequence
  synthetic oligonucleotide
caggtttttctagacggcaggtcaaaaaaaaaaagacgctaatcaggctact

SEQ ID NO: 84 - DNA, 52 bp, Artificial sequence
  synthetic oligonucleotide
cctgcttcaccaccttcttgatgtaaaaaaaaaactaccttgggacactctt

SEQ ID NO: 85 - DNA, 52 bp, Artificial sequence
  synthetic oligonucleotide
gtccaggggtcttactccttggagaaaaaaaaaagacgctaatcaggctact

SEQ ID NO: 86 - DNA, 52 bp, Artificial sequence
  synthetic oligonucleotide
tctcttcctcttgtgctcttgctgaaaaaaaaaactaccttgggacactctt

SEQ ID NO: 87 - DNA, 52 bp, Artificial sequence
  synthetic oligonucleotide
tgtgaggaggggagattcagtgtgaaaaaaaaaagacgctaatcaggctact

SEQ ID NO: 88 - DNA, 52 bp, Artificial sequence
  synthetic oligonucleotide
cctcttcaaggggtctacatggcaaaaaaaaaaactaccttgggacactctt

SEQ ID NO: 89 - DNA, 50 bp, Artificial sequence
  synthetic oligonucleotide
tcgacctcgaaacagcatctgaaaaaaaaaaatgactctcggacactctt

SEQ ID NO: 90 - DNA, 52 bp, Artificial sequence
  synthetic oligonucleotide
caggacaaccattactgggatgctaaaaaaaaaagacgctaatctcggaatc

SEQ ID NO: 91 - DNA, 51 bp, Artificial sequence
  synthetic oligonucleotide
tcaaaccggcagtaactggatagaaaaaaaaaatgactctcggacactctt

SEQ ID NO: 92 - DNA, 53 bp, Artificial sequence
  synthetic oligonucleotide
aagcactggctcagattgcaggcataaaaaaaaaagacgctaatctcggaatc

SEQ ID NO: 93 - DNA, 51 bp, Artificial sequence
  synthetic oligonucleotide
agaacccaaaacgatgcagagctaaaaaaaaaatgactctcggacactctt

SEQ ID NO: 94 - DNA, 52 bp, Artificial sequence
  synthetic oligonucleotide
atatgggtcctggcagtaacagccaaaaaaaaaagacgctaatctcggaatc

SEQ ID NO: 95 - DNA, 52 bp, Artificial sequence
  synthetic oligonucleotide
tggaagcaccaggcatgaaatctcaaaaaaaaaatgactctcggacactctt

SEQ ID NO: 96 - DNA, 53 bp, Artificial sequence
  synthetic oligonucleotide
gggtacagtcacagttgtcaacaataaaaaaaaaagacgctaatctcggaatc

SEQ ID NO: 97 - DNA, 52 bp, Artificial sequence
  synthetic oligonucleotide
cagtcacgcagagcttcatggtataaaaaaaaaagacgctaatcatcagcgt

SEQ ID NO: 98 - DNA, 52 bp, Artificial sequence
  synthetic oligonucleotide
agaaggcagctactagcatgaggaaaaaaaaaaaccattcgtggacactctt

SEQ ID NO: 99 - DNA, 52 bp, Artificial sequence
  synthetic oligonucleotide
tcaggtgaccttccctgaagacttaaaaaaaaaagacgctaatcatcagcgt

SEQ ID NO: 100 - DNA, 52 bp, Artificial sequence
  synthetic oligonucleotide
atgtgtctcatggagaagcatccgaaaaaaaaaaccattcgtggacactctt

SEQ ID NO: 101 - DNA, 53 bp, Artificial sequence
  synthetic oligonucleotide
ccataggggacacttatcctttggcaaaaaaaaaagacgctaatcatcagcgt

SEQ ID NO: 102 - DNA, 52 bp, Artificial sequence
  synthetic oligonucleotide
acagcagagaaacagtgacagtggaaaaaaaaaaccattcgtggacactctt

SEQ ID NO: 103 - DNA, 48 bp, Artificial sequence
  synthetic oligonucleotide
ccactttgctccaattctgaaaaaaaaaaagacgctaatatcgtgacc

SEQ ID NO: 104 - DNA, 48 bp, Artificial sequence
  synthetic oligonucleotide
tcctctggggtagcagacataaaaaaaaaactcagtcgtgacactctt

SEQ ID NO: 105 - DNA, 48 bp, Artificial sequence
  synthetic oligonucleotide
gtaaaccagcagcagcaagcaaaaaaaaaagacgctaatatcgtgacc

SEQ ID NO: 106 - DNA, 49 bp, Artificial sequence
  synthetic oligonucleotide
ccttggcctttctattcttgcaaaaaaaaaactcagtcgtgacactctt

SEQ ID NO: 107 - DNA, 48 bp, Artificial sequence
  synthetic oligonucleotide
tggtggcctctccttgttttaaaaaaaaaagacgctaatatcgtgacc

SEQ ID NO: 108 - DNA, 49 bp, Artificial sequence
  synthetic oligonucleotide
ctcatagtctgggttgggaacaaaaaaaaaactcagtcgtgacactctt

SEQ ID NO: 109 - DNA, 48 bp, Artificial sequence
  synthetic oligonucleotide
cgtctctgattcaggccagaaaaaaaaaaagacgctaatatcgtgacc

SEQ ID NO: 110 - DNA, 49 bp, Artificial sequence
  synthetic oligonucleotide
cagtgttctccagagggtcagaaaaaaaaaactcagtcgtgacactctt

SEQ ID NO: 111 - DNA, 49 bp, Artificial sequence
  synthetic oligonucleotide
ttttgcaatgatgtaggcatgaaaaaaaaaagacgctaatctacatggc

SEQ ID NO: 112 - DNA, 48 bp, Artificial sequence
  synthetic oligonucleotide
gcagcacttccattacgttgaaaaaaaaaaccgcttatggacactctt

SEQ ID NO: 113 - DNA, 50 bp, Artificial sequence
  synthetic oligonucleotide
ttccaacaaaatatctgcatggaaaaaaaaaagacgctaatctacatggc

SEQ ID NO: 114 - DNA, 49 bp, Artificial sequence
  synthetic oligonucleotide
ccttcatcagcaatcttcctcaaaaaaaaaaccgcttatggacactctt

SEQ ID NO: 115 - DNA, 47 bp, Artificial sequence
  synthetic oligonucleotide
gaaacttgctgaacacccgaaaaaaaaaagacgctaatctacatggc

SEQ ID NO: 116 - DNA, 48 bp, Artificial sequence
  synthetic oligonucleotide
taaagggctttcgagcttccaaaaaaaaaaccgcttatggacactctt

SEQ ID NO: 117 - DNA, 49 bp, Artificial sequence
  synthetic oligonucleotide
cagtttgaggagcaagtgaggaaaaaaaaaagacgctaatctacatggc

SEQ ID NO: 118 - DNA, 48 bp, Artificial sequence
  synthetic oligonucleotide
gctgaaggcattcactctccaaaaaaaaaaccgcttatggacactctt

SEQ ID NO: 119 - DNA, 48 bp, Artificial sequence
  synthetic oligonucleotide
gttaaaagcagccctggtgaaaaaaaaaaagacgctaatcaggctact

SEQ ID NO: 120 - DNA, 50 bp, Artificial sequence
  synthetic oligonucleotide
tgatggcaacaatatccactttaaaaaaaaaactaccttgggacactctt

SEQ ID NO: 121 - DNA, 48 bp, Artificial sequence
  synthetic oligonucleotide
attgatgacaagcttcccgtaaaaaaaaaagacgctaatcaggctact

SEQ ID NO: 122 - DNA, 48 bp, Artificial sequence
  synthetic oligonucleotide
tggaagatggtgatgggattaaaaaaaaaactaccttgggacactctt

SEQ ID NO: 123 - DNA, 47 bp, Artificial sequence
  synthetic oligonucleotide
catcgccccacttgattttaaaaaaaaaagacgctaatcaggctact

SEQ ID NO: 124 - DNA, 48 bp, Artificial sequence
  synthetic oligonucleotide
tggactccacgacgtactcaaaaaaaaaaactaccttgggacactctt

SEQ ID NO: 125 - DNA, 50 bp, Artificial sequence
  synthetic oligonucleotide
tcatcatatttggcaggtttttaaaaaaaaaagacgctaatcaggctact

SEQ ID NO: 126 - DNA, 48 bp, Artificial sequence
  synthetic oligonucleotide
cctgcttcaccaccttcttgaaaaaaaaaactaccttgggacactctt

SEQ ID NO: 127 - DNA, 48 bp, Artificial sequence
  synthetic oligonucleotide
gtcaggcagctcgtagctctaaaaaaaaaagacgctaatcaacctggt

SEQ ID NO: 128 - DNA, 47 bp, Artificial sequence
  synthetic oligonucleotide
tgccaatggtgatgacctgaaaaaaaaaactcgatctggacactctt

SEQ ID NO: 129 - DNA, 49 bp, Artificial sequence
  synthetic oligonucleotide
atgtccacgtcacacttcatgaaaaaaaaaagacgctaatcaacctggt

SEQ ID NO: 130 - DNA, 48 bp, Artificial sequence
  synthetic oligonucleotide
tgttggcgtacaggtctttgaaaaaaaaaactcgatctggacactctt

SEQ ID NO: 131 - DNA, 48 bp, Artificial sequence
  synthetic oligonucleotide
atctgctggaaggtggacagaaaaaaaaaagacgctaatcaacctggt

SEQ ID NO: 132 - DNA, 48 bp, Artificial sequence
  synthetic oligonucleotide
cgtcatactcctgcttgctgaaaaaaaaaactcgatctggacactctt

SEQ ID NO: 133 - DNA, 48 bp, Artificial sequence
  synthetic oligonucleotide
tcaagaaagggtgtaacgcaaaaaaaaaaagacgctaatcaacctggt

SEQ ID NO: 134 - DNA, 49 bp, Artificial sequence
  synthetic oligonucleotide
tgttttctgcgcaagttaggtaaaaaaaaaactcgatctggacactctt

SEQ ID NO: 135 - DNA, 48 bp, Artificial sequence
  synthetic oligonucleotide
cagatagaactcggcctggaaaaaaaaaaagacgctaatcacacttgg

SEQ ID NO: 136 - DNA, 48 bp, Artificial sequence
  synthetic oligonucleotide
taaactcgcctgattggtcaaaaaaaaaaatacgctctggacactctt

SEQ ID NO: 137 - DNA, 48 bp, Artificial sequence
  synthetic oligonucleotide
ttgtccacagctatgttggcaaaaaaaaaagacgctaatcacacttgg

SEQ ID NO: 138 - DNA, 48 bp, Artificial sequence
  synthetic oligonucleotide
cgctttgtcatgatttccagaaaaaaaaaatacgctctggacactctt

SEQ ID NO: 139 - DNA, 48 bp, Artificial sequence
  synthetic oligonucleotide
gtgacattgaccactggtggaaaaaaaaaagacgctaatcacacttgg

SEQ ID NO: 140 - DNA, 48 bp, Artificial sequence
  synthetic oligonucleotide
caggttttccatttcgaagcaaaaaaaaaatacgctctggacactctt

SEQ ID NO: 141 - DNA, 47 bp, Artificial sequence
  synthetic oligonucleotide
cgttctgctgcattgctttaaaaaaaaaagacgctaatcacacttgg

SEQ ID NO: 142 - DNA, 48 bp, Artificial sequence
  synthetic oligonucleotide
ctccatgtgccttacagaggaaaaaaaaaatacgctctggacactctt

SEQ ID NO: 143 - DNA, 46 bp, Artificial sequence
  synthetic oligonucleotide
accacagcgtagagccgaaaaaaaaaaagacgctaatctgtagacc

SEQ ID NO: 144 - DNA, 49 bp, Artificial sequence
  synthetic oligonucleotide
gatgatgtagatgggctcctgaaaaaaaaaatctatccgggacactctt

SEQ ID NO: 145 - DNA, 48 bp, Artificial sequence
  synthetic oligonucleotide
ctcttcaatgaatgccatgcaaaaaaaaaagacgctaatctgtagacc

SEQ ID NO: 146 - DNA, 48 bp, Artificial sequence
  synthetic oligonucleotide
cccgaaggtcacgatgaataaaaaaaaaaatctatccgggacactctt

SEQ ID NO: 147 - DNA, 46 bp, Artificial sequence
  synthetic oligonucleotide
caatgaggcgtgctaggcaaaaaaaaaagacgctaatctgtagacc

SEQ ID NO: 148 - DNA, 48 bp, Artificial sequence
  synthetic oligonucleotide
ctccctggctgtgtactcgtaaaaaaaaaatctatccgggacactctt

SEQ ID NO: 149 - DNA, 46 bp, Artificial sequence
  synthetic oligonucleotide
acctccgggttggtcatcaaaaaaaaaagacgctaatctgtagacc

SEQ ID NO: 150 - DNA, 48 bp, Artificial sequence
  synthetic oligonucleotide
gtagcctcgctccaggttctaaaaaaaaaatctatccgggacactctt

SEQ ID NO: 151 - DNA, 48 bp, Artificial sequence
  synthetic oligonucleotide
agtggtacaccgtcttcccaaaaaaaaaaagacgctaatctggcacat

SEQ ID NO: 152 - DNA, 47 bp, Artificial sequence
  synthetic oligonucleotide
gccttgtcttggctgatgaaaaaaaaaaacgcatcttggacactctt

SEQ ID NO: 153 - DNA, 48 bp, Artificial sequence
  synthetic oligonucleotide
tatgaggaggttatcgcgctaaaaaaaaaagacgctaatctggcacat

SEQ ID NO: 154 - DNA, 47 bp, Artificial sequence
  synthetic oligonucleotide
cgcagccaagttcaatgtcaaaaaaaaaacgcatcttggacactctt

SEQ ID NO: 155 - DNA, 48 bp, Artificial sequence
  synthetic oligonucleotide
ctccaggtacttcatccccaaaaaaaaaaagacgctaatctggcacat

SEQ ID NO: 156 - DNA, 48 bp, Artificial sequence
  synthetic oligonucleotide
aggtcacggtgcacaaagttaaaaaaaaaacgcatcttggacactctt

SEQ ID NO: 157 - DNA, 48 bp, Artificial sequence
  synthetic oligonucleotide
ccatagctccagacatcgctaaaaaaaaaagacgctaatctggcacat

SEQ ID NO: 158 - DNA, 47 bp, Artificial sequence
  synthetic oligonucleotide
aggacaaggcctcccacataaaaaaaaaacgcatcttggacactctt

SEQ ID NO: 159 - DNA, 48 bp, Artificial sequence
  synthetic oligonucleotide
acacagtgccatcaacatggaaaaaaaaaagacgctaatcgccatgat

SEQ ID NO: 160 - DNA, 47 bp, Artificial sequence
  synthetic oligonucleotide
ctggcagtctgtggcagtgaaaaaaaaaatcgctactggacactctt

SEQ ID NO: 161 - DNA, 47 bp, Artificial sequence
  synthetic oligonucleotide
ctcatccgcatcctcacagaaaaaaaaaagacgctaatcgccatgat

SEQ ID NO: 162 - DNA, 48 bp, Artificial sequence
  synthetic oligonucleotide
gcctgggttgtgatagtcgtaaaaaaaaaatcgctactggacactctt

SEQ ID NO: 163 - DNA, 47 bp, Artificial sequence
  synthetic oligonucleotide
gctgagtgcaggagctgataaaaaaaaaagacgctaatcgccatgat

SEQ ID NO: 164 - DNA, 48 bp, Artificial sequence
  synthetic oligonucleotide
agaaggcactgtctcggatgaaaaaaaaaatcgctactggacactctt

SEQ ID NO: 165 - DNA, 48 bp, Artificial sequence
  synthetic oligonucleotide
cagttcctgggacacattcaaaaaaaaaaagacgctaatcgccatgat

SEQ ID NO: 166 - DNA, 48 bp, Artificial sequence
  synthetic oligonucleotide
ctcagtcttagccgctccagaaaaaaaaaatcgctactggacactctt

SEQ ID NO: 167 - DNA, 48 bp, Artificial sequence
  synthetic oligonucleotide
tggagaacaggaaggtgacaaaaaaaaaaagacgctaatcatcagcgt

SEQ ID NO: 168 - DNA, 48 bp, Artificial sequence
  synthetic oligonucleotide
tgcgagttccacacactgttaaaaaaaaaaccattcgtggacactctt

SEQ ID NO: 169 - DNA, 48 bp, Artificial sequence
  synthetic oligonucleotide
tcttggtggtaagggtgtgcaaaaaaaaaagacgctaatcatcagcgt

SEQ ID NO: 170 - DNA, 48 bp, Artificial sequence
  synthetic oligonucleotide
atggtgtgcaggacatctgaaaaaaaaaaaccattcgtggacactctt

SEQ ID NO: 171 - DNA, 48 bp, Artificial sequence
  synthetic oligonucleotide
agtggtcctcaatggacaggaaaaaaaaaagacgctaatcatcagcgt

SEQ ID NO: 172 - DNA, 48 bp, Artificial sequence
  synthetic oligonucleotide
atgtttctctgctgggcaataaaaaaaaaaccattcgtggacactctt

SEQ ID NO: 173 - DNA, 47 bp, Artificial sequence
  synthetic oligonucleotide
cctcagccagcttcttgtgaaaaaaaaaagacgctaatcatcagcgt

SEQ ID NO: 174 - DNA, 48 bp, Artificial sequence
  synthetic oligonucleotide
gtaggcacctcctcgtaggcaaaaaaaaaaccattcgtggacactctt

SEQ ID NO: 175 - DNA, 49 bp, Artificial sequence
  synthetic oligonucleotide
gcacaggctaatgttctgctgaaaaaaaaaagacgctaatctcggaatc

SEQ ID NO: 176 - DNA, 48 bp, Artificial sequence
  synthetic oligonucleotide
gagtgtgctccccaaacagaaaaaaaaaaatgactctcggacactctt

SEQ ID NO: 177 - DNA, 49 bp, Artificial sequence
  synthetic oligonucleotide
cgcacatctacagcaacaaggaaaaaaaaaagacgctaatctcggaatc

SEQ ID NO: 178 - DNA, 48 bp, Artificial sequence
  synthetic oligonucleotide
cttaaggctgccagactgcaaaaaaaaaaatgactctcggacactctt

SEQ ID NO: 179 - DNA, 48 bp, Artificial sequence
  synthetic oligonucleotide
cctgttatggcctgtggacaaaaaaaaaaagacgctaatctcggaatc

SEQ ID NO: 180 - DNA, 48 bp, Artificial sequence
  synthetic oligonucleotide
tgggttgcacgatgtctctcaaaaaaaaaatgactctcggacactctt

SEQ ID NO: 181 - DNA, 48 bp, Artificial sequence
  synthetic oligonucleotide
cagtgtgagacaaggtcccgaaaaaaaaaagacgctaatctcggaatc

SEQ ID NO: 182 - DNA, 48 bp, Artificial sequence
  synthetic oligonucleotide
gccggtcaacatgtgaggtaaaaaaaaaaatgactctcggacactctt

SEQ ID NO: 183 - DNA, 48 bp, Artificial sequence
  synthetic oligonucleotide
gggaggatttcaccatccggaaaaaaaaaagacgctaatccaggatct

SEQ ID NO: 184 - DNA, 48 bp, Artificial sequence
  synthetic oligonucleotide
gctattgaggatccgctgcaaaaaaaaaaacttctgcaggacactctt

SEQ ID NO: 185 - DNA, 48 bp, Artificial sequence
  synthetic oligonucleotide
acaaacccaggcgagatgagaaaaaaaaaagacgctaatccaggatct

SEQ ID NO: 186 - DNA, 48 bp, Artificial sequence
  synthetic oligonucleotide
tgcacgattacaacatgcggaaaaaaaaaacttctgcaggacactctt

SEQ ID NO: 187 - DNA, 48 bp, Artificial sequence
  synthetic oligonucleotide
ctctctcgaacgtgtaggccaaaaaaaaaagacgctaatccaggatct

SEQ ID NO: 188 - DNA, 48 bp, Artificial sequence
  synthetic oligonucleotide
ctactcctcctcctctcccgaaaaaaaaaacttctgcaggacactctt

SEQ ID NO: 189 - DNA, 48 bp, Artificial sequence
  synthetic oligonucleotide
ccttccttctctctggcgaaaaaaaaaaaagacgctaatccaggatct

SEQ ID NO: 190 - DNA, 48 bp, Artificial sequence
  synthetic oligonucleotide
atggtggcgctgggtttatcaaaaaaaaaacttctgcaggacactctt

SEQ ID NO: 191 - DNA, 48 bp, Artificial sequence
  synthetic oligonucleotide
atcctgcttcactgctgcttaaaaaaaaaagacgctaatccgctaagt

SEQ ID NO: 192 - DNA, 48 bp, Artificial sequence
  synthetic oligonucleotide
gcaggttatcctcaaggccaaaaaaaaaaatccttcagggacactctt

SEQ ID NO: 193 - DNA, 48 bp, Artificial sequence
  synthetic oligonucleotide
tgcttagtgtcacactggcaaaaaaaaaaagacgctaatccgctaagt

SEQ ID NO: 194 - DNA, 48 bp, Artificial sequence
  synthetic oligonucleotide
tcaaagcgtggtgtgaacaaaaaaaaaaaatccttcagggacactctt

SEQ ID NO: 195 - DNA, 47 bp, Artificial sequence
  synthetic oligonucleotide
cagcacgtagggccctttcaaaaaaaaaagacgctaatccgctaagt

SEQ ID NO: 196 - DNA, 48 bp, Artificial sequence
  synthetic oligonucleotide
ccgctcgcgctcatatacaaaaaaaaaaaatccttcagggacactctt

SEQ ID NO: 197 - DNA, 48 bp, Artificial sequence
  synthetic oligonucleotide
tgggctgctggaaactacaaaaaaaaaaaagacgctaatccgctaagt

SEQ ID NO: 198 - DNA, 49 bp, Artificial sequence
  synthetic oligonucleotide
gagacttacttggcagtgtgcaaaaaaaaaatccttcagggacactctt

SEQ ID NO: 199 - DNA, 48 bp, Artificial sequence
  synthetic oligonucleotide
gacctgagccttggcaatctaaaaaaaaaagacgctaatctcaatcgg

SEQ ID NO: 200 - DNA, 48 bp, Artificial sequence
  synthetic oligonucleotide
aaagatcgtgtcgccaccagaaaaaaaaaattctccagggacactctt

SEQ ID NO: 201 - DNA, 48 bp, Artificial sequence
  synthetic oligonucleotide
tttgccgacctccaagaacaaaaaaaaaaagacgctaatctcaatcgg

SEQ ID NO: 202 - DNA, 49 bp, Artificial sequence
  synthetic oligonucleotide
cccaaaacgtgcttaaccaggaaaaaaaaaattctccagggacactctt

SEQ ID NO: 203 - DNA, 48 bp, Artificial sequence
  synthetic oligonucleotide
attcaggcccagatcagcagaaaaaaaaaagacgctaatctcaatcgg

SEQ ID NO: 204 - DNA, 50 bp, Artificial sequence
  synthetic oligonucleotide
accttcattcaccaccattcgaaaaaaaaaaattctccagggacactctt

SEQ ID NO: 205 - DNA, 50 bp, Artificial sequence
  synthetic oligonucleotide
tcgataacccttattcaggcccaaaaaaaaaagacgctaatctcaatcgg

SEQ ID NO: 206 - DNA, 50 bp, Artificial sequence
  synthetic oligonucleotide
tctgaaccttcattcaccaccaaaaaaaaaaattctccagggacactctt

SEQ ID NO: 207 - DNA, 48 bp, Artificial sequence
  synthetic oligonucleotide
ccagacgccaacatagaccaaaaaaaaaaagacgctaatcaattccgg

SEQ ID NO: 208 - DNA, 48 bp, Artificial sequence
  synthetic oligonucleotide
ggaatagtcagcaggagggcaaaaaaaaaattcgcactggacactctt

SEQ ID NO: 209 - DNA, 48 bp, Artificial sequence
  synthetic oligonucleotide
aagaacttggccacaggtccaaaaaaaaaagacgctaatcaattccgg

SEQ ID NO: 210 - DNA, 49 bp, Artificial sequence
  synthetic oligonucleotide
accacgagacatacagcaactaaaaaaaaaattcgcactggacactctt

SEQ ID NO: 211 - DNA, 48 bp, Artificial sequence
  synthetic oligonucleotide
aagtagtgggctaagggcacaaaaaaaaaagacgctaatcaattccgg

SEQ ID NO: 212 - DNA, 49 bp, Artificial sequence
  synthetic oligonucleotide
tcaggcttgctttcttcaggaaaaaaaaaaattcgcactggacactctt

SEQ ID NO: 213 - DNA, 48 bp, Artificial sequence
  synthetic oligonucleotide
tcatagtcccctgagcccataaaaaaaaaagacgctaatcaattccgg

SEQ ID NO: 214 - DNA, 48 bp, Artificial sequence
  synthetic oligonucleotide
acggaaacagggttccttcaaaaaaaaaaattcgcactggacactctt

SEQ ID NO: 215 - DNA, 52 bp, Artificial sequence
  synthetic oligonucleotide
tcggggttcgagaagatgatctgaaaaaaaaaaagacgctaatctacatggc

SEQ ID NO: 216 - DNA, 52 bp, Artificial sequence
  synthetic oligonucleotide
gagggtttgctacaacatgggctaaaaaaaaaaaccgcttatggacactctt

SEQ ID NO: 217 - DNA, 52 bp, Artificial sequence
  synthetic oligonucleotide
ctcacagggcaatgatcccaaagtaaaaaaaaaagacgctaatctacatggc

SEQ ID NO: 218 - DNA, 52 bp, Artificial sequence
  synthetic oligonucleotide
tttgggaaggttggatgttcgtccaaaaaaaaaaccgcttatggacactctt

SEQ ID NO: 219 - DNA, 52 bp, Artificial sequence
  synthetic oligonucleotide
aagttctaagcttgggttccgaccaaaaaaaaaagacgctaatctacatggc

SEQ ID NO: 220 - DNA, 52 bp, Artificial sequence
  synthetic oligonucleotide
gtttcgaagtggtggtcttgttgcaaaaaaaaaaccgcttatggacactctt

SEQ ID NO: 221 - DNA, 52 bp, Artificial sequence
  synthetic oligonucleotide
ggcaggagttgaggttactgtgagaaaaaaaaaagacgctaatccagactgt

SEQ ID NO: 222 - DNA, 52 bp, Artificial sequence
  synthetic oligonucleotide
gcaagacaggagttgcatcctgtaaaaaaaaaaacttagcctggacactctt

SEQ ID NO: 223 - DNA, 53 bp, Artificial sequence
  synthetic oligonucleotide
tgtgacaagtgcaagacttagtgcaaaaaaaaaaagacgctaatccagactgt

SEQ ID NO: 224 - DNA, 54 bp, Artificial sequence
  synthetic oligonucleotide
tgtagaacttgaagtaggtgcactgtaaaaaaaaaacttagcctggacactctt

SEQ ID NO: 225 - DNA, 53 bp, Artificial sequence
  synthetic oligonucleotide
tgctccagttgtagctgtgttttctaaaaaaaaaagacgctaatccagactgt

SEQ ID NO: 226 - DNA, 52 bp, Artificial sequence
  synthetic oligonucleotide
aatgtgagcatcctggtgagtttgaaaaaaaaaacttagcctggacactctt

SEQ ID NO: 227 - DNA, 53 bp, Artificial sequence
  synthetic oligonucleotide
tgaagatgtttcagttctgtggcctaaaaaaaaaagacgctaatccagactgt

SEQ ID NO: 228 - DNA, 54 bp, Artificial sequence
  synthetic oligonucleotide
cctccagaggtttgagttcttcttctaaaaaaaaaacttagcctggacactctt

SEQ ID NO: 229 - DNA, 52 bp, Artificial sequence
  synthetic oligonucleotide
tctctactcaggttcaggagacgcaaaaaaaaaagacgctaatcagatgcct

SEQ ID NO: 230 - DNA, 52 bp, Artificial sequence
  synthetic oligonucleotide
actgtttcattcatctcagcagcaaaaaaaaaaacacttgtcggacactctt

SEQ ID NO: 231 - DNA, 52 bp, Artificial sequence
  synthetic oligonucleotide
gctcttgtttcatgagagagcagcaaaaaaaaaagacgctaatcagatgcct

SEQ ID NO: 232 - DNA, 52 bp, Artificial sequence
  synthetic oligonucleotide
tccctccaagatgaccatcctgagaaaaaaaaaacacttgtcggacactctt

SEQ ID NO: 233 - DNA, 53 bp, Artificial sequence
  synthetic oligonucleotide
agcagaaagtccttcaggttctcttaaaaaaaaaagacgctaatcagatgcct

SEQ ID NO: 234 - DNA, 50 bp, Artificial sequence
  synthetic oligonucleotide
ctcccagcagtcaaaggggatgaaaaaaaaaacacttgtcggacactctt

SEQ ID NO: 235 - DNA, 50 bp, Artificial sequence
  synthetic oligonucleotide
cagtgctgcttgtagtggctggaaaaaaaaaagacgctaatcagatgcct

SEQ ID NO: 236 - DNA, 52 bp, Artificial sequence
  synthetic oligonucleotide
aatctgggttgcacaggaagtttcaaaaaaaaaacacttgtcggacactctt

SEQ ID NO: 237 - DNA, 51 bp, Artificial sequence
  synthetic oligonucleotide
atggtgcagaggaggacagcaagaaaaaaaaaagacgctaatcaacgcgtt

SEQ ID NO: 238 - DNA, 52 bp, Artificial sequence
  synthetic oligonucleotide
aagtgatgcagagaactggttgcaaaaaaaaaaactccgattggacactctt

SEQ ID NO: 239 - DNA, 52 bp, Artificial sequence
  synthetic oligonucleotide
gaaattctgtggaatctgccgggaaaaaaaaaaagacgctaatcaacgcgtt

SEQ ID NO: 240 - DNA, 52 bp, Artificial sequence
  synthetic oligonucleotide
ggctgctcgtctcaaagtagtcagaaaaaaaaaactccgattggacactctt

SEQ ID NO: 241 - DNA, 52 bp, Artificial sequence
  synthetic oligonucleotide
cagctccaggtcgctgacatatttaaaaaaaaaagacgctaatcaacgcgtt

SEQ ID NO: 242 - DNA, 51 bp, Artificial sequence
  synthetic oligonucleotide
ctcgaagcttctggacccctcagaaaaaaaaaactccgattggacactctt

SEQ ID NO: 243 - DNA, 51 bp, Artificial sequence
  synthetic oligonucleotide
gaggtcacacgcatgttcccaagaaaaaaaaaagacgctaatcaacgcgtt

SEQ ID NO: 244 - DNA, 53 bp, Artificial sequence
  synthetic oligonucleotide
gcaacaaccagtccatagaagaggtaaaaaaaaaactccgattggacactctt

SEQ ID NO: 245 - DNA, 52 bp, Artificial sequence
  synthetic oligonucleotide
cgatgcagagctgaaaagccaagaaaaaaaaaaagacgctaatctggcacat

SEQ ID NO: 246 - DNA, 52 bp, Artificial sequence
  synthetic oligonucleotide
gcagtaacagccaagagaacccaaaaaaaaaaaacgcatcttggacactctt

SEQ ID NO: 247 - DNA, 53 bp, Artificial sequence
  synthetic oligonucleotide
agtcagcttttcgaagtcatctcgtaaaaaaaaaagacgctaatctggcacat

SEQ ID NO: 248 - DNA, 52 bp, Artificial sequence
  synthetic oligonucleotide
ttgcgttggacattcaagtcagttaaaaaaaaaacgcatcttggacactctt

SEQ ID NO: 249 - DNA, 52 bp, Artificial sequence
  synthetic oligonucleotide
gccttgtaatcacatagccttgccaaaaaaaaaagacgctaatctggcacat

SEQ ID NO: 250 - DNA, 51 bp, Artificial sequence
  synthetic oligonucleotide
gcttaggttggctgcctagttggaaaaaaaaaacgcatcttggacactctt

SEQ ID NO: 251 - DNA, 53 bp, Artificial sequence
  synthetic oligonucleotide
accggcagtaactggatagtatcacaaaaaaaaaagacgctaatctggcacat

SEQ ID NO: 252 - DNA, 52 bp, Artificial sequence
  synthetic oligonucleotide
tggctcagattgcaggcatattttaaaaaaaaaacgcatcttggacactctt

SEQ ID NO: 253 - DNA, 52 bp, Artificial sequence
  synthetic oligonucleotide
ttggaagcaccaggcatgaaatctaaaaaaaaaagacgctaatctggcacat

SEQ ID NO: 254 - DNA, 52 bp, Artificial sequence
  synthetic oligonucleotide
tgggtacagtcacagttgtcaacaaaaaaaaaaacgcatcttggacactctt

SEQ ID NO: 255 - DNA, 52 bp, Artificial sequence; identical to SEQ ID NO: 97
  synthetic oligonucleotide

SEQ ID NO: 256 - DNA, 52 bp, Artificial sequence; identical to SEQ ID NO: 98
  synthetic oligonucleotide

SEQ ID NO: 257 - DNA, 52 bp, Artificial sequence; identical to SEQ ID NO: 99
  synthetic oligonucleotide

SEQ ID NO: 258 - DNA, 52 bp, Artificial sequence; identical to SEQ ID NO: 100
  synthetic oligonucleotide

SEQ ID NO: 259 - DNA, 53 bp, Artificial sequence; identical to SEQ ID NO: 101
  synthetic oligonucleotide

SEQ ID NO: 260 - DNA, 52 bp, Artificial sequence; identical to SEQ ID NO: 102
  synthetic oligonucleotide

SEQ ID NO: 261 - DNA, 51 bp, Artificial sequence
  synthetic oligonucleotide
gtgctgaagctgtactggtcctgaaaaaaaaaagacgctaatcaacctggt

SEQ ID NO: 262 - DNA, 52 bp, Artificial sequence
  synthetic oligonucleotide
gaaactgtagaagcggcactccacaaaaaaaaaactcgatctggacactctt

SEQ ID NO: 263 - DNA, 52 bp, Artificial sequence
  synthetic oligonucleotide
ttcaggttgcatctcacctcatggaaaaaaaaaagacgctaatcaacctggt

SEQ ID NO: 264 - DNA, 52 bp, Artificial sequence
  synthetic oligonucleotide
tgatagcggaattttaggtggccaaaaaaaaaaactcgatctggacactctt

SEQ ID NO: 265 - DNA, 52 bp, Artificial sequence
  synthetic oligonucleotide
ttccaagctcactgttctcaccacaaaaaaaaaagacgctaatcaacctggt

SEQ ID NO: 266 - DNA, 52 bp, Artificial sequence
  synthetic oligonucleotide
cccttcagtccaagcatactggtcaaaaaaaaaactcgatctggacactctt

SEQ ID NO: 267 - DNA, 52 bp, Artificial sequence
  synthetic oligonucleotide
ctgtgagaaccccttcagtccaagaaaaaaaaaagacgctaatcaacctggt

SEQ ID NO: 268 - DNA, 52 bp, Artificial sequence
  synthetic oligonucleotide
aatgggaatacgaagacagccctgaaaaaaaaaactcgatctggacactctt

SEQ ID NO: 269 - DNA, 52 bp, Artificial sequence
  synthetic oligonucleotide
aagtcttcggagtttgggtttgctaaaaaaaaaagacgctaatatcgtgacc

SEQ ID NO: 270 - DNA, 52 bp, Artificial sequence
  synthetic oligonucleotide
gcagattcttgggttgtggagtgaaaaaaaaaaactcagtcgtgacactctt

SEQ ID NO: 271 - DNA, 52 bp, Artificial sequence
  synthetic oligonucleotide
agcctctgcactgagatcttcctaaaaaaaaaaagacgctaatatcgtgacc

SEQ ID NO: 272 - DNA, 53 bp, Artificial sequence
  synthetic oligonucleotide
tgctggtgattcttctatagctcgcaaaaaaaaaactcagtcgtgacactctt

SEQ ID NO: 273 - DNA, 52 bp, Artificial sequence
  synthetic oligonucleotide
gagagtgcgagcttcagtttgagaaaaaaaaaaagacgctaatatcgtgacc

SEQ ID NO: 274 - DNA, 51 bp, Artificial sequence
  synthetic oligonucleotide
ggcagagactttcatgctggaggaaaaaaaaaactcagtcgtgacactctt

SEQ ID NO: 275 - DNA, 52 bp, Artificial sequence
  synthetic oligonucleotide
cgaagtcgtctcgtatcaggaagcaaaaaaaaaagacgctaatctgccaatg

SEQ ID NO: 276 - DNA, 52 bp, Artificial sequence
  synthetic oligonucleotide
ttatggagcttccccaacagtgagaaaaaaaaaagattcctcggacactctt

SEQ ID NO: 277 - DNA, 52 bp, Artificial sequence
  synthetic oligonucleotide
agtgtgtgtgagtgttttcccaggaaaaaaaaaagacgctaatctgccaatg

SEQ ID NO: 278 - DNA, 52 bp, Artificial sequence
  synthetic oligonucleotide
tcctgcactgtcatcttcacctctaaaaaaaaaagattcctcggacactctt

SEQ ID NO: 279 - DNA, 52 bp, Artificial sequence
  synthetic oligonucleotide
gcgtaagtcagattcgcactttcgaaaaaaaaaagacgctaatctgccaatg

SEQ ID NO: 280 - DNA, 52 bp, Artificial sequence
  synthetic oligonucleotide
cgcacaactcaatggtactgtcgtaaaaaaaaaagattcctcggacactctt

SEQ ID NO: 281 - DNA, 52 bp, Artificial sequence
  synthetic oligonucleotide
ggcatgccattgtttcgtccatagaaaaaaaaaagacgctaatctgccaatg

SEQ ID NO: 282 - DNA, 52 bp, Artificial sequence
  synthetic oligonucleotide
caaagcttgagactttggtgcaggaaaaaaaaaagattcctcggacactctt

SEQ ID NO: 283 - DNA, 52 bp, Artificial sequence
  synthetic oligonucleotide
tcacacagagctgcagaaatcaggaaaaaaaaaagacgctaatcagatgcct

SEQ ID NO: 284 - DNA, 52 bp, Artificial sequence
  synthetic oligonucleotide
tttagcactccttggcaaaactgcaaaaaaaaaacacttgtcggacactctt

SEQ ID NO: 285 - DNA, 52 bp, Artificial sequence
  synthetic oligonucleotide
ttgtggatcctggctagcagactaaaaaaaaaaagacgctaatcagatgcct

SEQ ID NO: 286 - DNA, 52 bp, Artificial sequence
  synthetic oligonucleotide
agaaaccaaggcacagtggaacaaaaaaaaaaaacacttgtcggacactctt

SEQ ID NO: 287 - DNA, 52 bp, Artificial sequence
  synthetic oligonucleotide
tccagacagagctctcttccatcaaaaaaaaaaagacgctaatcagatgcct

SEQ ID NO: 288 - DNA, 52 bp, Artificial sequence
  synthetic oligonucleotide
aaacttctccacaaccctctgcacaaaaaaaaaacacttgtcggacactctt

SEQ ID NO: 289 - DNA, 52 bp, Artificial sequence
  synthetic oligonucleotide
cgtaggcactacaccccaatcttcaaaaaaaaaagacgctaatcaattccgg

SEQ ID NO: 290 - DNA, 52 bp, Artificial sequence
  synthetic oligonucleotide
acgtctccaatcgtgctgtcttttaaaaaaaaaattcgcactggacactctt

SEQ ID NO: 291 - DNA, 52 bp, Artificial sequence
  synthetic oligonucleotide
tagcataccctggtaatgcaaggcaaaaaaaaaagacgctaatcaattccgg

SEQ ID NO: 292 - DNA, 52 bp, Artificial sequence
  synthetic oligonucleotide
agggaccaagaatgtgcatttggtaaaaaaaaaattcgcactggacactctt

SEQ ID NO: 293 - DNA, 52 bp, Artificial sequence
  synthetic oligonucleotide
agtgctcctgtccctgatcttcaaaaaaaaaaaagacgctaatcaattccgg

SEQ ID NO: 294 - DNA, 52 bp, Artificial sequence
  synthetic oligonucleotide
gagagaaacccctttgagagcctgaaaaaaaaaattcgcactggacactctt

SEQ ID NO: 295 - DNA, 52 bp, Artificial sequence
  synthetic oligonucleotide
ccctgggactttgggtagggataaaaaaaaaaaagacgctaatcaattccgg

SEQ ID NO: 296 - DNA, 52 bp, Artificial sequence
  synthetic oligonucleotide
ctggggaaaatccttagctggtggaaaaaaaaaattcgcactggacactctt

SEQ ID NO: 297 - DNA, 52 bp, Artificial sequence
  synthetic oligonucleotide
cctagttccatctgctgcagtctgaaaaaaaaaagacgctaatcagatgcct

SEQ ID NO: 298 - DNA, 52 bp, Artificial sequence
  synthetic oligonucleotide
cttaggtgtttgtggctgtctggtaaaaaaaaaacacttgtcggacactctt

SEQ ID NO: 299 - DNA, 52 bp, Artificial sequence
  synthetic oligonucleotide
aggcataccagctgttgaatccagaaaaaaaaaagacgctaatcagatgcct

SEQ ID NO: 300 - DNA, 52 bp, Artificial sequence
  synthetic oligonucleotide
atcctttgaagaacagcgcatcctaaaaaaaaaacacttgtcggacactctt

SEQ ID NO: 301 - DNA, 52 bp, Artificial sequence
  synthetic oligonucleotide
ttctccaagtgtgagggtttgcataaaaaaaaaagacgctaatcagatgcct

SEQ ID NO: 302 - DNA, 52 bp, Artificial sequence
  synthetic oligonucleotide
cccatctttggtctctctgccatgaaaaaaaaaacacttgtcggacactctt

SEQ ID NO: 303 - DNA, 52 bp, Artificial sequence
  synthetic oligonucleotide
atcactcatctgctgctgcttctcaaaaaaaaaagacgctaatcagatgcct

SEQ ID NO: 304 - DNA, 52 bp, Artificial sequence
  synthetic oligonucleotide
gctgaattggaaagtacggagcctaaaaaaaaaacacttgtcggacactctt

SEQ ID NO: 305 - DNA, 52 bp, Artificial sequence
  synthetic oligonucleotide
cagtgtccctccaaatccgatacgaaaaaaaaaagacgctaatcttaagcgc

SEQ ID NO: 306 - DNA, 52 bp, Artificial sequence
  synthetic oligonucleotide
agtctctgggttaatctccagccaaaaaaaaaaacgttactcggacactctt

SEQ ID NO: 307 - DNA, 52 bp, Artificial sequence
  synthetic oligonucleotide
tgttcttcacatgctcagcgtcttaaaaaaaaaagacgctaatcttaagcgc

SEQ ID NO: 308 - DNA, 52 bp, Artificial sequence
  synthetic oligonucleotide
atcatctgtggcgatgatgagagcaaaaaaaaaacgttactcggacactctt

SEQ ID NO: 309 - DNA, 52 bp, Artificial sequence
  synthetic oligonucleotide
acccacaccaagatacctgtctctaaaaaaaaaagacgctaatcttaagcgc

SEQ ID NO: 310 - DNA, 52 bp, Artificial sequence
  synthetic oligonucleotide
ttgggaacacacacactatccagcaaaaaaaaaacgttactcggacactctt

SEQ ID NO: 311 - DNA, 52 bp, Artificial sequence
  synthetic oligonucleotide
ggaaggtctggatccaagatggtgaaaaaaaaaagacgctaatcttaagcgc

SEQ ID NO: 312 - DNA, 52 bp, Artificial sequence
  synthetic oligonucleotide
gttagctcagcagtaaagggggacaaaaaaaaaacgttactcggacactctt

SEQ ID NO: 313 - DNA, 52 bp, Artificial sequence
  synthetic oligonucleotide
cattacggacgatgcaagggatgaaaaaaaaaaagacgctaatcaacgcgtt

SEQ ID NO: 314 - DNA, 52 bp, Artificial sequence
  synthetic oligonucleotide
caaacatttcttcggtgctttgcgaaaaaaaaaactccgattggacactctt

SEQ ID NO: 315 - DNA, 52 bp, Artificial sequence
  synthetic oligonucleotide
gtcgtgaagacctggtgcttagacaaaaaaaaaagacgctaatcaacgcgtt

SEQ ID NO: 316 - DNA, 52 bp, Artificial sequence
  synthetic oligonucleotide
ctgttgtctgttccttccagctgtaaaaaaaaaactccgattggacactctt

SEQ ID NO: 317 - DNA, 52 bp, Artificial sequence
  synthetic oligonucleotide
aaggcttgctggataccactgtttaaaaaaaaaagacgctaatcaacgcgtt

SEQ ID NO: 318 - DNA, 52 bp, Artificial sequence
  synthetic oligonucleotide
aaagctgctggcaacctgagtttaaaaaaaaaaactccgattggacactctt

SEQ ID NO: 319 - DNA, 52 bp, Artificial sequence
  synthetic oligonucleotide
ggatttgcccaaccacattcgtttaaaaaaaaaagacgctaatcaacgcgtt

SEQ ID NO: 320 - DNA, 52 bp, Artificial sequence
  synthetic oligonucleotide
atatcatagcacacagaggggccaaaaaaaaaaactccgattggacactctt

SEQ ID NO: 321 - DNA, 52 bp, Artificial sequence
  synthetic oligonucleotide
tgagccccaaagtcaacgaagattaaaaaaaaaagacgctaatcaacctggt

SEQ ID NO: 322 - DNA, 52 bp, Artificial sequence
  synthetic oligonucleotide
gagtccgaagaagagagaggggtaaaaaaaaaaactcgatctggacactctt

SEQ ID NO: 323 - DNA, 52 bp, Artificial sequence
  synthetic oligonucleotide
taggtccaggaggtcgttgaactcaaaaaaaaaagacgctaatcaacctggt

SEQ ID NO: 324 - DNA, 52 bp, Artificial sequence
  synthetic oligonucleotide
ctggtgggttagcgagttggaaagaaaaaaaaaactcgatctggacactctt

SEQ ID NO: 325 - DNA, 52 bp, Artificial sequence
  synthetic oligonucleotide
ccgggcatgttcaagttggatttgaaaaaaaaaagacgctaatcaacctggt

SEQ ID NO: 326 - DNA, 52 bp, Artificial sequence
  synthetic oligonucleotide
ccagtcaccccttggcattttgtaaaaaaaaaaactcgatctggacactctt

SEQ ID NO: 327 - DNA, 52 bp, Artificial sequence
  synthetic oligonucleotide
actgaactctctctcctggcagtgaaaaaaaaaagacgctaatcaacctggt

SEQ ID NO: 328 - DNA, 52 bp, Artificial sequence
  synthetic oligonucleotide
cgtgggaagacagtgtgaaaggttaaaaaaaaaactcgatctggacactctt

SEQ ID NO: 329 - DNA, 52 bp, Artificial sequence
  synthetic oligonucleotide
ggtacacgatgcccaagatgagaaaaaaaaaaaagacgctaatctgtagacc

SEQ ID NO: 330 - DNA, 52 bp, Artificial sequence
  synthetic oligonucleotide
tatgccagcttgtcatcgtatgggaaaaaaaaaatctatccgggacactctt

SEQ ID NO: 331 - DNA, 52 bp, Artificial sequence
  synthetic oligonucleotide
gctgggtctctctgctatcctacaaaaaaaaaaagacgctaatctgtagacc

SEQ ID NO: 332 - DNA, 52 bp, Artificial sequence
  synthetic oligonucleotide
ttccgaggtgcaatgagactttccaaaaaaaaaatctatccgggacactctt

SEQ ID NO: 333 - DNA, 52 bp, Artificial sequence
  synthetic oligonucleotide
gaggacttgtcatgaaagtggcgtaaaaaaaaaagacgctaatctgtagacc

SEQ ID NO: 334 - DNA, 52 bp, Artificial sequence
  synthetic oligonucleotide
ggaagggatcagagcaggtaaagcaaaaaaaaaatctatccgggacactctt

SEQ ID NO: 335 - DNA, 52 bp, Artificial sequence
  synthetic oligonucleotide
gtgaccagtctcctagaggtgtcaaaaaaaaaaagacgctaatctgtagacc

SEQ ID NO: 336 - DNA, 52 bp, Artificial sequence
  synthetic oligonucleotide
ggctctctggggaagtttagcattaaaaaaaaaatctatccgggacactctt

SEQ ID NO: 337 - DNA, 52 bp, Artificial sequence
  synthetic oligonucleotide
acagcttctctttgatgtcacgcaaaaaaaaaaagacgctaatctacatggc

SEQ ID NO: 338 - DNA, 52 bp, Artificial sequence
  synthetic oligonucleotide
gccatctcctgctcgaagtctagaaaaaaaaaaaccgcttatggacactctt

SEQ ID NO: 339 - DNA, 52 bp, Artificial sequence
  synthetic oligonucleotide
acggatgtcaacgtcacacttcataaaaaaaaaagacgctaatctacatggc

SEQ ID NO: 340 - DNA, 52 bp, Artificial sequence
  synthetic oligonucleotide
agacagcactgtgttggcatagagaaaaaaaaaaccgcttatggacactctt

SEQ ID NO: 341 - DNA, 52 bp, Artificial sequence
  synthetic oligonucleotide
caatgcctgggtacatggtggtacaaaaaaaaaagacgctaatctacatggc

SEQ ID NO: 342 - DNA, 52 bp, Artificial sequence
  synthetic oligonucleotide
gccagagcagtaatctccttctgcaaaaaaaaaaccgcttatggacactctt

SEQ ID NO: 343 - DNA, 52 bp, Artificial sequence
  synthetic oligonucleotide
cctgagtcaaaagcgccaaaacaaaaaaaaaaaagacgctaatctacatggc

SEQ ID NO: 344 - DNA, 52 bp, Artificial sequence
  synthetic oligonucleotide
tcgccttcaccgttccagtttttaaaaaaaaaaaccgcttatggacactctt

SEQ ID NO: 345 - DNA, 52 bp, Artificial sequence
  synthetic oligonucleotide
gcgcctaacgtaccactagaacttaaaaaaaaaagacgctaatctcggaatc

SEQ ID NO: 346 - DNA, 52 bp, Artificial sequence
  synthetic oligonucleotide
taaagacttttgcgaactccctgcaaaaaaaaaatgactctcggacactctt

SEQ ID NO: 347 - DNA, 52 bp, Artificial sequence
  synthetic oligonucleotide
agtcccccaaaaagaagtcccaagaaaaaaaaaagacgctaatctcggaatc

SEQ ID NO: 348 - DNA, 51 bp, Artificial sequence
  synthetic oligonucleotide
ccgccctcaggttttctctgtacaaaaaaaaaatgactctcggacactctt

SEQ ID NO: 349 - DNA, 53 bp, Artificial sequence
  synthetic oligonucleotide
gcgttaatttggatgggattggtggaaaaaaaaaagacgctaatctcggaatc

SEQ ID NO: 350 - DNA, 52 bp, Artificial sequence
  synthetic oligonucleotide
agttttctagtcggcatcacggttaaaaaaaaaatgactctcggacactctt

SEQ ID NO: 351 - DNA, 52 bp, Artificial sequence
  synthetic oligonucleotide
aatctctccccttctccagttcgcaaaaaaaaaagacgctaatctcggaatc

SEQ ID NO: 352 - DNA, 52 bp, Artificial sequence
  synthetic oligonucleotide
acccctcccaattcccttgtatctaaaaaaaaaatgactctcggacactctt

SEQ ID NO: 353 - DNA, 52 bp, Artificial sequence
  synthetic oligonucleotide
agaactggtacacagcaaaccacaaaaaaaaaaagacgctaatccaggatct

SEQ ID NO: 354 - DNA, 52 bp, Artificial sequence
  synthetic oligonucleotide
gaatccacacttccctaggccctaaaaaaaaaaacttctgcaggacactctt

SEQ ID NO: 355 - DNA, 52 bp, Artificial sequence
  synthetic oligonucleotide
aaaggtcacacccagcaaagaacaaaaaaaaaaagacgctaatccaggatct

SEQ ID NO: 356 - DNA, 52 bp, Artificial sequence
  synthetic oligonucleotide
tggtttcagcatggaaccctgaagaaaaaaaaaacttctgcaggacactctt

SEQ ID NO: 357 - DNA, 52 bp, Artificial sequence
  synthetic oligonucleotide
ctgttgatctaaccagtggcagcaaaaaaaaaaagacgctaatccaggatct

SEQ ID NO: 358 - DNA, 52 bp, Artificial sequence
  synthetic oligonucleotide
atgcaggcaaggttcagtgtctagaaaaaaaaaacttctgcaggacactctt

SEQ ID NO: 359 - DNA, 52 bp, Artificial sequence
  synthetic oligonucleotide
tgacgtatggagattgagctgtgcaaaaaaaaaagacgctaatccaggatct

SEQ ID NO: 360 - DNA, 52 bp, Artificial sequence
  synthetic oligonucleotide
aagacacttttggaccgatctggcaaaaaaaaaacttctgcaggacactctt

SEQ ID NO: 361 - DNA, 52 bp, Artificial sequence
  synthetic oligonucleotide
cgtcttgctttagggtcagtctgtaaaaaaaaaagacgctaatcatcagcgt

SEQ ID NO: 362 - DNA, 52 bp, Artificial sequence
  synthetic oligonucleotide
actctggtattctggactggccttaaaaaaaaaaccattcgtggacactctt

SEQ ID NO: 363 - DNA, 52 bp, Artificial sequence
  synthetic oligonucleotide
gcttcgtcccctttgtcatgtactaaaaaaaaaagacgctaatcatcagcgt

SEQ ID NO: 364 - DNA, 52 bp, Artificial sequence
  synthetic oligonucleotide
tctcttgcgtgacctctctcttctaaaaaaaaaaccattcgtggacactctt

SEQ ID NO: 365 - DNA, 52 bp, Artificial sequence
  synthetic oligonucleotide
actctaggtatccgtcagggaagcaaaaaaaaaagacgctaatcatcagcgt

SEQ ID NO: 366 - DNA, 52 bp, Artificial sequence
  synthetic oligonucleotide
gggttcggaaaactcacctcgtataaaaaaaaaaccattcgtggacactctt

SEQ ID NO: 367 - DNA, 52 bp, Artificial sequence
  synthetic oligonucleotide
acactccagcatcgataagacaccaaaaaaaaaagacgctaatcatcagcgt

SEQ ID NO: 368 - DNA, 52 bp, Artificial sequence
  synthetic oligonucleotide
tatccctcagcttcttcttgcaccaaaaaaaaaaccattcgtggacactctt

SEQ ID NO: 369 - DNA, 52 bp, Artificial sequence
  synthetic oligonucleotide
ggagttattgttggccccatgagtaaaaaaaaaagacgctaatcgccatgat

SEQ ID NO: 370 - DNA, 52 bp, Artificial sequence
  synthetic oligonucleotide
gttctctatggccagcttccttccaaaaaaaaaatcgctactggacactctt

SEQ ID NO: 371 - DNA, 52 bp, Artificial sequence
  synthetic oligonucleotide
gctatggtcacaagccacatcactaaaaaaaaaagacgctaatcgccatgat

SEQ ID NO: 372 - DNA, 52 bp, Artificial sequence
  synthetic oligonucleotide
actctcgtgattgtaggattgcccaaaaaaaaaatcgctactggacactctt

SEQ ID NO: 373 - DNA, 52 bp, Artificial sequence
  synthetic oligonucleotide
cgcattagtcaccacagaaggacaaaaaaaaaaagacgctaatcgccatgat

SEQ ID NO: 374 - DNA, 52 bp, Artificial sequence
  synthetic oligonucleotide
ggaggcataaaaccaggtccctacaaaaaaaaaatcgctactggacactctt

SEQ ID NO: 375 - DNA, 52 bp, Artificial sequence
  synthetic oligonucleotide
aggaagcagcaggagaaattgtggaaaaaaaaaagacgctaatcgccatgat

SEQ ID NO: 376 - DNA, 52 bp, Artificial sequence
  synthetic oligonucleotide
ggaggcatactctaagggctctgaaaaaaaaaaatcgctactggacactctt

SEQ ID NO: 377 - DNA, 52 bp, Artificial sequence
  synthetic oligonucleotide
cacctcatccttgtgggtatagccaaaaaaaaaagacgctaatcctcgaatg

SEQ ID NO: 378 - DNA, 53 bp, Artificial sequence
  synthetic oligonucleotide
ggtgtccttgtaaaactcctggagtaaaaaaaaaatctcacgtggacactctt

SEQ ID NO: 379 - DNA, 52 bp, Artificial sequence
  synthetic oligonucleotide
gggttcatccttgctccgtaacttaaaaaaaaaagacgctaatcctcgaatg

SEQ ID NO: 380 - DNA, 52 bp, Artificial sequence
  synthetic oligonucleotide
atggatggctttgagtgtttcccgaaaaaaaaaatctcacgtggacactctt

SEQ ID NO: 381 - DNA, 53 bp, Artificial sequence
  synthetic oligonucleotide
ggtgtccgagataaactgctccaaaaaaaaaaaaagacgctaatcctcgaatg

SEQ ID NO: 382 - DNA, 52 bp, Artificial sequence
  synthetic oligonucleotide
tttccaaaagctgtttcttggggcaaaaaaaaaatctcacgtggacactctt

SEQ ID NO: 383 - DNA, 52 bp, Artificial sequence
  synthetic oligonucleotide
ggttgggcagactctagaccatttaaaaaaaaaagacgctaatcctcgaatg

SEQ ID NO: 384 - DNA, 50 bp, Artificial sequence
  synthetic oligonucleotide
gtcttcagggccgttgttcctgaaaaaaaaaatctcacgtggacactctt

SEQ ID NO: 385 - DNA, 52 bp, Artificial sequence
  synthetic oligonucleotide
tggttccaccttctccaacttcacaaaaaaaaaagacgctaatctcaatcgg

SEQ ID NO: 386 - DNA, 51 bp, Artificial sequence
  synthetic oligonucleotide
gctttcatgtcctgggactcctcaaaaaaaaaattctccagggacactctt

SEQ ID NO: 387 - DNA, 52 bp, Artificial sequence
  synthetic oligonucleotide
aggttctcattgttgtcggcttccaaaaaaaaaagacgctaatctcaatcgg

SEQ ID NO: 388 - DNA, 53 bp, Artificial sequence
  synthetic oligonucleotide
gggtctccgatttgcatatctcctgaaaaaaaaaattctccagggacactctt

SEQ ID NO: 389 - DNA, 52 bp, Artificial sequence
  synthetic oligonucleotide
gattggcgatgtgagtgatctgctaaaaaaaaaagacgctaatctcaatcgg

SEQ ID NO: 390 - DNA, 52 bp, Artificial sequence
  synthetic oligonucleotide
aaccacatccttctctagcccaagaaaaaaaaaattctccagggacactctt

SEQ ID NO: 391 - DNA, 52 bp, Artificial sequence
  synthetic oligonucleotide
acggttctcaatgctagttcgcttaaaaaaaaaagacgctaatctcaatcgg

SEQ ID NO: 392 - DNA, 52 bp, Artificial sequence
  synthetic oligonucleotide
gaaacatggtctccagactccaccaaaaaaaaaattctccagggacactctt

SEQ ID NO: 393 - DNA, 52 bp, Artificial sequence
  synthetic oligonucleotide
gaagctcacaaaagtagtcgccctaaaaaaaaaagacgctaatatcgtgacc

SEQ ID NO: 394 - DNA, 52 bp, Artificial sequence
  synthetic oligonucleotide
ttattggagctcatgggattcgcgaaaaaaaaaactcagtcgtgacactctt

SEQ ID NO: 395 - DNA, 52 bp, Artificial sequence
  synthetic oligonucleotide
tctttcaggcatctggcttggttgaaaaaaaaaagacgctaatatcgtgacc

SEQ ID NO: 396 - DNA, 52 bp, Artificial sequence
  synthetic oligonucleotide
cacagtccaacttccctcatccataaaaaaaaaactcagtcgtgacactctt

SEQ ID NO: 397 - DNA, 52 bp, Artificial sequence
  synthetic oligonucleotide
cccctccccgatgattctttcaacaaaaaaaaaagacgctaatatcgtgacc

SEQ ID NO: 398 - DNA, 52 bp, Artificial sequence
  synthetic oligonucleotide
tcagcagagctctcccatcttgagaaaaaaaaaactcagtcgtgacactctt

SEQ ID NO: 399 - DNA, 52 bp, Artificial sequence
  synthetic oligonucleotide
agatcctggagtcagagttctggcaaaaaaaaaagacgctaatatcgtgacc

SEQ ID NO: 400 - DNA, 52 bp, Artificial sequence
  synthetic oligonucleotide
atggttctaggatccccttcctgcaaaaaaaaaactcagtcgtgacactctt

SEQ ID NO: 401 - DNA, 52 bp, Artificial sequence
  synthetic oligonucleotide
cctcagaactaggcaaactgtgggaaaaaaaaaagacgctaatcaggctact

SEQ ID NO: 402 - DNA, 52 bp, Artificial sequence
  synthetic oligonucleotide
gatgaggcgttcccagaattcgataaaaaaaaaactaccttgggacactctt

SEQ ID NO: 403 - DNA, 52 bp, Artificial sequence
  synthetic oligonucleotide
aggctgaggtacttctgcttctgaaaaaaaaaaagacgctaatcaggctact

SEQ ID NO: 404 - DNA, 52 bp, Artificial sequence
  synthetic oligonucleotide
atggaggagagttcttgcatctgcaaaaaaaaaactaccttgggacactctt

SEQ ID NO: 405 - DNA, 52 bp, Artificial sequence
  synthetic oligonucleotide
gcagagaagttttgctgcaactgtaaaaaaaaaagacgctaatcaggctact

SEQ ID NO: 406 - DNA, 52 bp, Artificial sequence
  synthetic oligonucleotide
agtggcttccaaattcacctccaaaaaaaaaaaactaccttgggacactctt

SEQ ID NO: 407 - DNA, 52 bp, Artificial sequence
  synthetic oligonucleotide
aagcccagatgttgcgtaagtctcaaaaaaaaaagacgctaatcaggctact

SEQ ID NO: 408 - DNA, 52 bp, Artificial sequence
  synthetic oligonucleotide
ggaagaaggaaggaacctggctttaaaaaaaaaactaccttgggacactctt

SEQ ID NO: 409 - DNA, 49 bp, Artificial sequence; identical to SEQ ID NO: 111
  synthetic oligonucleotide

SEQ ID NO: 410 - DNA, 48 bp, Artificial sequence; identical to SEQ ID NO: 112
  synthetic oligonucleotide

SEQ ID NO: 411 - DNA, 50 bp, Artificial sequence; identical to SEQ ID NO: 113
  synthetic oligonucleotide

SEQ ID NO: 412 - DNA, 49 bp, Artificial sequence; identical to SEQ ID NO: 114
  synthetic oligonucleotide

SEQ ID NO: 413 - DNA, 47 bp, Artificial sequence; identical to SEQ ID NO: 115
  synthetic oligonucleotide

SEQ ID NO: 414 - DNA, 48 bp, Artificial sequence; identical to SEQ ID NO: 116
  synthetic oligonucleotide

SEQ ID NO: 415 - DNA, 49 bp, Artificial sequence; identical to SEQ ID NO: 117
  synthetic oligonucleotide

SEQ ID NO: 416 - DNA, 48 bp, Artificial sequence; identical to SEQ ID NO: 118
  synthetic oligonucleotide

SEQ ID NO: 417 - DNA, 47 bp, Artificial sequence
  synthetic oligonucleotide
atgtgatgtcacccgaagcaaaaaaaaaagacgctaatctgtagacc

SEQ ID NO: 418 - DNA, 47 bp, Artificial sequence
  synthetic oligonucleotide
ggagcctgatttcgcatttaaaaaaaaaatctatccgggacactctt

SEQ ID NO: 419 - DNA, 47 bp, Artificial sequence
  synthetic oligonucleotide
caacctcttgcccgagaacaaaaaaaaaagacgctaatctgtagacc

SEQ ID NO: 420 - DNA, 47 bp, Artificial sequence
  synthetic oligonucleotide
agggtgaggacgaaggtgtaaaaaaaaaatctatccgggacactctt

SEQ ID NO: 421 - DNA, 48 bp, Artificial sequence
  synthetic oligonucleotide
ttgtctcccgatttgaccacaaaaaaaaaagacgctaatctgtagacc

SEQ ID NO: 422 - DNA, 48 bp, Artificial sequence
  synthetic oligonucleotide
agacgtatctcgccgaaaggaaaaaaaaaatctatccgggacactctt

SEQ ID NO: 423 - DNA, 48 bp, Artificial sequence
  synthetic oligonucleotide
gaactctgcgggtagctctgaaaaaaaaaagacgctaatctgtagacc

SEQ ID NO: 424 - DNA, 49 bp, Artificial sequence
  synthetic oligonucleotide
tccagctctctcagcatgattaaaaaaaaaatctatccgggacactctt

SEQ ID NO: 425 - DNA, 48 bp, Artificial sequence; identical to SEQ ID NO: 135
  synthetic oligonucleotide

SEQ ID NO: 426 - DNA, 48 bp, Artificial sequence; identical to SEQ ID NO: 136
  synthetic oligonucleotide

SEQ ID NO: 427 - DNA, 48 bp, Artificial sequence; identical to SEQ ID NO: 137
  synthetic oligonucleotide

SEQ ID NO: 428 - DNA, 48 bp, Artificial sequence; identical to SEQ ID NO: 138
  synthetic oligonucleotide

SEQ ID NO: 429 - DNA, 48 bp, Artificial sequence; identical to SEQ ID NO: 139
  synthetic oligonucleotide

SEQ ID NO: 430 - DNA, 48 bp, Artificial sequence; identical to SEQ ID NO: 140
  synthetic oligonucleotide

SEQ ID NO: 431 - DNA, 47 bp, Artificial sequence; identical to SEQ ID NO: 141
  synthetic oligonucleotide

SEQ ID NO: 432 - DNA, 48 bp, Artificial sequence; identical to SEQ ID NO: 142
  synthetic oligonucleotide

SEQ ID NO: 433 - DNA, 48 bp, Artificial sequence
  synthetic oligonucleotide
tgacaatgagaattttggcgaaaaaaaaaagacgctaatcatcagcgt

SEQ ID NO: 434 - DNA, 48 bp, Artificial sequence
  synthetic oligonucleotide
gccttctttcttcccatcagaaaaaaaaaaccattcgtggacactctt

SEQ ID NO: 435 - DNA, 48 bp, Artificial sequence
  synthetic oligonucleotide
aaacaggtagacagcaccccaaaaaaaaaagacgctaatcatcagcgt

SEQ ID NO: 436 - DNA, 47 bp, Artificial sequence
  synthetic oligonucleotide
atgctgggtcccaagactcaaaaaaaaaaccattcgtggacactctt

SEQ ID NO: 437 - DNA, 47 bp, Artificial sequence
  synthetic oligonucleotide
actcggactcggctcagacaaaaaaaaaagacgctaatcatcagcgt

SEQ ID NO: 438 - DNA, 48 bp, Artificial sequence
  synthetic oligonucleotide
tttcacagtgtgccttcagcaaaaaaaaaaccattcgtggacactctt

SEQ ID NO: 439 - DNA, 48 bp, Artificial sequence
  synthetic oligonucleotide
gatatggtcggctccacagtaaaaaaaaaagacgctaatcatcagcgt

SEQ ID NO: 440 - DNA, 48 bp, Artificial sequence
  synthetic oligonucleotide
gagatgccgagattgtcctgaaaaaaaaaaccattcgtggacactctt

SEQ ID NO: 441 - DNA, 52 bp, Artificial sequence; identical to SEQ ID NO: 97
  synthetic oligonucleotide

SEQ ID NO: 442 - DNA, 52 bp, Artificial sequence; identical to SEQ ID NO: 98
  synthetic oligonucleotide

SEQ ID NO: 443 - DNA, 52 bp, Artificial sequence; identical to SEQ ID NO: 99
  synthetic oligonucleotide

SEQ ID NO: 444 - DNA, 52 bp, Artificial sequence; identical to SEQ ID NO: 100
  synthetic oligonucleotide

SEQ ID NO: 445 - DNA, 53 bp, Artificial sequence; identical to SEQ ID NO: 101
  synthetic oligonucleotide

SEQ ID NO: 446 - DNA, 52 bp, Artificial sequence; identical to SEQ ID NO: 102
  synthetic oligonucleotide

SEQ ID NO: 447 - DNA, 52 bp, Artificial sequence; identical to SEQ ID NO: 215
  synthetic oligonucleotide

SEQ ID NO: 448 - DNA, 52 bp, Artificial sequence; identical to SEQ ID NO: 216
  synthetic oligonucleotide

SEQ ID NO: 449 - DNA, 52 bp, Artificial sequence; identical to SEQ ID NO: 217
  synthetic oligonucleotide

SEQ ID NO: 450 - DNA, 52 bp, Artificial sequence; identical to SEQ ID NO: 218
  synthetic oligonucleotide

SEQ ID NO: 451 - DNA, 52 bp, Artificial sequence; identical to SEQ ID NO: 219
  synthetic oligonucleotide

SEQ ID NO: 452 - DNA, 52 bp, Artificial sequence; identical to SEQ ID NO: 220
  synthetic oligonucleotide

SEQ ID NO: 453 - DNA, 52 bp, Artificial sequence; identical to SEQ ID NO: 283
  synthetic oligonucleotide

SEQ ID NO: 454 - DNA, 52 bp, Artificial sequence; identical to SEQ ID NO: 284
  synthetic oligonucleotide

SEQ ID NO: 455 - DNA, 52 bp, Artificial sequence; identical to SEQ ID NO: 285
  synthetic oligonucleotide

SEQ ID NO: 456 - DNA, 52 bp, Artificial sequence; identical to SEQ ID NO: 286
  synthetic oligonucleotide

SEQ ID NO: 457 - DNA, 52 bp, Artificial sequence; identical to SEQ ID NO: 287
  synthetic oligonucleotide

SEQ ID NO: 458 - DNA, 52 bp, Artificial sequence; identical to SEQ ID NO: 288
  synthetic oligonucleotide

SEQ ID NO: 459 - DNA, 52 bp, Artificial sequence
  synthetic oligonucleotide
cctggaggggagatagagcttctcaaaaaaaaaagacgctaatatcgtgacc

SEQ ID NO: 460 - DNA, 52 bp, Artificial sequence
  synthetic oligonucleotide
gcgcttgtggagaaggagttcataaaaaaaaaaactcagtcgtgacactctt

SEQ ID NO: 461 - DNA, 52 bp, Artificial sequence
  synthetic oligonucleotide
ttcaccaggcaagtctcctcattgaaaaaaaaaagacgctaatatcgtgacc

SEQ ID NO: 462 - DNA, 53 bp, Artificial sequence
  synthetic oligonucleotide
acctcaaactccaaaagaccagtgaaaaaaaaaaactcagtcgtgacactctt

SEQ ID NO: 463 - DNA, 53 bp, Artificial sequence
  synthetic oligonucleotide
tctggcttgttcctcactactctcaaaaaaaaaaagacgctaatatcgtgacc

SEQ ID NO: 464 - DNA, 53 bp, Artificial sequence
  synthetic oligonucleotide
ggacttttgtactcatctgcacagcaaaaaaaaaactcagtcgtgacactctt

SEQ ID NO: 465 - DNA, 52 bp, Artificial sequence; identical to SEQ ID NO: 97
  synthetic oligonucleotide

SEQ ID NO: 466 - DNA, 52 bp, Artificial sequence; identical to SEQ ID NO: 98
  synthetic oligonucleotide

SEQ ID NO: 467 - DNA, 52 bp, Artificial sequence; identical to SEQ ID NO: 99
  synthetic oligonucleotide

SEQ ID NO: 468 - DNA, 52 bp, Artificial sequence; identical to SEQ ID NO: 100
  synthetic oligonucleotide

SEQ ID NO: 469 - DNA, 53 bp, Artificial sequence; identical to SEQ ID NO: 101
  synthetic oligonucleotide

SEQ ID NO: 470 - DNA, 52 bp, Artificial sequence; identical to SEQ ID NO: 102
  synthetic oligonucleotide

SEQ ID NO: 471 - DNA, 52 bp, Artificial sequence; identical to SEQ ID NO: 215
  synthetic oligonucleotide

SEQ ID NO: 472 - DNA, 52 bp, Artificial sequence; identical to SEQ ID NO: 216
  synthetic oligonucleotide

SEQ ID NO: 473 - DNA, 52 bp, Artificial sequence; identical to SEQ ID NO: 217
  synthetic oligonucleotide

SEQ ID NO: 474 - DNA, 52 bp, Artificial sequence; identical to SEQ ID NO: 218
  synthetic oligonucleotide

SEQ ID NO: 475 - DNA, 52 bp, Artificial sequence; identical to SEQ ID NO: 219
  synthetic oligonucleotide

SEQ ID NO: 476 - DNA, 52 bp, Artificial sequence; identical to SEQ ID NO: 220
  synthetic oligonucleotide

SEQ ID NO: 477 - DNA, 52 bp, Artificial sequence
  synthetic oligonucleotide
gtgcacataagcctcgttatcccaaaaaaaaaaagacgctaatctcggaatc

SEQ ID NO: 478 - DNA, 52 bp, Artificial sequence
  synthetic oligonucleotide
gtgcagttcagtgatcgtacaggtaaaaaaaaaatgactctcggacactctt

SEQ ID NO: 479 - DNA, 52 bp, Artificial sequence
  synthetic oligonucleotide
caacacgcaggacaggtacagattaaaaaaaaaagacgctaatctcggaatc

SEQ ID NO: 480 - DNA, 52 bp, Artificial sequence
  synthetic oligonucleotide
tccagctgtagagtgggcttatcaaaaaaaaaaatgactctcggacactctt

SEQ ID NO: 481 - DNA, 52 bp, Artificial sequence
  synthetic oligonucleotide
gaagacgggcatgttttctgcttgaaaaaaaaaagacgctaatctcggaatc

SEQ ID NO: 482 - DNA, 52 bp, Artificial sequence
  synthetic oligonucleotide
gtcagttatatcctggccgcctttaaaaaaaaaatgactctcggacactctt

SEQ ID NO: 483 - DNA, 52 bp, Artificial sequence; identical to SEQ ID NO: 269
  synthetic oligonucleotide

SEQ ID NO: 484 - DNA, 52 bp, Artificial sequence; identical to SEQ ID NO: 270
  synthetic oligonucleotide

SEQ ID NO: 485 - DNA, 52 bp, Artificial sequence; identical to SEQ ID NO: 271
  synthetic oligonucleotide

SEQ ID NO: 486 - DNA, 53 bp, Artificial sequence; identical to SEQ ID NO: 272
  synthetic oligonucleotide

SEQ ID NO: 487 - DNA, 52 bp, Artificial sequence; identical to SEQ ID NO: 273
  synthetic oligonucleotide

SEQ ID NO: 488 - DNA, 51 bp, Artificial sequence; identical to SEQ ID NO: 274
  synthetic oligonucleotide

SEQ ID NO: 489 - DNA, 52 bp, Artificial sequence
  synthetic oligonucleotide
acgccaatgaaatgactccctctcaaaaaaaaaagacgctaatctgtagacc

SEQ ID NO: 490 - DNA, 52 bp, Artificial sequence
  synthetic oligonucleotide
tggccatcttgacttctttgctgaaaaaaaaaaatctatccgggacactctt

SEQ ID NO: 491 - DNA, 52 bp, Artificial sequence
  synthetic oligonucleotide
agaggaggttggtctcactacctgaaaaaaaaaagacgctaatctgtagacc

SEQ ID NO: 492 - DNA, 52 bp, Artificial sequence
  synthetic oligonucleotide
gttcttagtgccgtgagtttcccaaaaaaaaaaatctatccgggacactctt

SEQ ID NO: 493 - DNA, 52 bp, Artificial sequence
  synthetic oligonucleotide
aagcacaacttggaccaaaatgccaaaaaaaaaagacgctaatctgtagacc

SEQ ID NO: 494 - DNA, 52 bp, Artificial sequence
  synthetic oligonucleotide
aatgcagagtttcctggctatgggaaaaaaaaaatctatccgggacactctt

SEQ ID NO: 495 - DNA, 52 bp, Artificial sequence
  synthetic oligonucleotide
cctggaggggagatagagcttctcaaaaaaaaaagacgctaatcacacttgg

SEQ ID NO: 496 - DNA, 52 bp, Artificial sequence
  synthetic oligonucleotide
gcgcttgtggagaaggagttcataaaaaaaaaaatacgctctggacactctt

SEQ ID NO: 497 - DNA, 52 bp, Artificial sequence
  synthetic oligonucleotide
ttcaccaggcaagtctcctcattgaaaaaaaaaagacgctaatcacacttgg

SEQ ID NO: 498 - DNA, 53 bp, Artificial sequence
  synthetic oligonucleotide
acctcaaactccaaaagaccagtgaaaaaaaaaaatacgctctggacactctt

SEQ ID NO: 499 - DNA, 53 bp, Artificial sequence
  synthetic oligonucleotide
tctggcttgttcctcactactctcaaaaaaaaaaagacgctaatcacacttgg

SEQ ID NO: 500 - DNA, 53 bp, Artificial sequence
  synthetic oligonucleotide
ggacttttgtactcatctgcacagcaaaaaaaaaatacgctctggacactctt

SEQ ID NO: 501 - DNA, 52 bp, Artificial sequence
  synthetic oligonucleotide
caggttgttgtgacgccttctgagaaaaaaaaaagacgctaatcaggctact

SEQ ID NO: 502 - DNA, 52 bp, Artificial sequence
  synthetic oligonucleotide
gtcagttgaagaggaggcagagtcaaaaaaaaaactaccttgggacactctt

SEQ ID NO: 503 - DNA, 52 bp, Artificial sequence
  synthetic oligonucleotide
ggtaaagtactgcaggcagctgtaaaaaaaaaaagacgctaatcaggctact

SEQ ID NO: 504 - DNA, 52 bp, Artificial sequence
  synthetic oligonucleotide
gagccttgggagctgagaaacttcaaaaaaaaaactaccttgggacactctt

SEQ ID NO: 505 - DNA, 52 bp, Artificial sequence
  synthetic oligonucleotide
ggaaggtggaatgagggaggaagaaaaaaaaaaagacgctaatcaggctact

SEQ ID NO: 506 - DNA, 52 bp, Artificial sequence
  synthetic oligonucleotide
gtcatcaagtggcctgatggatccaaaaaaaaaactaccttgggacactctt

SEQ ID NO: 507 - DNA, 52 bp, Artificial sequence
  synthetic oligonucleotide
tcacacagagctgcagaaatcaggaaaaaaaaaagacgctaatctcaatcgg

SEQ ID NO: 508 - DNA, 52 bp, Artificial sequence
  synthetic oligonucleotide
tttagcactccttggcaaaactgcaaaaaaaaaattctccagggacactctt

SEQ ID NO: 509 - DNA, 52 bp, Artificial sequence
  synthetic oligonucleotide
ttgtggatcctggctagcagactaaaaaaaaaaagacgctaatctcaatcgg

SEQ ID NO: 510 - DNA, 52 bp, Artificial sequence
  synthetic oligonucleotide
agaaaccaaggcacagtggaacaaaaaaaaaaaattctccagggacactctt

SEQ ID NO: 511 - DNA, 52 bp, Artificial sequence
  synthetic oligonucleotide
tccagacagagctctcttccatcaaaaaaaaaaagacgctaatctcaatcgg

SEQ ID NO: 512 - DNA, 52 bp, Artificial sequence
  synthetic oligonucleotide
aaacttctccacaaccctctgcacaaaaaaaaaattctccagggacactctt

SEQ ID NO: 513 - DNA, 48 bp, Artificial sequence; identical to SEQ ID NO: 127
  synthetic oligonucleotide

SEQ ID NO: 514 - DNA, 47 bp, Artificial sequence; identical to SEQ ID NO: 128
  synthetic oligonucleotide

SEQ ID NO: 515 - DNA, 49 bp, Artificial sequence; identical to SEQ ID NO: 129
  synthetic oligonucleotide

SEQ ID NO: 516 - DNA, 48 bp, Artificial sequence; identical to SEQ ID NO: 130
  synthetic oligonucleotide

SEQ ID NO: 517 - DNA, 48 bp, Artificial sequence; identical to SEQ ID NO: 131
  synthetic oligonucleotide

SEQ ID NO: 518 - DNA, 48 bp, Artificial sequence; identical to SEQ ID NO: 132
  synthetic oligonucleotide

SEQ ID NO: 519 - DNA, 48 bp, Artificial sequence; identical to SEQ ID NO: 133
  synthetic oligonucleotide

SEQ ID NO: 520 - DNA, 49 bp, Artificial sequence; identical to SEQ ID NO: 134
  synthetic oligonucleotide

SEQ ID NO: 521 - DNA, 48 bp, Artificial sequence
  synthetic oligonucleotide
agagctacgagctgcctgacaaaaaaaaaagacgctaatcaacctggt

SEQ ID NO: 522 - DNA, 47 bp, Artificial sequence
  synthetic oligonucleotide
caggtcatcaccattggcaaaaaaaaaaactcgatctggacactctt

SEQ ID NO: 523 - DNA, 49 bp, Artificial sequence
  synthetic oligonucleotide
catgaagtgtgacgtggacataaaaaaaaaagacgctaatcaacctggt

SEQ ID NO: 524 - DNA, 48 bp, Artificial sequence
  synthetic oligonucleotide
caaagacctgtacgccaacaaaaaaaaaaactcgatctggacactctt

SEQ ID NO: 525 - DNA, 48 bp, Artificial sequence
  synthetic oligonucleotide
ctgtccaccttccagcagataaaaaaaaaagacgctaatcaacctggt

SEQ ID NO: 526 - DNA, 48 bp, Artificial sequence
  synthetic oligonucleotide
cagcaagcaggagtatgacgaaaaaaaaaactcgatctggacactctt

SEQ ID NO: 527 - DNA, 48 bp, Artificial sequence
  synthetic oligonucleotide
gcgttacaccctttcttgaaaaaaaaaaaagacgctaatcaacctggt

SEQ ID NO: 528 - DNA, 49 bp, Artificial sequence
  synthetic oligonucleotide
acctaacttgcgcagaaaacaaaaaaaaaaactcgatctggacactctt

SEQ ID NO: 529 - DNA, 48 bp, Artificial sequence
  synthetic oligonucleotide
cacacgcagctcattgtagaaaaaaaaaaagacgctaatcaacctggt

SEQ ID NO: 530 - DNA, 47 bp, Artificial sequence
  synthetic oligonucleotide
tgccaatggtgatgacctgaaaaaaaaaagacgctaatcaacctggt

SEQ ID NO: 531 - DNA, 48 bp, Artificial sequence
  synthetic oligonucleotide
tgttggcgtacaggtctttgaaaaaaaaaagacgctaatcaacctggt

SEQ ID NO: 532 - DNA, 48 bp, Artificial sequence
  synthetic oligonucleotide
tgatctccttctgcatcctgaaaaaaaaaagacgctaatcaacctggt

SEQ ID NO: 533 - DNA, 48 bp, Artificial sequence
  synthetic oligonucleotide
cgtcatactcctgcttgctgaaaaaaaaaagacgctaatcaacctggt

SEQ ID NO: 534 - DNA, 49 bp, Artificial sequence
  synthetic oligonucleotide
tgttttctgcgcaagttaggtaaaaaaaaaagacgctaatcaacctggt

SEQ ID NO: 535 - DNA, 47 bp, Artificial sequence
  synthetic oligonucleotide
gtgaactttgggggatgctaaaaaaaaaagacgctaatcaacctggt

SEQ ID NO: 536 - DNA, 48 bp, Artificial sequence
  synthetic oligonucleotide
cttgaggcctgagctacgtgaaaaaaaaaactcgatctggacactctt

SEQ ID NO: 537 - DNA, 48 bp, Artificial sequence
  synthetic oligonucleotide
caaaagaagatgcggctgacaaaaaaaaaactcgatctggacactctt

SEQ ID NO: 538 - DNA, 50 bp, Artificial sequence
  synthetic oligonucleotide
tgatggcaacaatatccactttaaaaaaaaaactcgatctggacactctt

SEQ ID NO: 539 - DNA, 48 bp, Artificial sequence
  synthetic oligonucleotide
tggaagatggtgatgggattaaaaaaaaaactcgatctggacactctt

SEQ ID NO: 540 - DNA, 51 bp, Artificial sequence
  synthetic oligonucleotide
tctgtgtccagaaaggcaaagccaaaaaaaaaagacgctaatatcgtgacc

SEQ ID NO: 541 - DNA, 47 bp, Artificial sequence
  synthetic oligonucleotide
gtgggggaaggtgttgggcaaaaaaaaaactcagtcgtgacactctt

SEQ ID NO: 542 - DNA, 50 bp, Artificial sequence
  synthetic oligonucleotide
tcagaggcattaaggtaggcataaaaaaaaaagacgctaatatcgtgacc

SEQ ID NO: 543 - DNA, 50 bp, Artificial sequence
  synthetic oligonucleotide
gcttccagaagggctcagagtgaaaaaaaaaactcagtcgtgacactctt

SEQ ID NO: 544 - DNA, 53 bp, Artificial sequence
  synthetic oligonucleotide
caggagcagtacatgaattatgagaaaaaaaaaaagacgctaatatcgtgacc

SEQ ID NO: 545 - DNA, 53 bp, Artificial sequence
  synthetic oligonucleotide
tcaacccctggtggcacatctaataaaaaaaaaaactcagtcgtgacactctt

SEQ ID NO: 546 - DNA, 48 bp, Artificial sequence
  synthetic oligonucleotide
ttgccaatggtgatgacctgaaaaaaaaaagacgctaatatcgtgacc

SEQ ID NO: 547 - DNA, 48 bp, Artificial sequence
  synthetic oligonucleotide
gcctcagggcagcggaaccgaaaaaaaaaactcagtcgtgacactctt

SEQ ID NO: 548 - DNA, 46 bp, Artificial sequence
  synthetic oligonucleotide
gggcgacgtagcacagctaaaaaaaaaagacgctaatatcgtgacc

SEQ ID NO: 549 - DNA, 52 bp, Artificial sequence
  synthetic oligonucleotide
ccgtggccatctcttgctcgaagtaaaaaaaaaactcagtcgtgacactctt

SEQ ID NO: 550 - DNA, 46 bp, Artificial sequence
  synthetic oligonucleotide
ggcctcggtcagcagcacaaaaaaaaaagacgctaatatcgtgacc

SEQ ID NO: 551 - DNA, 49 bp, Artificial sequence
  synthetic oligonucleotide
cgcggttggccttggggttcaaaaaaaaaaactcagtcgtgacactctt

SEQ ID NO: 552 - DNA, 52 bp, Artificial sequence
  synthetic oligonucleotide
tgtcacagctatgatggtgaggagaaaaaaaaaagacgctaatatcgtgacc

SEQ ID NO: 553 - DNA, 53 bp, Artificial sequence
  synthetic oligonucleotide
catcgtaggttgcatagagtgcgataaaaaaaaaactcagtcgtgacactctt

SEQ ID NO: 554 - DNA, 50 bp, Artificial sequence
  synthetic oligonucleotide
caaccagcctccgcaagcatataaaaaaaaaagacgctaatatcgtgacc

SEQ ID NO: 555 - DNA, 52 bp, Artificial sequence
  synthetic oligonucleotide
ctggtctcgggaatgacattacgtaaaaaaaaaactcagtcgtgacactctt

SEQ ID NO: 556 - DNA, 48 bp, Artificial sequence
  synthetic oligonucleotide
tggatcagtcgagcagctgaaaaaaaaaaagacgctaatatcgtgacc

SEQ ID NO: 557 - DNA, 50 bp, Artificial sequence
  synthetic oligonucleotide
gtacaaggctcagtggtggcataaaaaaaaaactcagtcgtgacactctt

SEQ ID NO: 558 - DNA, 50 bp, Artificial sequence
  synthetic oligonucleotide
agaatgccggctgggaagactaaaaaaaaaaagacgctaatatcgtgacc

SEQ ID NO: 559 - DNA, 51 bp, Artificial sequence
  synthetic oligonucleotide
ggactgctgggcactaaagaaggaaaaaaaaaactcagtcgtgacactctt

SEQ ID NO: 560 - DNA, 53 bp, Artificial sequence
  synthetic oligonucleotide
cgtgtcctatgaccatgccgatgccaaaaaaaaaagacgctaatatcgtgacc

SEQ ID NO: 561 - DNA, 53 bp, Artificial sequence
  synthetic oligonucleotide
gccattgtcatcgaagccatgggtgaaaaaaaaaactcagtcgtgacactctt

SEQ ID NO: 562 - DNA, 52 bp, Artificial sequence
  synthetic oligonucleotide
ccacacctgtgcaaagttcaagaaaaaaaaaaaagacgctaatatcgtgacc

SEQ ID NO: 563 - DNA, 53 bp, Artificial sequence
  synthetic oligonucleotide
cgcatactctggcctataggttccaaaaaaaaaaactcagtcgtgacactctt

SEQ ID NO: 564 - DNA, 47 bp, Artificial sequence
  synthetic oligonucleotide
aggcccagaactctccagtaaaaaaaaaagacgctaatatcgtgacc

SEQ ID NO: 565 - DNA, 51 bp, Artificial sequence
  synthetic oligonucleotide
ccaaacgccaactgataagaggcaaaaaaaaaactcagtcgtgacactctt

SEQ ID NO: 566 - DNA, 52 bp, Artificial sequence
  synthetic oligonucleotide
actgtggttccagagatggagactaaaaaaaaaagacgctaatatcgtgacc

SEQ ID NO: 567 - DNA, 53 bp, Artificial sequence
  synthetic oligonucleotide
caggatactgagggcatgtcaatataaaaaaaaaactcagtcgtgacactctt

SEQ ID NO: 568 - DNA, 53 bp, Artificial sequence
  synthetic oligonucleotide
tccatctccatgcagttctcacacaaaaaaaaaaagacgctaatatcgtgacc

SEQ ID NO: 569 - DNA, 53 bp, Artificial sequence
  synthetic oligonucleotide
tgacaattgtggccaccgacatcacaaaaaaaaaactcagtcgtgacactctt

SEQ ID NO: 570 - DNA, 50 bp, Artificial sequence
  synthetic oligonucleotide
cccagtgatgcagatgtccactaaaaaaaaaagacgctaatatcgtgacc

SEQ ID NO: 571 - DNA, 51 bp, Artificial sequence
  synthetic oligonucleotide
cagtagtaaaccagcagcagcaaaaaaaaaaaactcagtcgtgacactctt

SEQ ID NO: 572 - DNA, 48 bp, Artificial sequence
  synthetic oligonucleotide
gaacaggtggtggcctctccaaaaaaaaaagacgctaatatcgtgacc

SEQ ID NO: 573 - DNA, 53 bp, Artificial sequence
  synthetic oligonucleotide
tggcctttccggatgggctcatagtaaaaaaaaaactcagtcgtgacactctt

SEQ ID NO: 574 - DNA, 52 bp, Artificial sequence
  synthetic oligonucleotide
ccacagggctgtttgtgagcacagaaaaaaaaaagacgctaatcagatgcct

SEQ ID NO: 575 - DNA, 51 bp, Artificial sequence
  synthetic oligonucleotide
atgaggacgttgggctctctcagaaaaaaaaaacacttgtcggacactctt

SEQ ID NO: 576 - DNA, 49 bp, Artificial sequence
  synthetic oligonucleotide
tgggcaggaagactgtctctgaaaaaaaaaagacgctaatcagatgcct

SEQ ID NO: 577 - DNA, 52 bp, Artificial sequence
  synthetic oligonucleotide
tggaacttgcggaaaaggtggtctaaaaaaaaaacacttgtcggacactctt

SEQ ID NO: 578 - DNA, 47 bp, Artificial sequence
  synthetic oligonucleotide
ctcagttgagggcaggaagaaaaaaaaaagacgctaatcagatgcct

SEQ ID NO: 579 - DNA, 53 bp, Artificial sequence
  synthetic oligonucleotide
agtgctccaccctgcagtcgtaaacaaaaaaaaaacacttgtcggacactctt